(19) United States
(12) Patent Application Publication
(10) Pub. No.: US 2005/0089933 A1
(43) Pub. Date: Apr. 28, 2005

(54) Compositions and methods for surrogate antibody modulation of an immune response and transport

(75) Inventors: Stephen B. Friedman, Chapel Hill, NC (US); David J. Drutz, Chapel Hill, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Syntherica Corporation, Durham, NC (US)

(21) Appl. No.: 10/912,448

(22) Filed: Aug. 5, 2004

Related U.S. Application Data
(63) Continuation of application No. PCT/US03/05000, filed on Feb. 19, 2003.
(60) Provisional application No. 60/358,459, filed on Feb. 19, 2002.

(51) Int. Cl. C12Q 1/68; G01N 33/53; C07K 16/44

(52) U.S. Cl. 435/7.1; 530/387.3; 435/6

(57) ABSTRACT

Methods and compositions for the modulation of an immune response are provided. Compositions comprise a bi-functional surrogate antibody molecule that interacts with a ligand of interest, wherein the bi-functional surrogate antibody further has attached thereto an immunomodulatory agent and/or a transporting agent. The compositions of the invention find use in a method for delivering an immunomodulatory agent to a ligand of interest. Further provided are methods for modulating an immune response in a subject against a ligand of interest. The method comprises administering a therapeutically effective amount of a bi-functional surrogate antibody of the invention. The methods of the invention also find use in improving the clinical outcome of a subject in need of a modulation in the immune response. Methods are further provided for the treatment or prevention of a variety of conditions and/or disorders including cancer, autoimmune diseases, allergies, prions, and various diseases or conditions of bacterial, parasitic, yeast or viral etiology.

### CROSS-REFERENCE RELATED APPLICATIONS

This application is a continuation application of PCT/US03/005000 filed on Feb. 19, 2003, which claims priority to U.S. Provisional Application No. 60/358,459, filed on Feb. 19, 2002, both of which are incorporated herein by reference in their entiriety.

### FIELD OF THE INVENTION

This invention relates to modulating the immune response and transport.

### BACKGROUND OF THE INVENTION

Traditional approaches to vaccine develop have included the use of live attenuated pathogens, whole-killed pathogens, or inactivated toxins. While these methods have been successful at limiting the spread of certain diseases, there have been drawbacks regarding their use. For example, vaccines containing a live pathogen, whether they are an attenuated or related but less virulent version of the virulent strain, are usually highly effective at inducing a full range of immune responses. However, these types of vaccines have the possibility of reversion to a virulent form. In whole-killed vaccines, the primary disadvantage is that the antigen is processed solely as exogenous antigen, and often results in poor cell mediated immunity. More recent approaches in vaccine development include the use of subunit vaccines, synthetic peptides, or plasmid DNA. Although they carry no risk of infection, subunit vaccines and synthetic polypeptides, are poorly immunogenic and have high production costs.

Methods and compositions are needed to effectively and efficiently generate an antigen-specific immune response.

### SUMMARY OF THE INVENTION

Method and compositions are provided for modulating the immune system. Specifically, the present invention provides bi-functional surrogate antibody molecules that interact with a ligand of interest and further have attached thereto an immunomodulatory agent. In this manner, the interaction of the bi-functional surrogate antibody molecule with the ligand of interest allows for a targeted immune response at the site of the ligand/bi-functional surrogate antibody interaction.

The compositions of the invention comprise an isolated bi-functional surrogate antibody molecule comprising a specificity strand and a stabilization strand. The specificity strand comprises a nucleic acid sequence having a specificity region flanked by a first constant region and a second constant region. The stabilization strand comprises a first stabilization domain that interacts with the first constant region and a second stabilization domain that interacts with the second constant region. The bi-functional surrogate antibody further has attached thereto an immunomodulatory agent; and, the bi-functional surrogate antibody molecule is capable of interacting with a ligand of interest.

In other embodiments, the stabilization strand and the specificity strand comprise distinct molecules. In other embodiments, the stabilization strand further comprises a first spacer domain between the first stabilization domain and the second stabilization domain. In other embodiments, the stabilization strand comprises an amino acid sequence or polymer of a nucleic acid binding molecule. In other embodiments, the stabilization strand comprises a second nucleic acid sequence.

The invention further provides an isolated bi-functional surrogate antibody molecule wherein the immunomodulatory agent comprises an immunoglobulin constant region, an active fragment of an immunoglobulin constant region, a variant of an immunoglobulin constant region, an IgG immunoglobulin constant region, a active variant of an IgG immunoglobulin constant region, an active fragment of an IgG immunoglobulin constant region, a cytokine, a variant of the cytokine, an active fragment of the cytokine, a chemokine, an active variant of a chemokine, an active fragment of a chemokine, a CpG motif, an immunostimulatory CpG motif, an adhesion molecule, an active variant of an adhesion molecule, an active fragment of an adhesion molecule, a lipopolysaccharide or an active derivative of a lipopolysaccharide.

In other embodiments, the bi-functional surrogate antibody molecules of the invention are bi-specific antibodies. Thus, the immunomodulatory agent attached to the bi-functional surrogate antibody molecule comprises a second specificity domain, wherein the second specificity region is capable of interacting with an immune response regulator. In one embodiment, the second specificity region interacts with an FγR receptor.

In further embodiments, the isolated bi-functional surrogate antibody molecule interacts with a ligand of interest. A variety of ligands can be used including, for example, a polypeptide, a cell, a prion, or a microbe.

Methods of the invention comprise a method of delivering an immunomodulatory agent to a ligand of interest. The method comprises administering to a subject a composition comprising an isolated bi-functional surrogate antibody molecule wherein the immunomodulatory agent is attached to the bi-functional surrogate antibody molecule; and, the bi-functional surrogate antibody molecule is capable of interacting with the ligand of interest.

Additional methods of the invention include a method for modulating an immune response against a ligand of interest in a subject comprising administering to the subject an isolated bi-functional surrogate antibody molecule wherein said surrogate antibody has attached thereto an immunomodulatory agent; and, the bi-functional surrogate antibody molecule is capable of interacting with the ligand of interest.

Further provided are methods for treating and/or preventing various disorders including, for example, cancers, autoimmune diseases, and various disease and conditions of bacterial, parasitic, yeast, or viral etiology.

Further compositions of the invention include a bi-functional surrogate antibody having attached thereto an transport agent; and, the bi-functional surrogate antibody molecule is capable of interacting with a ligand of interest. In one embodiment, the transport agent comprises the constant region of IgA or an active fragment or variant thereof, or the constant region of IgM or an active fragment or variant thereof.

### BRIEF DESCRIPTION OF THE FIGURES

FIG. 1 is a diagram representing a non-limiting surrogate antibody molecule that contains one or more stabilization regions (ST) composed of two juxtaposed oligonucleotide strands. The lower strand (stabilization strand) comprises a spacer region (S) flanked by two stabilization regions (A′ and D′) that interact with the respective constant region (A and D) of the upper strand (specificity strand). SP designates the specificity region, S designates the spacer domain, and ST designates the stabilization domains. In the present invention, the surrogate antibody further has attached thereto an immunomodulatory agent.

FIGS. 2A, 2B, and 2C are diagrams representing two non-limiting embodiments of a surrogate antibody molecules that include multiple specificity regions (SP), stabilization regions (ST), and spacer regions (S).

FIGS. 3 provides diagrams representing four non-limiting embodiments of surrogate antibody molecules that contain multiple specificity regions (SP), stabilization regions (ST), and spacer regions (S) and that collectively provide multi-dimensional ligand binding.

FIG. 4 is a schematic illustration showing the binding of target ligands to surrogate antibody molecules containing SP region loops of varying sizes.

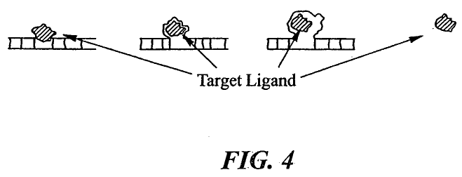

FIG. 5 is a schematic illustration showing surrogate antibody capacity to enhance binding affinity and specificity relative to natural antibodies.

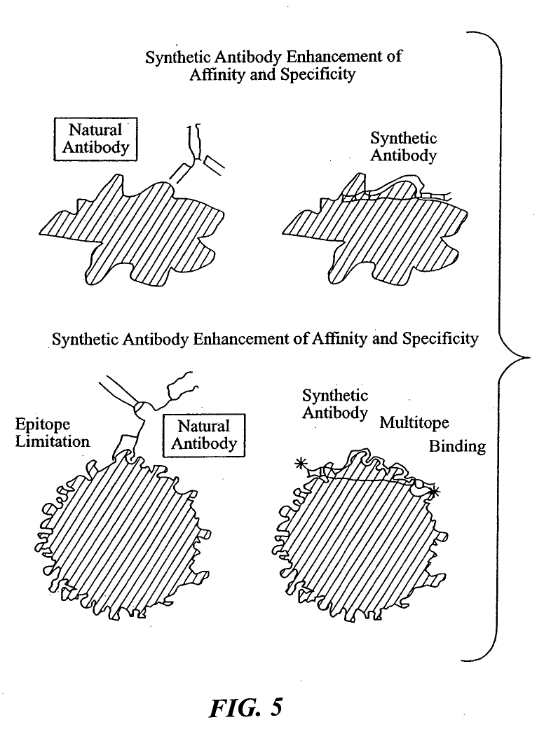

FIG. 6 is a schematic illustration of one method of preparing surrogate antibodies.

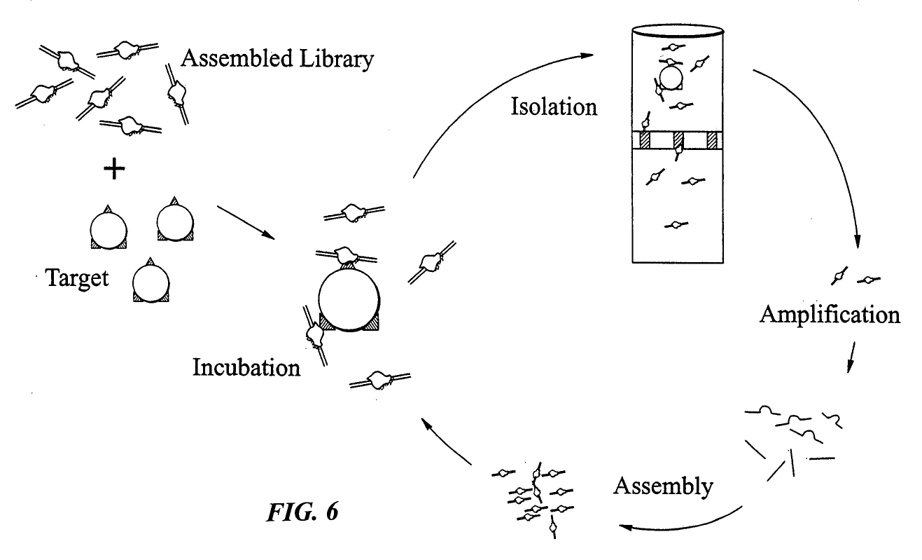

FIG. 7 provides a non-limiting method for amplifying a surrogate antibody. In this embodiment, “F48” comprises the stabilization strand (SEQ ID NO: 1) and “F22-40-25 (87)” comprises the specificity strand (SEQ ID NO: 2). The stabilization strand comprises a 5 nucleotide mis-match (shaded box) to the specificity strand. This mis-match in combination with the appropriate primers (B21-40, SEQ ID NO:3 ; and F1 7-50, SEQ ID NO:4) will prevent amplification of the stabilization strand during PCR amplification. More details regarding this method of amplification are provided elsewhere herein.

FIG. 8 is a schematic view of the 4-chain structure of human IgG1ₖ. The numbers on right side correspond to the actual residue numbers in protein EU (Edelman et al. (1969) Proc. Natl. Acad. Sci. USA 63: 78-85). The numbers on the left half indicate the CDR (complementary-determining segments/regions for the light and heavy chains). Hypervariable regions and complementarity-determining segments or regions (CDR) are represented by heavier lines. V_{L} and V_{H} refer to the light and heavy chain variable region. C_{H}1, C_{H}2, C_{H}3 refer to domains of constant region of heavy chain. C_{L} refers to the constant region of light chain. Hinge region in which two heavy chains are linked by disulfide bonds is indicated approximately. Attachment of carbohydrate is at residue 297 is shown. Arrows at residues 107 and 110 denote transition from variable to constant regions. Sites of action of papain and pepsin and locations of a number of genetic factors are given.

FIG. 9 is a non-denaturing acrylamide gel that verifies the duplex nature of the surrogate antibody molecules.

FIG. 10 is a denaturing acrylamide gel that verifies the duplex nature of the surrogate antibody molecules.

FIG. 11 illustrates the selection and enrichment of the surrogate antibodies to BSA-PCB (BZ11 congener) conjugates. Signal/Negative control represents as a percent, the amount of surrogate antibody bound to the target verses the amount of surrogate antibody recovered when the target is absent (negative control).

FIG. 12 illustrates the selection and enrichment of the surrogate antibodies to IgG. Signal/Negative control represents as a percent, the amount of surrogate antibody bound to the target verses the amount of surrogate antibody recovered when the target is absent (negative control).

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Overview

While the binding of an antibody to the requisite antigen has a neutralizing effect that might prevent the binding of a foreign antigen to its endogenous target (e.g. receptor or ligand), binding alone may not remove the foreign antigen. To be efficient in removing and/or destroying foreign antigens, an antibody should be endowed with both high affinity and specificity binding to its target antigen and efficient immune effector functions. The present invention is directed to compositions and methods comprising a bi-functional surrogate antibody molecule and various populations of bi-functional surrogate antibody molecules. As used herein, a “bi-functional” surrogate antibody refers to a class of molecules that contain discrete nucleic acid structures or motifs that enable selective binding to a ligand of interest and further have attached thereto an immunomodulatory agent and/or transporting agent. In this manner, interaction of the bi-functional surrogate antibody molecule with the ligand of interest allows for a targeted modulation of the immune response at the site of the ligand/surrogate antibody interaction.

The bi-functional surrogate antibody molecules of the invention “modulate an immune response. By “modulate” or “modulation” is intended an increase or a decrease in a particular character, quality, activity, substance, or response. For example, the modulation in the immune response could comprise an increase or decrease in antibody-dependant cell-mediated cytotoxicity (ADCC), phagocytosis, complement-dependent cytotoxicity (CDC), half-life/clearance rate, dependant cell cytotoxicity, opsonin induced phagocytosis, complement-dependant lysis, cytotoxic T-cell (CTL) killing, polymorphonuclear (PMN) cell killing, immediate type hypersensitivity, and delayed type hypersensitivity. Thus, the bi-functional surrogate antibodies of the invention are designed for the recruitment of the immune system to the site of the ligand of interest. Depending on the desired modulation of immune response (i.e., antibody-dependant cytotoxicity (ADCC), phagocytosis, complement-dependent cytotoxicity (CDC), and half-life/clearance rate), the appropriate immunomodulatory agent is attached to the bi-functional surrogate antibody molecule.

For example, if immune system recruitment is desired, the bi-functional surrogate antibody molecule can comprise an immunomodulatory agent able to improve immune effector function at the site of the ligand of interest. In this instance, the immunoglobulin G (IgG) Fc portion could be attached to the bi-functional surrogate antibody molecule and thereby potentiate immune effector function through improved binding to FcγR and/or complement activation. In other embodiments, if immune effector functions are deleterious but a long half-life is desired, an immunoglobulin constant region or an engineered immunoglobulin that increases the half-life of the molecule could be attached to the bi-functional surrogate antibody. Further details regarding immunomodulatory agents of interest are provided elsewhere herein. Accordingly, bi-functional surrogate antibody molecules of the invention can be designed to have the desired therapeutic activity (i.e., the desired binding affinity and specificity to the ligands of interest and the desired immune effector functions for the intended application).

The compositions of the invention find use in a method for delivering an immunomodulatory agent to a ligand of interest. The compositions of the invention find further use in modulating an immune response in a subject against a ligand of interest. The method comprises administering to a subject a therapeutically effective amount of a bi-functional surrogate antibody of the invention.

The compositions and methods of the invention find further use as therapeutic bi-functional surrogate antibodies that can be used to treat or prevent a variety of conditions. Thus, the methods of the invention find use in improving the clinical outcome of a subject in need of a targeted immune response. By “treatment or prevention” is intended obtaining a desired pharmacologic and/or physiological effect. The effect may be prophylactic in terms of completely or partially preventing a particular infection or disease or sign or symptom thereof and/or may be therapeutic in terms of a partial or complete cure of an infection or disease and/or adverse effect attributable to the infection or disease. Accordingly, the method of the invention “prevents” (i.e., delays or inhibits) and/or “reduces” (i.e., decreases, slows, or ameliorates) the detrimental effects of a disease or disorder in the subject receiving the bi-functional surrogate antibody molecule. The subject may be any animal, preferably a mammal, including a human, pig, cow, moose, rat, sheep, horse, dog, cat, avian, chicken, for example.

In further compositions of the invention, the bi-functional surrogate antibody comprises a transport agent. As discussed below, the transport agent mediates transcytosis and thereby allows the delivery of the surrogate antibody to mucosal lining.

As discussed in further detail below, the bi-functional surrogate antibodies of the invention and various populations of bi-functional surrogate antibodies (i.e., selected populations, polyclonal populations, and monoclonal bi-functional surrogate antibody populations) can be generated that interact with a desired ligand of interest. As such, the bi-functional surrogate antibody provides a targeted modulation in the immune response at the site of the desired ligand. Thus, the bi-functional surrogate antibodies can be used to replace conventional antibodies in testing, pharmaceutical, and research applications.

As used herein, “ligand” can be any molecule of interest that interacts with the bi-functional surrogate antibody, including, but not limited to, an ion, a molecule, or a molecular group. As used herein, the ligand need not be antigenic. Thus, the ligand can be a cell and/or any of the cell's constituents or immunological hapten. The ligand can be any cell type of interest, at any developmental stage, and having various phenotypes and in various pathological states (i.e., normal and abnormal states). For example, the bi-functional surrogate antibodies can be developed to bind ligands comprising normal, abnormal, and/or unique constituents found on or within a microbe (i.e., prokaryotic cells (e.g. bacteria), viruses, fungi, protozoa, and parasites) or on or within a eukaryotic cell (e.g. epithelial cells, muscle cells, nerve cells, sensory cells, cancerous cells, secretory cells, malignant cells, erythroid and lymphoid cells, stem cells, ect.). Ligands of interest may also include one or more constituents of a cell type described above.

For example, the ligand of interest used to develop the bi-functional surrogate antibody of the invention can comprise a variety of tumor cells, such as melanoma cells, colon tumor cells, breast cancer cells, breast tumor cells, prostate tumor cells, glioblastoma cells, renal carcinoma cells, neuroblastoma cells, lung cancer cells, bladder carcinoma cells, plasmacytoma colon cancer cells, breast cancer cells, lymphoma cells and/or various constituents of the cell types. Such ligands can be obtained from culturing resected tumors or from established cell lines (i.e., human cell lines) such as HCT 116, Colo205, SW403 or SW620 (for colon cancer) and BT-20 cell line (for breast cancer). Such cells are available to one skilled in the art, for example, from the American Type Culture Collection (ATCC; Rockville, Md.). In addition, the ligand of interest may be primary glioma cells or cells from established human glioblastoma or astrocytoma lines. Primary cultures of glioma cells can be established from surgically resected tumor tissue as described in Wakimoto et al. (1999) Japan. J Cancer Res. 88:296-305 (1997), which is incorporated herein by reference. Human glioblastoma cell lines, such as U-87 MG or U-1 18 MG, or human astrocytoma lines, such as CCF-STTG1 or SW1088 (Chi et al. (1997) Amer. J. Path. 150:2142-2152) can be obtained from ATCC. Additional types of undesirable cells that can be used as ligands in the present invention include auto-antibody producing lymphocytes, for the treatment of an autoimmune disease, or an IgE producing lymphocyte for the treatment of an allergy.

Further, while the ligand of interest need not be antigenic, in some embodiments, the ligand can be a disease-associated antigen including, for example, tumor-associated antigens and autoimmune disease-associated antigens. Such disease-associated antigens are known in the art and include, for example, i.e., growth factor receptors, cell cycle regulators, angiogenic factors, and signaling factors.

Other ligands of interest include, an organic compound, an inorganic molecule, a toxic environmental compound, a nucleic acid, a protein, a polypeptide, a glycoprotein, a receptor, a growth factor, a hormone, an enzyme, natural and synthetic polymers, a carbohydrate, a polysaccharide, a mucopolysaccharide, an effector, an antigen, an antibody, a prion, a substrate, a metabolite, a immunological hapten or small molecule, a drug, a toxin, a transition state analog, a cofactor, an inhibitor, a nutrient, a unique cell surface determinant or intracellular marker, etc., without limitation. Ligands can further include organic or inorganic environmental pollutants (e.g., PCBs, dioxins, petroleum hydrocarbons), immunological haptens including therapeutic drugs and substances of abuse.

The bi-functional surrogate antibodies of the present invention interact with a desired ligand and are also designed to modulate an immune response. As such, the bi-functional surrogate antibodies can be used to treat or prevent a variety of conditions/disorders including, but not limited to, tumors and cancers, autoimmune diseases, infectious diseases and disorders of bacterial, parasitic or viral etiology. In one embodiment, the methods of the invention can be used to modulate an immune response for protection against or treatment of cancer, including cancers such as melanoma, colorectal cancer, prostate cancer, breast cancer, ovarian cancer, cervical cancer, endometrial cancer, glioblastoma, renal cancer, bladder cancer, gastric cancer, pancreatic cancer, neuroblastoma, lung cancer, leukemia and lymphoma. The methods of the invention also can be used to protect against or treat infectious diseases such as Acquired Immunodeficiency Syndrome (AIDS).

In addition, the methods of the invention can be used to protect against the development of or to treat existing autoimmune diseases such as rheumatoid arthritis, psoriasis, multiple sclerosis, systemic lupus erythematosus and Hashimoto's disease, type I diabetes mellitus, myasthenia gravis, Addison's disease, autoimmune gastritis, Graves' disease and vitiligo. Allergic reactions, such as hay fever, asthma, systemic anaphylaxis or contact dermatitis also can be treated using the methods of the invention for modulating an immune response.

A variety of diseases or conditions of bacterial, parasitic, yeast or viral etiology also can be prevented and treated using the methods of the invention. Such diseases and conditions include gastritis and peptic ulcer disease; periodontal disease; Candida infections; helminthic infections; tuberculosis; Hemophilus-mediated disease such as bacterial meningitis; pertussis virus-mediated diseases such as whooping cough; cholera; malaria; influenza infections; respiratory syncytial antigens; hepatitis; poliomyelitis; genital and non-genital herpes simplex virus infections; rotavirus-mediated conditions such as acute infantile gastroenteritis and diarrhea; and flavivirus-mediated diseases such as yellow fever and encephalitis. In addition, the methods and compositions of the invention find use in treating exposure to biowarfare agents including, but not limited to, (e.g., Clostridium toxins, hemorrhagic fever viruses, and bacteria such as Francisella tularensis, Yersinia pestis, and Bacillus antracsis).

As disclosed herein, the methods of the invention can be used to treat an individual having one of such diseases or conditions or an individual suspected of having one of such diseases or conditions. The methods of the invention also can be used to protect an individual who is at risk for developing one of such diseases or conditions from the development of the actual disease. Individuals that are predisposed to developing particular diseases, such as particular types of cancer, can be identified using methods of genetic screening. See, for example, Mao et al. (1994) Canc. Res. 54(Suppl.):1939s-1940s and Garber et al. (1993) Curr. Opin. Pediatr. 5:712-715, each of which is incorporated herein by reference. Such individuals can be predisposed to developing, for example, melanoma, retinoblastoma, breast cancer or colon cancer or disposed to developing multiple sclerosis or rheumatoid arthritis.

Compositions

I. Bi-Functional Surrogate Antibodies

The bi-functional surrogate antibodies of the present invention comprise diverse structures that allow for the development of antibodies having a diverse range of binding specificities and binding affinities to the ligand of interest. Details regarding these diverse structures and how the bi-functional surrogate antibodies of the invention are developed are described in more detail below.

The bi-functional surrogate antibody comprises a first strand, referred to herein as the “specificity strand” and a second strand referred to herein as the “stabilization strand”. The specificity strand comprises a nucleic acid sequence having a specificity region flanked by a first constant region and a second constant region. The stabilization strand comprises a first stabilization region that interacts with the first constant region and a second stabilization region that interacts with the second constant region. Such surrogate antibody molecules are further described in U.S. Provisional Application No. 60/358,459, filed Feb. 19, 2002 and U.S. Utility Application entitled “Surrogate Antibodies and Methods of Preparation and Uses Thereof”, filed concurrently herewith. The bi-functional surrogate antibody molecule of the invention further has attached thereto an immunomodulatory agent that is capable of modulating an immune response.

The invention encompasses isolated or substantially isolated bi-functional surrogate antibody compositions. An “isolated” bi-functional surrogate antibody molecule is substantially free of other cellular material, or culture medium, chemical precursors, or other chemicals when chemically synthesized. A bi-functional surrogate antibody that is substantially free of cellular material includes preparations of surrogate antibody having less than about 30%, 20%, 10%, 5%, (by dry weight) of contaminating protein or nucleic acid. In addition, if the surrogate antibody molecule comprises nucleic acid sequences homologous to sequences in nature, the “isolated” bi-functional surrogate antibody molecule is free of sequences that may naturally flank the nucleic acid (i.e., sequences located at the 5′ and 3′ ends of the nucleic acid) in the genomic DNA of the organism from which the surrogate antibody has homology.

As used herein, nucleic acid means TNA, DNA, RNA, single-stranded or double-stranded, and any chemical modifications thereof. A bi-functional surrogate antibody can be composed of double-stranded RNA, single-stranded RNA, single stranded DNA, double stranded DNA, a hybrid RNA-DNA double strand combination, a hybrid TNA-DNA, a hybrid TNA-RNA, a hybrid amino acid/RNA, amino acid/DNA, or amino acid/TNA combination provided there exists interacting constant domains that allow for the stabilization of one or more specificity domains. It is further recognized that the nucleotide or amino acid residues can include naturally occurring residues and/or synthetically modified residues.

A. The Specificity Strand

As used herein, the specificity strand of the bi-functional surrogate antibody comprises a nucleic acid molecule having a specificity region flanked by two constant regions. As used herein, “flanked by” is intended the constant regions are immediately adjacent to the specificity region or, alternatively, the constant regions are found 5′ and 3′ to the specificity region but separated by a spacer sequence. The specificity region functions as a ligand binding site, while the constant domains interact with the stabilization domains found on the stabilization strand to thereby allow the specificity domain to form a region that interacts with the ligand of interest.

The specificity strand comprises a nucleic acid sequence composed of ribonucleotides, modified ribonucleotides, deoxyribonucleotides, modified deoxyribonucleotides, (3′,2′)-α-L-threose nucleic acid (TNA), modified TNA, or any combination thereof. See, Chaput et al. (2003) J. Am. Chem. Soc. 125:856-857, herein incorporated by reference. A modification includes the attachment of any functional moiety or molecule to the nucleotide sequence. The modification can be at the 5′ end and/or the 3′ end of the sequence, added to individual nucleotide residues anywhere in the strand, attached to all or a portion of the pyrimidines or purine residues, or attached to all or a portions of a given type of nucleotide residue. While various modifications to DNA and RNA residues are known in the art, examples of some modifications of interest to the bi-functional surrogate antibodies of the present invention are discussed in further detail below.

The specificity strand and its respective domains (i.e., the constant domains and the specificity domains and, in some embodiments, a spacer regions) can be of any length, so long as the strand can form a bi-functional surrogate antibody as described elsewhere herein. For example, the specificity strand can be between about 10, 50, 100, 200, 400, 500, 800, 1000, 2000, 4000, 8000 nucleotides or greater. Alternatively, the specificity strand can be from about 15-80, 80-150, 150-600, 600-1200, 1200-1800, 1800-3000, 3000-5000 or greater nucleotides. The constant domains and the specificity domains can be between about 2 nucleotides to about 100 nucleotides in length, between about 20 to about 50 nucleotides in length, about 10 to about 90 nucleotides in length, about 10 to about 80 nucleotides in length, about 10 to about 60 nucleotides in length, or about 10 to about 40 nucleotides in length.

While a bi-functional surrogate antibody molecule does not require a spacer region in the specificity strand, if the region is present it can be of any length. For example, if a spacer region is present in the specificity strand, this region can be about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. In yet other embodiments, the spacer region need not comprise a nucleic acid residue but could comprise any molecule, such as a phosphate moiety, incorporated into the strand that provides the desired spacing to form the bi-functional surrogate antibody molecule.

In some embodiments, the specificity strand or its components (the constant regions or the specificity region) have significant similarity to naturally occurring nucleic acid sequences. In other embodiments, the nucleic acid sequence can share little or no sequence identity to sequences in nature. In still other embodiments, the nucleic acid residues may be modified as described elsewhere herein.

B. The Stabilization Strand

The bi-functional surrogate antibody further comprises a stabilization strand. The stabilization strand comprises any molecule that is capable of interacting with the constant domains of the specificity strand and thereby stabilize the ligand-binding cavity of the specificity domain. Accordingly, the stabilization strand can comprise, for example, an amino acid sequence, a nucleic acid sequence, or various polymers including any cationic polymer, a cyclodextrin polymer, or a polymer having an appropriately charged intercalating agent, such as lithium bromide or ethidium bromide.

It is recognized that the stabilization regions in a bi-functional surrogate antibody can be identical (i.e., the same nucleotide sequence or peptide sequence) or the regions can be non-identical, so long as each stabilization region interacts with their corresponding constant region found in the specificity strand. In addition, the interaction between the constant regions and the stabilization regions may be direct or indirect. The interaction will further be such as to allow the interaction to occur under a variety of conditions including under physiological conditions (i.e. the desired ligand binding conditions).

In some embodiments, the stabilization strand and the specificity strand and/or their respective domains are not naturally occurring in nature. In others embodiments, they can have significant similarity to a naturally occurring nucleic acid sequences or amino acid sequences or may actually be naturally occurring sequences. One of skill in the art will recognize that the length of the stabilization domain will vary depending on the type of interaction required with the constant domains of the specificity strand. Such interactions are discussed in further detail elsewhere herein.

A stabilization strand comprising an amino acid sequence may comprise any polypeptide that is capable of interacting with the nucleic acid sequence of the constant domains of the specificity strand. For example, amino acid sequences having DNA binding activity (i.e., zinc finger binding domains (Balgth et al. (2001) Proc. Natl. Acad. Sci. 98:7158-7163; Friesen et al. (1998) Nature Structural Biology; Tang et al. (2001) J. Biol. Chem. 276:19631-9; Dreier et al. (2001) J. Biol. Chem. 29466-79; and Sera et al. (2002) Biochemistry 41:7074-81, helix-turn domains, leucine zipper motifs (Mitra et al. (2001) Biochemistry 40:1693-9) or polypeptides having lectin-activity may be used for one or more of the stabilization domains. Accordingly, various polypeptides could be used, including transcription factors, restriction enzymes, telomerases, RNA or DNA polymerases, inducers/repressors or fragments and variants thereof that retain nucleic acid binding activity. See for example, Gadgil et al.(2001) J. Biochem. Biophys. Methods 49: 607-24. In still other embodiments, the stabilization strand can include sequence-specific DNA binding small molecules such as polyamides (Dervan et al. (1999) Current Opinion Chem. Biol. 6:688-93 and Winters et al. (2000) Curr Opin Mol Ther 6:670-81); antibiotics such as aminoglycosides (Yoshhizawa et al. (2002) Biochemistry 41:6263-70) and quinoxaline antibiotics (Bailly et al.(1998) Biochem Inorg Chem 37:6874-6883; AT-specific binding molecules (Wagnarocoski et al. (2002) Biochem Biophys Acta 1587:300-8); and rhodium complexes (Terbrueggen et al. (1998) Inorg. Chem. 330:81-7).

One of skill in the art will recognize that if, for example, a zinc finger binding domain is used in the stabilization strand, the corresponding nucleic acid binding site will be present in the desired constant region of the specificity strand. Likewise, if a polypeptide having lectin-activity is used in the stabilization strand, the corresponding constant domain of the specificity strand will have the necessary modifications to allow for the desired interaction. When the stabilization domain comprises an amino acid sequence, any of the amino acid residues can be modified to contain functional moieties. Such modifications are discussed in further detail elsewhere herein.

When the stabilization strand comprises a nucleic acid molecule, the bi-functional surrogate antibodies comprise a nucleotide sequence comprising a specificity strand, which as describe above, comprises two constant regions that are complementary to the two stabilization regions on the stabilization strand. In this embodiment, the bi-functional surrogate antibodies are formed when the stabilization strand and the specificity strand are hybridized together to allow for the appropriate interaction between the stabilization domains and the constant domains. In one embodiment, the stabilization strand is longer than the specificity strand.

The stabilization strand can comprise any nucleotide base, including for example, ribonucleotides, modified ribonucleotides, deoxyribonucleotides, modified deoxyribonucleotides or any combination thereof.

C. Forming a Bi-Functional Surrogate Antibody

Methods of forming a bi-functional surrogate antibody molecule comprise providing a specificity strand and a stabilization strand and contacting the specificity strand and the stabilization strand under conditions that allow for the first stabilization domain to interact with the first constant region and the second stabilization domain to interact with the second constant region. The specificity strand and stabilization strand can be contacting under any condition that allows for the stable interaction of the stabilization domains and the constant domains. This method of forming a surrogate antibody can be used to generate a population of surrogate antibodies.

In preferred embodiments, the bi-functional surrogate antibody molecule is formed under physiological conditions. One of skill will be able to empirically determine the appropriate conditions for the intended application. For example, the physiological conditions can comprise a pH of about 6.5 to about 8.0, about 7.0 to about 7.6, or a pH of about 7.2, 7.3, 7.4, or 7.5. Physiological conditions comprise physiological salt conditions of about 230 to about 350 milliosmols, about 250 to about 300 milliosmols, about 280 milliosmols to about 300 milliosmols. Alternatively, the physiological salt conditions can comprise about 270 milliosmols, 280 milliosmols, 290 milliosmols, 300 milliosmols, 310 milliosmols, 330 milliosmols, 340 milliosmols, 350 milliosmols, 360 milliosmols, 370 milliosmols or 380 milliosmols. Physiological conditions further comprise a temperature of about 34° C. to about 39° C. and about 35° C. to about 38° C., about 36° C. to about 37° C. One of skill will be able to determine the appropriate salt concentration and pH for the intended application. In one embodiment, physiological conditions comprise a pH of 7.4 and a salt concentration of 280 to about 300 milliosmols at about 37° C.

When the stabilization strand comprises a nucleic acid sequence, the nucleotide sequences of the constant regions and the stabilization regions will be such as to allow for an interaction (i.e., hybridization) under the desired conditions (i.e., physiological conditions). Furthermore, the design of each stabilization domain and each constant domain will be such as to allow for assembly such that the first constant domain preferably interacts with the first stabilization domain and the second stabilization domain preferably interacts with the second constant domain. In this way, upon the interaction of the specificity strand and stabilization strand, sequence directed self-assembly of the bi-functional surrogate antibody can occur.

In one embodiment, the surrogate antibody molecule is designed to result in a Tm for of each stabilization/constant domain interaction to be approximately about 15 to about 25° C. above the temperatures of the intended application (i.e., the desired ligand binding conditions). Accordingly, if the intended application is a therapeutic application or any application performed under physiological conditions, the Tm can be about 37° C+about 15° C. to about 37° C.+25° C. (i.e., 49° C., 50° C., 52° C., 60° C., 62° C., 64° C., or greater). If the intended application is a diagnostic assay conducted at room temperature, the Tm can be 25° C.+about 15° C. to about 25° C.+about 25° C. (i.e.,38° C., 40° C., 41° C., 42° C., 43° C., 44° C., 46° C., 48° C., 50° C., 52° C., 53° C. or greater). Equations to measure Tm are known in the art. A preferred program for calculating Tm comprises the OligoAnalyzer 3.0 from IDT BioTools @ 2000. It is recognized that any temperature can be used the methods of the invention. Thus, the temperature of the ligand binding conditions can be about 5° C., 10° C., 15° C., 16° C., 18° C., 20° C., 22° C., 24° C., 26° C., 28° C., 30° C., 32° C., 34° C., 38° C., 40° C., 42° C., 44° C., 46° C., 48° C., 50° C., 52° C., 54° C., 56° C., 58° C., 60° C. or greater.

Alternatively, the stabilization domains and the respective constant domains are designed to allow about 40% to about 99%, about 40% to about 50%, or about 50% to about 60%, about 60% to about 70%, about 70% to about 80%, about 85%, about 90%, about 95%, about 98% or more of the surrogate antibody population to remain annealed under the intended ligand binding conditions. Various methods, including gel electrophoresis, can be used to determine the % formation of the surrogate antibody. See Experimental section. In addition, calculation for this type of determination can be found, for example, in Markey et al. (1987) Biopolymers 26:1601-1620 and Petersteim et al. (1983) Biochemistry 22:256-263, both of which are herein incorporated by reference.

The relative concentration of the specificity strand and the stabilization strand can vary so long as the ratio will favor the formation of the bi-functional surrogate antibody. Such conditions include providing an excess of the stabilization strand.

When the stabilization strand and the specificity strand are nucleic acid molecules, the constant regions and stabilization regions can have any desired G/C content, including for example, about 0%, 5%, 10%, 15%, 20%, 25%, 30%, 35%, 40%, 50%, 55%, 60%, 65%, 70%, 75%, 80%, 85%, 90%, 95%, or 100% G/C.

The stabilization strand and the domains contained therein (stabilization domains and, in some embodiments, spacer domains) can be of any length, so long as the strand can form a surrogate antibody as described herein. For example, the stabilization strand can be between about 8, 10, 50, 100, 200, 400, 500, 800, 1000, 2000, 4000, 8000 nucleotides or greater in length. Alternatively, the stabilization strand can be form about 15-80, 80-150, 150-600, 600-1200, 1200-1800, 1800-3000, 3000-5000 nucleotides or greater.

The stabilization domains can be between about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. If a spacer region is present in the stabilization strand, this region can be about 1 nucleotides to about 100 nucleotides in length, between about 5 to about 50 nucleotides in length, about 10 to about 90 nucleotides in length, about 10 to about 60 nucleotides in length, or about 10 to about 40 nucleotides in length. Alternatively, as discussed elsewhere herein, the spacer can comprise one or more molecules including, for example, a phosphate moiety. The length and G/C content of each domain can vary so long as the interaction between the constant domains and the stabilization domain is sufficient to stabilize the surrogate antibody structure and produce a stable specificity region. In addition, the stabilization strand can be linear, circular, or globular and can further comprise stabilization domains that allow for multiple (2, 3, 4, 5, 6, or more) specificity strands to interact.

One of skill in the art will recognize that the stabilization strand stabilization domains and specificity strand constant domains can be designed to maximize stability of the interactions under the desired conditions and thereby maintain the structure of the surrogate antibody. See, for example, Guo et al. (2002) Nature Structural Biology 9:855-861 and Nair et al. (2000) Nucleic Acid Research 28:1935-1940. Methods to measure the stability or structure of the surrogate antibody molecules are known. For example, surface plasmon resonance (BIACORE) can be used to determine kinetic values for the formation of surrogate antibody molecules (BIACORE AB). Other techniques of use include NMR spectroscopy and electrophoretic mobility shift assays. See, Nair et al. (2000) Nucleic Acid Research 9:1935-1940. It is recognized that when the stabilization strand and the specificity strand are nucleic acids, the complementary hybridizing stabilization regions and constant regions need not have 100% homology with one another. All that is required is that they interact together in a directed fashion and form a stable structure when exposed to ligand-binding conditions. Generally, this requires a stabilization domain and a constant domain having at least 80% sequence homology, at least 90%, 95%, 96%, 97%, or 98% and higher sequence homology. In addition, the interaction may further require at least 5 consecutive complementary nucleotide residues in the stabilization domain and the corresponding constant domain.

By “sequence identity or homology” is intended that nucleotides with complimenting bases are found within the constant regions and the stabilization domain when a specified, contiguous segment of the nucleotide sequence of the constant domain is aligned and compared to the nucleotide sequence of the stabilization domain. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5:Suppl. 3 (National Biomedical Research Foundation, Washington, D.C.). With respect to optimal alignment of two nucleotide sequences, the contiguous segment of the constant/stabilization domain may have additional nucleotides or deleted nucleotides with respect to the corresponding constant/stabilization nucleotide sequence. The contiguous segment used for comparison to the reference nucleotide sequence will comprise at least 5, 10, 15, 20, 25 contiguous nucleotides and may be 30, 40, 50, 100, or more nucleotides. Corrections for increased sequence identity associated with inclusion of gaps in the nucleotide sequence can be made by assigning gap penalties.

The determination of percent identity between two sequences can be accomplished using a mathematical algorithm. Percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

When the specificity strand and the stabilization strand of the surrogate antibody comprise nucleic acid sequences, the surrogate antibodies can be formed by placing the first and second strand in solution, heating the solution, and cooling the solution under conditions such that, upon cooling, the first and second strand anneal and form the antibody. In other embodiments, the surrogate antibody may be formed without heating.

D. Diverse Structures of Bi-Functional Surrogate Antibodies

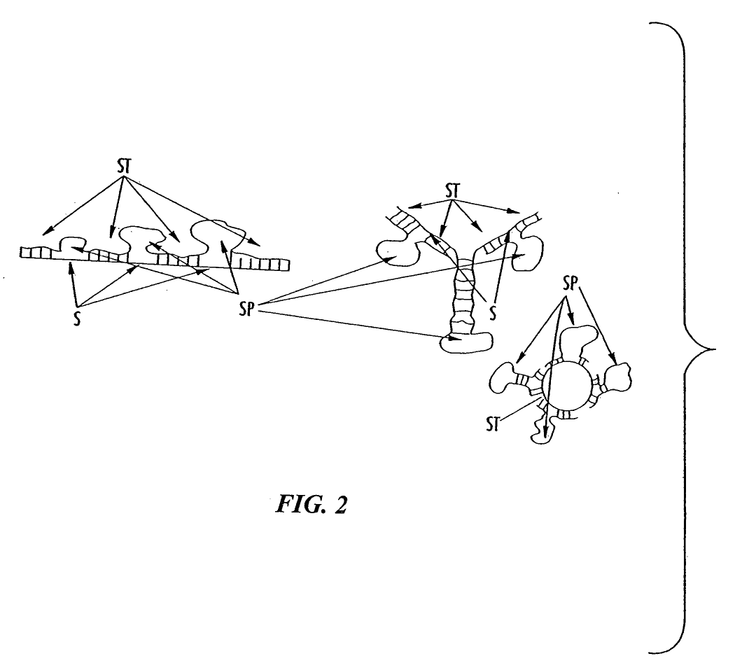

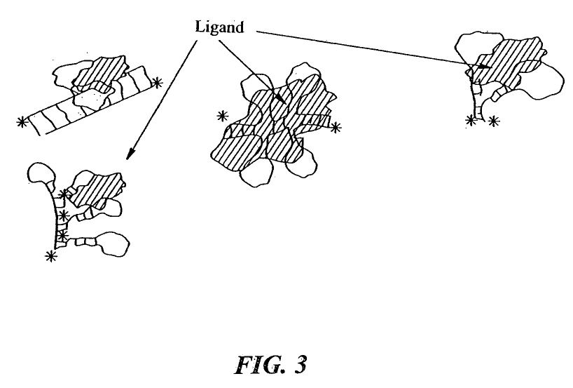

A diverse number of bi-functional surrogate antibodies structures can be formed. In one embodiment, the bi-functional surrogate antibodies described herein can include one or more distinct specificity strands having one or more than one specificity domains, wherein each specificity domain is flanked by constant domains. Bi-functional surrogate antibodies of the invention can therefore have 1, 2, 3, 4, 5 or more specificity domains. Thus, the bi-functional surrogate antibody molecules can be formed using multiple oligonucleotides. See, for example, FIGS. 2 and 3. Accordingly, the bi-functional surrogate antibody can be “multi-valent” and thereby contain multiple specificity domains contained on one specificity strand or on multiple distinct strands. Thus, the specificity domains of a multi-valent surrogate antibody can be the same nucleotide sequence and of the same size and recognize the same ligand binding site. In other embodiments, the specificity domains can be different and thus form “pluri-specific” surrogate antibodies. The pluri-specific antibody will bind to a different ligands or different regions of the same ligand. Accordingly, each specificity domain can be designed to bind the same ligand or to a different ligand. In this way, a bi-surrogate antibody can simultaneously bind two common determinates on a single cell, bind different determinants, or be able to bind a compound in two distinct orientations. For example, an antibody can bind a particular receptor in a preferred binding site and also in an allosteric position. Alternatively, the surrogate antibody can bind a particular pair of receptors on a given cell surface thereby increasing affinity through cooperative binding interactions or form a bridge between molecules or cells.

The bi-functional surrogate antibodies can further contain hinge regions (or spacer regions) between the separate loop structures. The surrogate antibodies can include a “hinge unit” or spacer that functions in a similar manner as hinge units in conventional antibodies. Spacer sequences can be present between the structures on the specificity strand and/or between the stabilization domains of the stabilization strand to sterically optimize binding. In this way, the spacer region can be used to eliminate bond stress in molecules, provide diversity to the size and/or shape of the binding cavity, alter specificity loop orientation, optimize agglutination or flocculation, or optimize energy (Fluor) transfer reactions. Accordingly, the bi-functional surrogate antibody molecule can comprises multiple spacer regions having a common number of nucleotides and nucleotide sequence or a different number of nucleotides and nucleotide sequence.

It is further recognized that when the stabilization strand and the specificity strand comprise a nucleotide sequence, the strands can be contained on the same or distinct, (i.e., different) nucleic acid molecules. Thus, in another embodiment, the surrogate antibodies are formed from a single strand of nucleotides comprising a first constant region, a specificity domain, a second constant region, a second stabilization region that is capable of hybridizing to the second constant region, and a first stabilization region that is capable of hybridizing to the first constant region. In one embodiment, each region contains between about one to about twenty nucleotides and the molecules may further comprise spacer regions to allow for the formation of the surrogate antibody structure. In addition, the strand of nucleotides can be linear or cyclic, so long as the stabilization regions and the constant regions are capable of interacting.

Alternatively, the specificity strands and stabilization strands need not be linked by a covalent interaction. Instead, the specificity strands and stabilization strands can comprise distinct molecules that interact (directly or indirectly) via non-covalent interactions. In this manner, when the specificity strand and the stabilization strand comprise nucleic acid sequences, each “distinct” strand will comprises a nucleic acid sequence having a 3′ and 5′ termini. Accordingly, the invention relates to a ligand-binding surrogate antibody molecule comprising an assembly of two or more single stranded RNA oligonucleotide strands, two or more single stranded DNA oligonucleotide strands, two or more TNA oligonucleotide strands, or a combination of two or more single stranded RNA, DNA, or TNA strands.

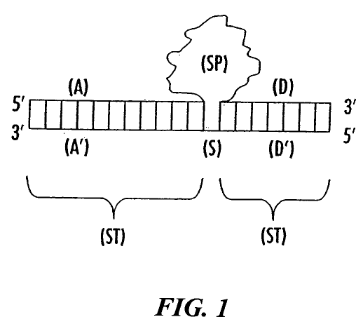

Representations of various types of surrogate antibody molecules are shown in FIGS. 1, 2 and 3. FIG. 2 shows two embodiments of surrogate antibody molecules that include multiple specificity regions. In one embodiment, the surrogate antibody molecules include multiple specificity regions, stabilization regions and spacer regions that collectively provide multi-dimensional ligand binding. These types of molecules are shown, for example, in FIGS. 3a-3d.

E. Immunomodulatory Agents

The bi-functional surrogate antibodies of the invention interact with a desired ligand of interest and further have attached thereto an immunomodulatory agent. By “immunomodulatory agent ” is intended any molecule that is capable of modulating (stimulating or suppressing) an immune response. As discussed below, the modulation of the immune response may be either a direct or an indirect effect.

By “attachment” or “attached” is intended any association (covalent, ionic, hydrophobic, or any other means) of an agent with the bi-functional surrogate antibody. The attachment will be such as to maintain the interaction of the bi-functional surrogate antibody and the immunomodulatory agent under the desired application conditions. Various methods of non-covalent attachment include, for example, avidin-biotin, pre-complexed antibody to conjugated protein, lectin-sugar, clathrating agent such as cyclodextrin bound to coupled compounds ect. The immunomodulatory agent can be attached to any region of the surrogate antibody (i.e., the stabilization strand, at least one stabilization domains, the specificity strand, the specificity domain, at least one constant domain, and if present the spacer domain or any combination thereof.

The attachment of the immunomodulatory agent can occur at any location (i.e., residue) on the surrogate antibody. “Attachment” to a nucleic acid sequence therefore encompasses covalent linking to, for example, the sugar group or, alternatively, if the immunomodulatory agent is also a nucleic acid sequence (i.e., a CpG motif), the agent can be attached via a phosphate linkage either internally in the strand or at the 5′ or 3′ termini. Similarly, when the stabilization strand comprises an amino acid sequence the attachment of the immunomodulatory agent can occur at any residue. In some embodiments, the attachment occurs at the N- or C- terminus of the stabilization strand.

Various methods for attaching the immunomodulatory agent to the surrogate antibody structure are known in the art. For example, bioconjugation reactions that provide for the conjugation of polypeptides or various other compounds of interest to the surrogate antibody can be found, for example, in Aslam et al. (1999) Protein Coupling Techniques for Biomed Sciences, Macmillan Press; Solulink Bioconjugation systems at www.solulink.com; Sebestyen et al. (1998) Nature Biotechnology 16:80-85; Soukchareum et al. (1995) Bioconjugate chem. 6:43-54; Lemaitre et al. (1987) Proc. Natl Acad Sci USA 84:648-52 and Wong et al. (2000) Chemistry of Protein Conjugation and Cross-Linking, CRC, all of which are herein incorporated by reference.

One or more of the same or different immunomodulatory agents can be attached to one or more of the strands that form the bi-functional surrogate antibodies. The strands of the surrogate antibody molecule can be attached to one, two, three, four or more different or identical immunomodulatory agents. The agents can be at either or both of the terminal ends of either the stabilization strand or the specificity strand, added to individual residues anywhere in either strand, attached to all or a portion of the residues, or attached to all or a portions of a given type of residue. In one embodiment, the immunomodulatory agent is attached to one or more of the constant domains and/or stabilization domains. In other embodiments, the agent is attached to the specificity domain. One of skill in the art will recognized that site of attachment of the agent will depend on the desired ligand and will be such as to not disrupt the interaction of the surrogate antibody with the target ligand.

Various immunomodulatory agents find use in the present invention. The immunomodulatory agent incorporated into the bi-functional surrogate antibody structure is selected depending on the ligand of interest and/or the type of immune response desired at the site of the ligand in the subject receiving the bi-functional antibody.

Immunomodulatory agents include, but are not limited to, polypeptides (such as, immunoglobulin heavy chains, cytokines, cytokine antagonist, polypeptides of the complement system, and heat shock proteins (i.e., the mycobacterial heat shock protein HSP65 (Silva et al. (1996) Infect. Immun. 64:2400-2407)). Additional immunomodulatory agents include nonproteinaceous polymers (see, U.S. Pat. No. 6,468,532), CpG motifs and active variants thereof, saponins and derivatives thereof (such as triterpenoid glycosides, QS-21, Kim et al. (2000) Vaccine 19:530-7), bacterial toxins and their variants and derivatives, lipopolysaccharide derivatives, Muramyl Dipeptide (MDP) and derivatives thereof (Ellouz et al. (1974) Biochem. Bioophys. Res. Commun. 59:1317-25, Azuma et al. (1992) Int. J. Immunopharmacol. 14:487-96, and O'Reilly et al. (1992) Clin. Infect. Dis. 14: 1100-9), hormones (i.e., 1α,25-dihydroxy vitamin D3 or Dehydroepiandrosterone (DHEA) (Daynes et al. (1996) Infect. Immun. 64:1100-9, Enioutina et al. (1999) Vaccine 1 7:3050-64, Van der Stede et al. (2001) Vaccine 19:1807-8, and Kriesel et al. (1999) Vaccine 17:1883-8), vitamins (Tengerdy et al. (1989) Ann. N. Y. Acad. Sci. 570:335-44 and Banic et al. (1982) Int. J. Vitam. Nurt. Res. Suppl 23:49-52) and imidazoquinolines (such as R-837, R-848) (Wagner et al. (1999) Cell Immunol 191:10-9 and Bernstein et al. (1993) J. Infect. Dis. 167:731-5). Immunolomodulaory agents further include adhesion molecules and active variants and fragments thereof including, but not limited to, selectins, cadherins, integrins, mucin-like vascular addressins, integrins, and immunoglobulin super family (CD2, CD54, CD102, lymphocyte antigen presenting cells like LFA3 and CD106). See, for example, U.S. Pat. No. 6,406,870, U.S. Pat. No., 6,123,915, U.S. Pat. No. 6,482,840, and U.S. Pat. No. 5,714,147, all of which are herein incorporated by reference. Additional exemplary agents are described in further detail below.

In other embodiments, the immunomodulatory agent can comprise any compound that that is foreign to the host (e.g., xenobiotic proteins such as BSA, mouse Ig, etc) that upon administration would potentiate a directed anti-surrogate antibody response at the site of the target ligand. A focused inflammatory response comprising, for example, complement activation, opsonization induced phatocytosis, ect., could ensue.

When the immunomodulatory agent is a polypeptide, the polypeptide could comprise biologically active variants and fragments of the sequences. Suitable biologically active variants can be fragments, analogues, and derivatives of the immunomodulatory agent (i.e, constant domains of immunoglobulins (IgG1, IgG2, IgG3, IgG4, IgA1, IgA2, IgM, IgD, IgE); cytokines; chemokines cytokine antagonists; HSP, etc.). By “fragment” is intended a protein consisting of only a part of the polypeptide sequence that retains biological activity (i.e., modulates the immune response). The fragment can be a C-terminal deletion or N-terminal deletion of the polypeptide. By “variant” of polypeptide capable of modulating an immune response (i.e., a constant domain of an immunoglobulin (IgG1, IgG2, IgG3, IgG4, IgA1, IgA2, IgM, IgD, IgE); cytokines; chemokines; cytokine antagonists; HSP, etc.) is intended an analogue of either the full length polypeptide capable of modulating an immune response, or a fragment thereof, that includes a native sequence and structure having one or more amino acid substitutions, insertions, or deletions. By “derivative” of a polypeptide capable of modulating an immune response (i.e., constant domain of immunoglobulins (IgG1, IgG2, IgG3, IgG4, IgD, IgA1, IgA2, IgE, IgM etc.) cytokines; chemokines; cytokine antagonist; and HSP, etc) is intended any suitable modification of the native polypeptide or fragments thereof, or their respective variants, such as glycosylation, phosphorylation, or other addition of foreign moieties, so long as the activity is retained.

Preferably, naturally or non-naturally occurring variants of a polypeptide capable of modulating an immune response (i.e., constant domain of an immunoglobulin, cytokines, chemokines, cytokine antagonist, heat shock proteins, etc.) have amino acid sequences that are at least 70%, preferably 80%, more preferably, 85%, 90%, 91%, 92%, 93%, 94% or 95% identical to the amino acid sequence to the reference molecule, for example, the Fc domain of an immunoglobulin (i.e., IgG1, IgG2, IgG3, and IgG4). More preferably, the molecules are 96%, 97%, 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ by as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

As outlined above, the art provides substantial guidance regarding the preparation and use of such variants. A fragment of a polypeptide capable of modulating an immune response will generally include at least about 10 contiguous amino acid residues of the full-length molecule, about 15-25 contiguous amino acid residues of the full-length domain, or about 20-50 or more contiguous amino acid residues of full-length constant domain.

When the agent(s) capable of modulating the immune response are non-proteinaceous molecule(s), the agent(s) can comprise active derivatives. By “derivative” of an agent capable of modulating an immune response (i.e., hormones (1α,25-dihydroxy vitamin D3 or Dehydroepiandrosterone (DHEA); vitamins; imidazoquinolines (such as R-837, R-848, etc.) is intended any suitable modification of the native agent, such as glycosylation, phosphorylation, other addition of foreign moieties, or alteration of native structure, so long as the desired activity is retained (i.e., modulation of an immune response).

It is further recognized that surrogate antibodies may be made to be less immunogenic by isolating a surrogate antibody composed exclusively of nucleic acid sequences having the minimum sequence length needed to maintain assembly for the intended application and by humanizing the sequence and/or decreasing the size of the peptide required to form the stabilization domain. In addition, the immunomodulatory agents attached to the bi-functional surrogate antibody may also be “humanized” forms of non-human polypeptides. In these embodiments, the amino acids from the donor polypeptide are replaced by corresponding human residues. Furthermore, a humanized polypeptide may comprise residues that are not found in the human sequence or in the donor antibody. These modifications are made to further refine the performance of the polypeptide. For further details, see Jones et al. (1986) Nature 321:522-525; Riechmann et al. (1988) Nature 332:323-329; and Presta et al. (1992) Curr. Op. Struct. Biol. 2:593-596.

i. Immunoglobulin Constant Chains

In one embodiment, the immunomodulatory agent capable of modulating an immune response comprises an amino acid sequence comprising a constant region from an immunoglobulin or an active variant or active fragment thereof.

By “constant region” of an immunoglobulin is intended the amino acid region of an immunoglobulin protein that confers the isotype-specific properties or the effector functions of the immunoglobulin. The constant region can comprise the constant domain of the light chain and the constant domains of the heavy chain. The constant domains are not involved directly in binding an antibody to a ligand, but exhibit various effector functions. Depending on the amino acid sequence of the heavy chain constant regions, immunoglobulins can be assigned to different classes. There are five major classes of immunoglobulins: IgA, IgD, IgE, IgG and IgM, and several of these may be further divided into subclasses (isotypes), e.g. IgG1, IgG2, IgG3, IgG4, IgA1, IgA2. The heavy chain constant regions that correspond to the different classes of immunoglobulins are called α, β, ε, γ, and μ, respectively. Any constant region of any immunoglobulin or an active variant or fragment thereof can be used as an immunomodulatory agent in the present invention. The amino acid sequences of the constant heavy immunoglobulin chain and the constant light immunoglobulin chains are set forth in Kabet et al. (1991) Sequences of Proteins ofImmunological Interest, 5^{th} Ed. Public Health Service, National Institute of Health, Bethesda, Md, the entire contents of which is herein incorporated by reference.

Active variants and fragments of these immunoglobulin constant chains are also known in the art and find use as immunomodulatory agents. An active variant or fragment of an immunoglobulin heavy chain will retain the ability to modulate the immune response, particularly the ability to modulate immune effector function. The effector functions mediated by the antibody constant regions include functions that operate after binding of the antibody to the antigen (i.e., by influencing the complement cascade, which can result in phagocytosis or complement dependent cytotoxicity, or Fc receptor (FcR) bearing cells). The constant region can also impart functions that operate independently of antigen binding (i.e., by conferring persistence in the circulation and the ability to be transferred across cellular barriers by transcytosis). See, Ward et al. (1995) Therapeutic Immunology 2:77-94.

Thus, an active fragment of a constant region of an immunoglobulin can comprise, for example, the heavy chain CH1 region, the heavy chain CH2 region, the heavy chain hinge region, the CH3 region, the CH4 region, the Kappa light chain, or any combination thereof, or alternatively the active fragment of the immunoglobulin constant region can comprise an Fc region. By “Fc region” is intended the C-terminal immunoglobulin that is produced upon digestion of the native antibody upon papain digestion (Deisenhofer et al. (1981) Biochemistry 20:2361-2370).

Thus, the constant domains of the immunoglobulin or the active fragments and variants thereof, when attached to the surrogate antibody of the invention can modulate the immune response in a variety of ways including modulation of opsonization, complement fixation, antigen clearance, ADCC, or cytotoxicity.

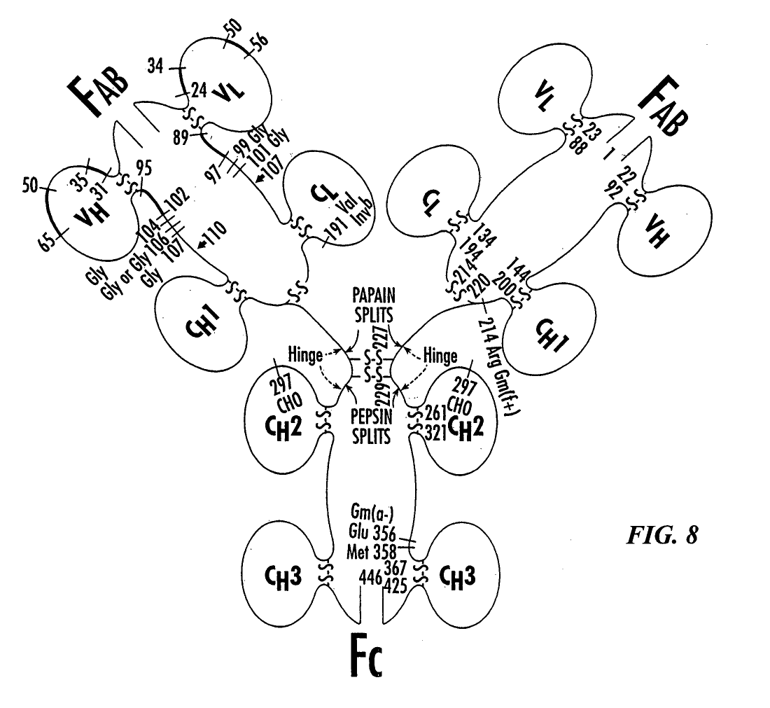

In one embodiment, the immunomodulatory agent is an IgG. In other embodiments, the immunomodulatory agent comprise the constant region of the IgG (i.e., IgG1, IgG2, IgG3, IgG4), and in other embodiments, the immunomodulatory agent comprises an active fragment or variant of the IgG constant regions (i.e., the heavy chain CH1 region, the heavy chain CH2 region, the heavy chain hinge region, the CH3 region, the CH4 region, the Kappa light chain, any combination thereof, or the Fc region). The amino acid sequence for these IgG domains is set forth in Kabet et al. (1991) Sequences of Proteins of Immunological Interest, 5^{th} Ed. Public Health Services, National Institute of Health, Bethesda, Md., volume 1: 661-723. Each of these pages is expressing incorporated herein by reference. A schematic diagram of an IgG molecule is set forth in FIG. 8.

The specific influence that the immunoglobulin constant regions or their active fragments or variants have on immune effector function is known and thus, one can design an immunoglobulin constant chain or a variant or fragment thereof that produces the desired modulation in the immune response. For example, though mediated by different cellular mechanisms, ADCC and phagocytosis have in common the initial binding of cell-bound mAbs, through their Fc region to the FcγR (i.e., FcγRI, FcγRII, and FcγIII). This interaction is followed by destruction of the target by the immune system cells. The interaction of the various IgG constant chains and active variant and fragments thereof with the various FcγR receptor types are know. Thus, a bi-functional surrogate antibody having an IgG constant domain or active fragment or variant thereof capable of binding the desired FcγR receptor will modulate an immune response (i.e., modulate the release of inflammatory mediators, endocytosis of immune complexes, modulates ADDC, acts as an cross-linking agent to FcγR-bearing cells, and an increase in immune system cell activation).

In one embodiment, the Fc region of the IgG immunoglobulin is used. In other embodiments, active variants and fragments of the IgG constant regions are used. Fc domains of the 4 IgG subclasses have different binding affinity to the various FcγR members. Such interactions are known in the art. See, for example, Gessner et al. (1998) Ann. Hematol 76:231-248, Warmerdam et al. (1991) J. Immunol. 147:1338-1343, de Haas et al. (1996) J. Immunol. 156:2948-2955, Koene et al. (1997) Blood 90: 1109-1114, Wu et al. (1997) J. Clin. Invest. 100:1059-1070, Kabat et al. (1991) Sequences of Proteins of Immunological Interest. 5^{th} Ed. Public Health Services, National Institutes of Health, Lund et al. (1995) FASEB J. 9: 115-119, and Morgan et al. (1995) Immunology 86:319-324, Michaelsen et al. (1992) Mol. Immunol. 29:319-326 and Shields et al. (2001) J. Biol. Chem. 267: 6591-6604, each of which is herein incorporated by reference. These references discuss the constant region of the IgG subclasses the mediate FcγR interaction. See, also U.S. Pat. No. 6,194,551 that discusses variants of immunoglobulins having this desired activity. The desired Fc domain for the desired immune modulation could therefore be designed.

Analogues of the IgG constant regions that interact with the FcγR are also known. For example, the sequences can comprise carbohydrate optimizations. For instance, the carbohydrate attached to Asn297 of the Fc domain influences interaction of IgG to FcγR and reduces ADCC activity. Thus, when an increase in effector function is desired, aglycosyl polypeptide could be used, or alternatively, the amino acid position at Asn297 can be altered to another amino acid. See, for example, Hobbs et al (1992) Mol. Immunol. 29:949-956. Additional, analogues may include attachment of various oliogsaccharides including galactose, galactose-sialic acid, mannose, fucose, and N-acetylglucosamine. For a review of additional active variants, see Presta et al. (2002) Current Pharmaceutical Biotechnology 3:237-256.

Another IgG-dependant effector system utilizes complement activation. Instead of immune system cells (as in ADCC and phagocytosis), the complement system is a series of soluble blood proteins which cascade to form a complex which kill cells either through a classical pathway (clq binding to IgG bound to cells) or through an alternative pathways utilizing initial binding of other molecules. Clq is a complement protein that must bind to multiple IgG attached to the cell surface in order to initiate the cascade.

The interaction of the various IgG constant regions with the Clq complement protein has been characterized. Thus, a bi-functional surrogate antibody having an immunoglobulin constant region or active fragment or variant thereof capable of activating the complement can be designed. The interaction of the bi-functional surrogate antibody comprising an immunoglobulin constant region or a variant or fragment thereof that is capable of interacting with C1q will posses the ability to modulate the immune response by modulating the complement cascade.

The IgG epitope for C1q interaction has been studied. Studies suggest Asp270, Lys322, Pro329, and Pro331 comprise the C1q-binding epitope. See, for example, Tao et al. (1993) J. Exp. Med. 1 78:661-667, Idusogie et al. (2000) J. Immunol. 164:4178-4184, and Thommesen et al. (2000) Mol. Immunol37:995-100⁴, each of which is herein incorporated by reference.

Variants and analogs of IgG constant chains that modulate the immune response via an interaction with C1q are also known. Studies on the effect of terminal sialic acid and terminal galactose also modulate complement activation. See, for example, Wright et al. (1998) J. Immunol. 160: 3393-3402, Jassal et al. (2001) Biochem. Biophys. Res. Commun. 286:243-249, Gottleib et al. (2002) J. Am. Acad. Dermatol. 43:595-604, all of which are incorporated by reference. In addition, amino acid residues in IgG1 have been identified which when modified increase complement activation. See, for example, Idusogie et al. (2001) J. Immunol. 166:2571-2575. See, also U.S. Pat. No. 6,194,551 that discusses variants of immunoglobulins having the desired activity.

Another effector function of IgG involves its half-life or clearance rate. Human IgG has a relatively long half-life. Thus, a bi-functional surrogate antibody having a constant domain of an immunoglobulin or an active variant or an active fragment thereof, will modulate an immune response by increasing the half-life of the bi-functional surrogate antibody. This modification could reduce the dosage or frequency of administration without affecting efficacy of the bi-functional surrogate antibody.

The half-life of immunoglobulins is influence by the interaction with FcRn. The epitope for IgG interaction with FcRn has been mapped (Kim et al. (1994) Eur. J Immunol. 24:542-548, Kim et al. (1994) Eur. J. Immunol. 24: 2429-2434, Kim et al. (1999) J. Immunol. 29:2819-2825, Medesan et al. (1997) J. Immunol. 158:2211-2217, and Weiner et al. (1995) Cancer Res. 55:4586-4593)) and it has been shown that alterations of specific amino acids in murine IgG that improve binding to murine FcRn also result in increased half-life in mice (Ghetie et al. (1997) Nature Biotechnol. 15:637-640). Thus, a number of variants of IgG could be generated which when attached to the bi-functional surrogate antibody of the instant invention will produce a half-life that is desirable for the intended application.

A constant region of IgA can also elicit immune effector function. For example, regions of the IgA constant chain that interact with FcαR1 are capable of modulating the immune response, including ADCC, neurtophil respiratory burst, and phagocytosis. See, for example, Morton et al. (1996) Crit. Rev. Immunol. 16: 423-440, Van Egmond et al. (2000) Nat. Med. 6:680-685, Van Egmond et al. (1999) Blood 93:4387-4394, Van Egmond et al. (1999) Immunol. Lett 68:83-87, U.S. Pat. No. 6,063, all of which are herein incorporated by reference. Active variants and fragments of IgA are known. See, for example, Mattu et al. (1998) J. Biol. Chem. 273:2260-2272, Rifai et al. (2000) J. Exp. Med. 191:2171-2181.

Variants of the immunoglobulins of the invention may further comprise humanized polypeptides. “Humanized” forms of non-human (e.g., murine) antibodies are chimeric antibodies that contain minimal sequence derived from non-human immunoglobulins. In these embodiments, the amino acids from the donor immunoglobulin are replaced by corresponding human residues. Furthermore, a humanized immunoglobulin may comprise residues that are not found in the human-antibody or in the donor antibody. These modifications are made to further refine antibody performance of the immunoglobulin domain. For further details, see Jones et al. (1986) Nature 321:522-525; Riechmann et al. (1988) Nature 332:323-329; and Presta et al. (1992) Curr. Op. Struct. Biol. 2:593-596.

In yet another embodiment, the immunoglobulin constant chain or active variant or fragment thereof attached to the bi-functional surrogate antibody acts as a transporting agent. By “transporting agent” is intended any molecule that is capable of undergoing transepithelia transport via transcytosis. Both IgA and IgM are secreted at the mucosal surface and can therefore act as transporting agents. As discussed above, two isoforms of IgA occur in humans, IgA1 and IgA2. Diameric IgA comprises two IgA molecules connected by a disulfide bond to a cysteine-rich polypeptide called the J-chain. The transfer of the dimeric IgA into mucosa is mediated by the polymeric immunoglobulin receptor (pIgR). This receptor can bind dimeric IgA at the basolateral surface of mucosal epithelial cells and the IgA/pIgR complex is then transcytosed to the apical cell surface. The diametric IgA/J chain/pIgR complex is released and thereby produces a secretory defense system at mucosal surfaces against pathogenic microorganism. Variants and fragments of IgA that have the transport activity are known. See, U.S. Pat. No. 6,063,905, herein incorporated by reference. See also, for example, Kerr et al. (1990) Biochem J. 271: 285-296, Morton et al. (1996) Crit. Rev. Immunol. 16:423-440, and, Chintalacharuvu et al. (1999) Immunotechnology 4:165-174, U.S. Pat. No. 5,928,895, U.S. Pat. No. 6,045,774. In one embodiment, the transport agent comprises the secretory domain of IgA or IgM. See, for example, U.S. Pat. No. 6,063,905, herein incorporated by reference.

Assays for the transport of the bi-functional surrogate antibody having a transporting agent attached thereto are known in the art. Such assays include assaying for binding activity and specificity to pIgR (Bakos et al. (1991) J. Immunol. 147:3419-3426). In addition, the field of Fc structure/function has been developed using various in vitro expression systems that allow the production of active fragments and variant of immunoglobulins. These systems have facilitated the elucidation of complement and/or Fc receptor binding sites on IgM, IgG, and IgE (Burton et al. (1992) Adv. Immunol. 51:1-84). Similar assay systems have been used to study IgA and IgM and active variant and fragments that retain pIgR binding activity and thus allow for mucosal transport. In addition, in vitro transcytosis assays are known. Briefly, the MDCK (Madin-Darby canine kidney) cell line, transfected with pIgR, has been used to assay for transcytosis. This is a polarized cell line, capable of forming monolayers with tight junctions, which when grown on a semipermeable support will transport IgA, IgM or active variants and active fragments thereof having transport activity from the lower (basolateral) to the upper (apical) chamber of a tissue culture well.

Accordingly, in another embodiment, the bi-functional surrogate antibodies can comprise one or more of the same or different transporting agent(s) attached thereto. The molecule having the transporting agents can further comprise one or more immunomodulatory agents or other functional moiety as discussed elsewhere herein.

ii. Bispecific Antibodies

In another embodiment, the bi-functional surrogate antibody molecule of the invention is designed to be a bispecific antibody. Bispecific antibodies are antibodies that comprise two specificities (i.e., they bind two different epitopes on two different antigens). In this embodiment, the immunomodulatory agent comprises a specificity domain that is capable of interacting with an immune response regulator. As used herein, an “immune response regulator” is any molecule which when brought to the site of the ligand/surrogate antibody interaction is capable of producing a modulation in the immune response.

For example, in one embodiment, the surrogate antibody comprises a first specificity domain that interacts with a target ligand and a second specificity domain that interacts with an immune response regulator, such as an FcyR. Thus, the interaction with FcγR will recruit immune effector cells to destroy the target antigen. See, for example, Da Costa et al. (2000) Cancer Chemother. Pharmacol. 46:S33-S36, McCall et al. (1999) Mol. Immunol 36:433446, Akewanlop etal. (2001) Cancer Res. 61:4061-4065, Sundarapandiyan et al. (2001) J. Immunol. Meth 248:113-123, and, Stockmeyer et al. (2001) J. Immunol. Meth. 248:103- 111, all of which are herein incorporated by reference. Other immune response regulators include, but are not limited to, alpha 1 anti-trypsin and a major histocompatibility complex (i.e., histocompatibility antigens associated with tumor specific antigens or viral associated antigens).

iii. Cytokines

Cytokines are immunomodulatory molecules that effect a abroad range of immune cell types. As used herein, the term “cytokine” refers to a member of the class of proteins that are produced by cells of the immune system and that regulate or modulate an immune response. Such regulation can occur within the humoral or the cell mediated immune response and includes modulation of the effector function of T cells, B cells, NK cells macrophages, antigen presenting cells or other immune system cells. Attachment of a cytokine to the surrogate antibody of the invention will allow for the targeted delivery of the cytokine to the target ligand (i.e., a cancer cell, a bacteria, a virus) and thus the targeted delivery of the cytokine at the desired site will reduce the toxicity of cytokines frequently observed upon systemic administration.

As used herein, the term cytokine encompasses those cytokines secreted by lymphocytes and other cell types (designated lymphokines) as well as cytokines secreted by monocytes and macrophages and other cell types (designated monokines). The term cytokine includes the interleukins, such as IL-2 (Harvill et al. (1995) Immunotechnology 1: 95-105 and Shu et al. (1995) Immunotechnology 1: 231-241), IL-3, IL-4, and IL-12 (Lode et al. (1998) Proc. Natl. Acad. Sci. USA 95:2475-2450 and Peng et al. (1999) J. Immunol. 163:250-258, Kenney et al. (1999) J. Immunol 163:4481-8 and Buchanan et al. (1998) J. Immunol 161:5525-33), which are molecules secreted by leukocytes that primarily affect the growth and differentiation of hematopoietic and immune-system cells.

The term cytokine also includes hematopoietic growth factors and, such as, colony stimulating factors such as colony stimulating factor-i (Nobiron et al. (2001) Vaccine 19:4226-35 and Dela et al. (2000) J. Immunol. 165:5112-5121), granulocyte colony stimulating factor and granulocyte macrophage colony stimulating factor (U.S. Pat. No. 6,482,407). In addition, the term cytokine encompasses chemokines, which are low-molecular weight molecules that mediate the chemotaxis of various leukocytes and can regulate leukocyte integrin expression or adhesion. Exemplary chemokines include interleukin-8 (Holzer et al. (1999) Cytokine 8:214-221), dendritic cell chemokine 1 (DC-CK1) and lymphotactin, which is a chemokine important for recruitment of T cells and for mucosal immunity, as well as other members of the C—C and C—X—C chemokine subfamilies. The CXC family members are characterized as having two cysteine residues separated by another amino acid and function to promote migration of neurophiles and examples include IL8, IP10, SDF1. CC family members promote migration of monocytes or other cell types and examples include macrophage chemoattractant protein or MCP1, MIPα and β, RANTES, Eotaxin, Lymphotactin (attracts T-cell precursor in the thymus). Members of the CXXXXC family include fractalkine which attracts monocytes and T-cells. See, for example, Miller et al. (1992) Crit. Rev. Immunol. 12:17-46 (1992); Hedrick et al. (1997) J. Immunol. 158:1533-1540; and Boismenu et al. (1996) J. Immunol. 157:985-992, each of which are incorporated herein by reference.

The term cytokine, as used herein, also encompasses cytokines produced by the T helper 1 (T_{H1}) and T helper 2 (T_{H2}) subsets. Cytokines of the T_{H1} subset are produced by T_{H1} cells and include IL-2, IL-12, IFN-alpha and TNF-beta. Cytokines of the T_{H1} subset are responsible for classical cell-mediated functions such as activation of cytotoxic T lymphocytes and macrophages and delayed-type hypersensitivity. Cytokines of the T_{H1} subset are particularly useful in stimulating an immune response to tumor cells, infected cells and intracellular pathogens.

Cytokines of the T_{H2} subset are produced by T_{H2} cells and include the cytokines IL-4, IL-5, IL-6 and IL-10 (Kim et al. (1999) J. Med. Primatol. 28:214-23 and Suh et al. (1999) J. Interferon Cytokine Research 19:77-84). Cytokines of the T_{H2} subset function effectively as helpers for B-cell activation and are particularly useful in stimulating an immune response against free-living bacteria and helminthic parasites. Cytokines of the T_{H2} subset also can mediate allergic reactions. Thus, any cytokine can be attached to the surrogate antibody. See also U.S. Pat. No. 6,399,068. Additional cytokines of interest include, lymphotoxin and TGF-β.

Active fragments and variants of cytokines are also useful in the invention. Active cytokine fragments and variants are known in the art and include, for example, a nine-amino acid peptide from IL-1β that retains the immunostimulatory activity of the full-length IL-1β cytokine. See, Hakim et al. (1996) J. Immunol. 157:5503-5511, which is incorporated herein by reference. In addition, a variety of well known in vitro and in vivo assays for cytokine activity, such as the bone marrow proliferation assay described in U.S. Pat. No. 6,482,407, are useful in testing a cytokine fragment for activity. See, also Thomson (1994) The Cytokine Handbook (Second Edition) London: Harcourt Brace & Company. Both of these references are herein incorporated by reference.

A cytokine antagonist also can be an immunomodulatory molecule useful in the invention. Such cytokine antagonists can be naturally occurring or non-naturally occurring and include, for example, antagonists of GM-CSF, G-CSF, IFN-γ, IFN-α, TNF-α, TNF-β, IL-1, IL-2, IL-3, IL-4, IL-6, IL-7, IL-10, IL-12, lymphotactin and DC-CK1. Cytokine antagonists include cytokine deletion and point mutants, cytokine derived peptides, and soluble, dominant negative portions of cytokine receptors. Naturally occurring antagonists of IL-1, for example, can be used as an immunomodulatory agent of the invention to inhibit the pathophysiological activities of IL-1. Such IL-1 antagonists include IL-1Ra, which is a polypeptide that binds to IL-1 receptor I with an affinity roughly equivalent to that of IL-1α or IL-1β but that does not activate the receptor (Fischer et al. (1991) Am. J. Physiol. 26]:R442-R449; Dinarello et al. (1991) Immunol. Today 12:404-410, each of which are incorporated herein by reference). IL-1 antagonists also include IL-1β derived peptides and IL-1 muteins (Palaszynski et al. (1987) Biochem. Biophys. Res. Commun. 147:204-209, which is incorporated herein by reference). Cytokine antagonists useful in the invention also include, for example, antagonists of TNF-α (Ashkenazi et al. (1991) Proc. Natl. Acad. Sci. USA 88:10535-10539; and, Mire-Sluis et al. (1993) Trends in Biotech. 11:74-77, each of which are incorporated herein by reference).

iv. Immunomodulatory Nucleic Acid Motifs

In another embodiment of the invention, the bi-surrogate antibody has attached thereto an immunomodulatory nucleic acid motif. A “CpG motif” as used herein comprises an unmethylated cytosine, guanine dinucleotide sequence (i.e., CpG motif which comprises a cytosine followed by a guanine linked by a phosphate bond) that is capable of modulating an immune response.

In one embodiment, the immunomodulatory nucleic acid motif comprises an immunostimulatory nucleic acid motif. As used herein, an “immunostimulatory nucleic acid motif” this is capable of stimulating an immune response and comprises an unmethylated cytosine, guanine dinucleotide sequence (i.e., CpG motif which comprises a cytosine followed by a guanine linked by a phosphate bond). Such a stimulation can comprise a mitogenic effect on or an increase in cytokine expression by vertebrate lymphocytes. Stimulatory CpG motifs also, for example, increase natural killer cell lytic activity, modulate antibody dependant cellular cytotoxicity (ADCC), and/or activate B-cells dendritic cells and T-cells. Thus, a bi-functional specific antibody having an immunostimulatory nucleic acid motif finds use in the present invention.

Various immunostimulatory CpG motifs are know. See, for example, U.S. Pat. No. 6,339,068, U.S. Pat. No. 6,476,000, Klinman et al. (2002) Microbes and Infection 4:897-901, McKenzie et al. (2001) Immunological Research 24:225-244, and Carpentier et al. (2003) Frontiers in Bioscience 8:115-127, all of which are herein incorporated by reference. Typical immunostimulatory CpG motif will comprise 5′ N₁CGN₂ 3′, (SEQ ID NO:5) wherein at least one nucleotide separates consecutive CpGs motifs and N₁ is adenine, guanine, or thymine/uridine and N₂ is cytosine, thymine/uridine, or adenine. Exemplary immunostimulatory CpG oligonucleotide motifs include GACGTT (SEQ ID NO:6), AGCGTT (SEQ ID NO:7), AACGCT (SEQ ID NO:8), GTCGTT (SEQ ID NO:9), and AACGAT (SEQ ID NO:10). Another immunostimulatory nucleic acid motifs include TCAACGTT (SEQ ID NO:11). Further exemplary oligonucleotides of the invention contain GTCG(T/C)T (SEQ ID NO:12), TGACGTT (SEQ ID NO:13), TGTCG(T/C)T (SEQ ID NO: 14), TCCATGTCGTTCCTGTCGTT (SEQ ID NO: 15), TCCTGACGTTCCTGACGTT (SEQ ID NO:16) and TCGTCGTTTTGTCGTTTTGTCGTT (SEQ ID NO:17).

In other embodiments, an immunosuppressive nucleic acid motif can be incorporated into the surrogate antibody molecule. Such motifs include CpG motifs containing direct repeats of CpG dinucleotides, CCG trinucleotides, CGG trinucleotides, CCGG tetranucleotides, CGCG tetranucleotides or a combination of any of these motifs. See, also Carpentier et al. (2003) Frontiers in Bioscience 8:115-127.

The exact immunomodulatory CpG motif to be added will depend on the ultimate purpose of the bi-functional surrogate antibody. For example, if the bi-functional surrogate antibody is used to treat an infection, then motifs that preferentially induce cell-mediated immunity and/or a particular cytokine profile, will be introduced into the bi-functional surrogate antibody. Method for assaying for the immunostimulatory effect of CpG sequences are known. For example, there is a strong correlation between certain in vitro immunostimulatory effects and in vivo effects of specific CpG motifs. For example, the strength of the humoral response correlates very well with the in vitro induction of TNF-alpha, IL-6, IL-12, and B-cell proliferation. The strength of the cytotoxic T-cell response correlates well with the in vitro induction of IFN-gamma. See, for example, U.S. Pat. No. 6,339,068, Krieg et al. (2002) Annu. Rev. Immunol 20:709-760, Krieg et al. (1995) Nature 374:546-549, Yi et al. (1996) J. Immunol 157:5394-5402, Stacey et al. (1996) J. Immunol 157:2116-2122, Cho et al. (2000) Nat. Biotechnol 18:509-514, Iho et aL (1999) J. Immunol. 163:3642-3652, all of which are herein incorporated by reference.

Active variants, fragments and analogues of these various CpG motifs can also be used as immunomodulatory agents in the present invention. The active variants and fragments of the CpG motifs will retain the ability to modulate the immune response. As discussed above, various assays are known to determine if the CpG sequence retains the desired immunomodulatory activity. An active variant or analogue of a CpG sequence will maintain the immunomodulatory activity and comprise at least 70%, 75%, 80%, 85%, 90%,95%, 96%, 97%, 98%, 99% sequence identity to the reference CpG sequence. Methods for determining % identity for a nucleotide sequence are discussed elsewhere herein.

v. Lipopolysaccharide and Derivatives Thereof

LPS is a potent immunomodulator and inducer of cytokines, such as IL-1, IL-6, and TNF-alpha. Active derivatives of LPS are known. For example, derivatives of lipid A have been produce that retain the immunostimulatory activity of lipid A yet reduce the toxicity. Such active derivatives include monophosphoryl lipid A (MPL) that has been shown to enhance both humor and cellular immune response. See, for example, Kiener et al. (1988) J. Immunol 141:870-4 and Childers et al. (2000) Infect. Immun. 68:5509-16.

F. Additional Functional Moieties

As discussed above, the residues (i.e., nucleotides or amino acid residues) used to prepare the bi-functional surrogate antibodies (i.e., the specificity strand and the stabilization strand) can be naturally occurring or modified. Such modifications include alterations in the components of the specificity strand or the stabilization strand that results in the attachment of a “functional moiety” with the bi-functional surrogate antibody. As discussed above, attachment is any association (including a covalent, ionic, hydrophobic ect.) that allows for the formation of a stable interaction with the surrogate antibody under the conditions of the intended application.

In any of the various methods and compositions described herein, various functional moieties (1, 2, 3, 4, 5 or more) can be associated with one or more strands that form the bi-functional antibodies, in one or more positions on the strands. The functional moiety can be at either or both of the terminal ends of either the stabilization strand or the specificity strand, added to individual residues anywhere in either strand, attached to all or a portion of the residues (i.e., pyrimidines or purines), attached to all or a portions of a given type of residue (i.e., A, G, C, T/U), and or attached to any region of a residue (i.e., a sugar, a phosphate, a nitrogenous base). In one embodiment, the functional moiety is attached to one or more of the constant domains and/or stabilization domains. In other embodiments, the functional moiety is associated with the specificity domain. One of skill in the art will recognize that the site of association of the functional moiety will depend on the desired functional moiety. In addition, the functional moiety(ies) chosen to incorporate into the bi-functional surrogate antibody structure can be selected depending on the conditions in which the bi-functional surrogate antibody will be contacted with its ligand or potential ligand.

Examples of these modifications in the bi-functional surrogate antibody molecule include nucleotides that have been modified with amines, diols, thiols, phophorothioate, glycols, fluorine, hydroxl, fluorescent compounds (e.g. FITC), avidin, biotin, aromatic compounds, alkanes, and halogens. Such modifications can further include, but are not limited to, modifications at cytosine exocyclic amines, substitution of 5-bromo-uracil (Golden et al. (2000) J. of Biotechnology 81:167-178), backbone modifications, methylations, unusual base-pairing combinations and the like. See, for a review, Jayasena et al. (1999) Clinical Chemistry 45:1628-1650.

Those of skill in the art are aware of numerous modifications to nucleotides and to phosphate linkages between adjacent nucleotides that render them more stable to exonucleases and endonucleases (Uhlmann et al. (1990) Chem Rev. 90:543-98 and Agraul et al. (1996) Trends Biotechnology 14:147-9 and Usman et al. (2000) The Journal of Clinical Investigations 106:1197-1202). Such functional moieties include, for example, modifications at the 2′ position of the sugars (Hobbs et al. (1973) Biochemistry 12:5138-45 and Pieken et al. (1991) Science 253:314-7). For instance, the modified nucleotide could be substituted with amino and fluoro functional groups at the 2′ position. In addition, further functional moieties of interest include, 2′-O-methyl purine nucleotides and phosphorothioate modified nucleotides (Green et al. (1995) Chem. Biol. 2:683-695; Vester et al. (2002) J. Am. Chem. Soc. 124:13682-13683; Rhodes et al. (2000) J. Biol. Chem. 37:28555-28561; and, Seyler et al. (1996) Biol. Chem. 377:67-70). Accordingly, in another embodiment, the bi-functional surrogate antibody molecules comprise functional moieties comprising modified nucleotides that stabilize the molecule in the presence of serum nucleases.

Other functional moieties of interest include chemical modifications to one or more nucleotides in the specificity domain of the specificity strand, wherein the modified nucleotide introduces hydrophobic binding capabilities into the specificity domain. In certain embodiments, this chemical modification occurs at the 2′ position of the nucleotide sugar or phosphate molecule. Such modifications are known in the art and include for example, non-polar, non-hydrogen binding shape mimics such as 6-methyl purine and 2,4-difluorotolune (Schweizer et al. (1995) J Am Chem Soc 117:1863-72 and Guckian et al. (1998) Nat Struct Biol 5:950-9, both of which are herein incorporated by reference). Additional modifications include imizadole, phenyl, proline, and isoleucyl.

In other embodiments, it is desirable to preferentially amplify the specificity strand of the bi-functional surrogate antibody molecule. By “preferentially amplify” is intended that the specificity strand of the bi-functional surrogate antibody molecule is amplified during the amplification step at an elevated frequency as compared to the amplification level of the corresponding stabilization strand. As such, an additional functional moiety of interest comprises a modification that allows for the preferential amplification of the specificity strand of the bi-functional surrogate antibody molecule. While methods of amplifying the bi-functional surrogate antibodies are discussed in further detail elsewhere herein, the type of modification that would allow this type of amplification are known in the art, and include, for example, a modification to at least one nucleotide on the stabilization strand that increases resistance to polymerase activity in a PCR reaction. Such modifications include any functional moiety that disrupts amplification including, for example, biotin.

Additional functional moieties of interest include, for example, a reporter molecule. As used herein a “reporter molecule” refers to a molecule that permits the detection of the bi-functional surrogate antibody that it is attached to. Accordingly, in another embodiment, the incorporation or attachment of a “reporter” molecule as a functional moiety permits detection of the surrogate antibody and the complexed ligand. Such reporter molecules include, for example, a polypeptide; radionucleotides (e.g. ³²P); fluorescent molecules ((Jhaveri et al. (2000) J. Am. Chem. Soc. 122:2469-2473), luminescent molecules, and chromophores (such as FITC, Fluorescein, TRITC, Methyl Umbiliferone, luminol, luciferin, and Texas Red (Sumedha et al. (1999) Clinical Chemistry 45:1628-1649 and Wilson et al. (1998) Clin Chemistry 44:86-91, and (2000) Nature Biotechnology 18:345-349)); enzymes (e.g. Horseradish Peroxidase, Alkaline Phosphatase, Urease, β-Galactosidase, Peroxidase, proteases, etc.), lanthanide series elements (e.g. Europium, Terbium, Yttrium), and microspheres (e.g. sub-micron polystyrene, dyed or undyed). Such reporter molecules allow for direct qualitative or quantitative detection or energy transfer reactions.

In yet other embodiments, the functional moiety is incorporated into the specificity strand to expand the genetic code. Such moieties include, for example, IsoG/IsoC pairs and 2,6-diaminopyrimide/xanthine base pairs (Piccirilli et al. (1990) Nature 343:537-9 and Tor et al. (1993) J Am Chem Soc 115:4461-7); methyliso C and (6-aminohexyl)isoG base pairs (Latham et al. (1994) Nucleic Acid Research 22:2817-22), benzoyl groups (Dewey et al. (1995) J Am Chem Soc 11 7:8474-5 and Eaton et al. (1997) Curr Opin Chem Biol 1:10-6) and amino acid side chains.

Other functional moieties of interest include a linking molecule (i.e., iodine or bromide for either photo or chemical crosslinking; a —SH for chemical crosslinking); a therapeutic agent (i.e., compounds used in the treatment of cancer, arthritis, septicemia, myocardial arrhythmia's and infarctions, viral and bacterial infections, autoimmune diseases and prion diseases); a chemical modification that alters biodistribution, pharmacokinetics and tissue penetration, or any combination thereof. Such modifications can be at the C-5 position of the pyrimidine residues.

Functional moieties incorporated into the bi-functional surrogate antibody (either in the stabilization strand or the specificity strand or both) may be multi-functional (i.e., the moiety could allow for labeling and affinity delivery, nuclease stabilization and/or produce the desired multi-therapeutic or toxicity effects. These various “functional moiety” modifications find use, for example, in aiding detection for applications such as fluorescence-activated cell sorting (Charlton et al. (1997) Biochemistry 36: 3018-3026 and Davis et al. (1996) Nucleic Acid Research 24:702-703), enzyme-linked oligonucleotide assays (Drolet et al. (1996) Nat. Biotech 14:1021-1025). In addition, conjugation with a technetium-99m chelation cage would enable in vivo imaging. See, for example, Hnatowich et al. (1998) Nucl. Med. 39:56-64.

Additional functional moieties of interest include the addition of polyethylene glycerol to decrease plasma clearance in vivo (Tucker et al. (1999) J. Chromatography 732:203-212 or the addition of a diacylglycerol lipid group (Willis et al. (1998) Bioconjugate Chem. 9:573-582). In addition, the functional moiety having anti-microbial activity (i.e., anti-bacterial, anti-viral, or anti-fungal) properties could be used with the surrogate antibody as an anti-bioterror agent to overwhelm native or modified pathogenic organisms and viruses.

In one embodiment, the functional moiety is digoxigenin. Detection of this functional moiety is achieved by incubation with anti-digoxigenin antibodies coupled directly to several different fluorochromes or enzymes or by indirect immunofluorescence. See, Ausubel et al. Current Protocols in Molecular Biology, John Wiley & Sons, Inc. and Celeda et al. (1992) Biotechniques 12:98-102, both of which are herein incorporated by reference. Additional molecules that can act as reporters include biotin and polyA tails.

In another embodiment, the functional moiety is an affinity tag that can be used to attach bi-functional surrogate antibodies to a solid support or to other molecules in solution. Thus, the isolation of the ligand-bound bi-functional surrogate antibody complexes can be facilitated through the use of affinity tags coupled to the surrogate antibody. As used herein, an affinity tag is any compound that can be associated with a surrogate antibody molecule and which can be used to separate compounds and/or can be used to attach compounds to the surrogate antibody. Preferably, an affinity tag is a compound that binds to or interacts with another compound, such as a binding molecule or an antibody. It is also preferred that such interactions between the affinity tag and the capturing component be a specific interaction. For example, when attaching surrogate antibody molecules to a column, microplate well, or tube containing immobilized streptavidin, surrogate antibody molecules prepared using biotinylated primers result in their binding to the streptavidin bound to the solid phase. Other affinity tags used in this manner can include a polyA sequence, protein A, receptors, antibody molecules, chelating agents, nucleotide sequences recognized by anti-sense sequences, cyclodextrin, and lectins. Additional affinity tags, described in the context of nucleic acid probes, have been described by Syvanen et al. (1986) Nucleic Acids Res. 14:5037. Preferred affinity tags include biotin, which can be incorporated into nucleic acid sequences (Langer et al. (1981) Proc. Natl. Acad Sci. USA 78:6633) and captured using streptavadin or biotin-specific antibodies. A preferred hapten for use as an affinity tag is digoxygenin (Kerkhof (1992) Anal. Biochem. 205:359-364). Many compounds for which a specific antibody is known or for which a specific antibody can be generated can be used as affinity tags. Antibodies useful as affinity tags can be obtained commercially or produced using well-established methods. See, for example, Johnston et al. (1987) Immunochemistry In Practice (Blackwell Scientific Publications, Oxford, England) 30-85.

Other affinity tags are anti-antibody antibodies. Such anti-antibody antibodies and their use are well known. For example, anti-antibody antibodies that are specific for antibodies of a certain class or isotype or sub-class (for example, IgG, IgM), or antibodies of a certain species (for example, anti-rabbit antibodies) are commonly used to detect or bind other groups of antibodies. Thus, one can have an antibody to the affinity tag and then this antibody:affinity tag:surrogate antibody complex can then be purified by binding to an antibody to the antibody portion of the complex.

Another affinity tag is one that can form selectable cleavable covalent bonds with other molecules of choice. For example, an affinity tag of this type is one that contains a sulfur atom. A nucleic acid molecule that is associated with this affinity tag can be purified by retention on a thiopropyl sepharose column. Extensive washing of the column removes unwanted molecules and reduction with β-mercaptoethanol, for example, allows the desired molecules to be collected after purification under relatively gentle conditions.

In addition, aptamers known to bind, for example, cellulose (Yang et al. (1998) Proc. Natl. Acad. Sci. 95: 5462-5467) or Sephadex (Srisawat et al. (2001) Nucleic Acid Research 29) have been identified. These aptamers could be attached to the surrogate antibody and used as a means to isolate or detect the surrogate antibody molecules.

Various methods for associating the functional moiety to the surrogate antibody structure are known in the art. For example, bioconjugation reactions that provide for the conjugation of polypeptides or various other compounds of interest to the surrogate antibody can be found, for example, in Aslam et al. (1999) Protein Coupling Techniques for Biomed Sciences, Macmillan Press; Solulink Bioconjugation systems at www.solulink.com; Sebestyen et al. (1998) Nature Biotechnology 16:80-85; Soukchareum et al. (1995) Bioconjugate chem. 6:43-54; Lemaitre et al. (1987) Proc. Natl Acad Sci USA 84:648-52 and Wong et al. (2000) Chemistry of Protein Conjugation and Cross-Linking, CRC, all of which are herein incorporated by reference.

Additional functional moieties include various agents that one desires to be directed to the location of the target ligand. The agent for delivery can be any molecule of interest, including, a therapeutic agent or a drug delivery vehicle. Such agents and their method of deliveries are disclosed elsewhere herein.

II. Pharmaceutical Compositions

The bi-functional surrogate antibody molecule of the invention may further comprise an inorganic or organic, solid or liquid, pharmaceutically acceptable carrier. The carrier may also contain preservatives, wetting agents, emulsifiers, solubilizing agents, stabilizing agents, buffers, solvents and salts. Compositions may be sterilized and exist as solids, particulates or powders, solutions, suspensions or emulsions.

The bi-functional surrogate antibody can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton Pa. (1980)). In order to form a pharmaceutically acceptable composition suitable for effective administration, such compositions will contain an effective amount of the bi-functional surrogate antibody molecule, either alone, or with a suitable amount of carrier vehicle.

The pharmaceutically acceptable carrier will vary depending on the method of administration and the intended method of use. The pharmaceutical carrier employed may be, for example, either a solid, liquid, or time release. Representative solid carriers are lactose, terra alba, sucrose, talc, gelatin, agar, pectin, acacia, magnesium stearate, stearic acid, microcrystalin cellulose, polymer hydrogels, and the like. Typical liquid carriers include syrup, peanut oil, olive oil, cyclodextrin, and the like emulsions. Those skilled in the art are familiar with appropriate carriers for each of the commonly utilized methods of administration. Furthermore, it is recognized that the total amount of bi-functional surrogate antibody administered will depend on both the pharmaceutical composition being administered (i.e., the carrier being used), the mode of administration, binding activity, and the desired effect (i.e., a modulation in the immune response). The amount of the bi-functional surrogate antibody administered will be sufficient to produce the desired modulation in the immune response.

Once the pharmaceutical composition has been formulated, it may be stored in sterile vials as a solution, suspension, gel, emulsion, solid, or dehydrated or lyophilized powder. Such formulations may be stored either in a ready to use form or requiring reconstitution immediately prior to administration.

The bi-functional surrogate antibodies also can be delivered locally to the appropriate cells, tissues or organ system by using a catheter or syringe. Other means of delivering bi-functional surrogate antibodies locally to cells include using infusion pumps (for example, from Alza Corporation, Palo Alto, Calif.) or incorporating the surrogate antibodies into polymeric implants (see, for example, Johnson eds. (1987) Drug Delivery Systems (Chichester, England: Ellis Horwood Ltd.), which can affect a sustained release of the therapeutic bi-functional surrogate antibody to the immediate area of the implant.

A variety of methods are available for delivering a surrogate antibody to a subject (i.e., a subject), tissue, organ, or cell). The manner of administering bi-functional surrogate antibodies for systemic delivery may be via subcutaneous, intramuscular, intravenous, ID, or intranasal. In addition inhalant mists, orally active formulations, transdermal iontophoresis or suppositories, are also envisioned. One carrier is physiological saline solution, but it is contemplated that other pharmaceutically acceptable carriers may also be used. In one embodiment, it is envisioned that the carrier and the surrogate antibody molecule constitute a physiologically-compatible, slow release formulation. The primary solvent in such a carrier may be either aqueous or non-aqueous in nature. In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption of the surrogate antibody. Such excipients are those substances usually and customarily employed to formulate dosages for parental administration in either unit dose or multi-dose form.

For example, in general, the disclosed bi-functional surrogate antibody can be incorporated within or on microparticles or liposomes. Microparticles or liposomes containing the disclosed surrogate antibody can be administered systemically, for example, by intravenous or intraperitoneal administration, in an amount effective for delivery of the disclosed bi-functional surrogate antibody to the ligand of interest. Other possible routes include trans-dermal or oral administration, when used in conjunction with appropriate microparticles. Generally, the total amount of the liposome-associated surrogate antibody administered to an individual will be less than the amount of the unassociated surrogate antibody that must be administered for the same desired or intended effect.

Thus the present invention also provides pharmaceutical formulations or compositions, both for veterinary and for human medical use, which comprise the a bi-functional surrogate antibody with one or more pharmaceutically acceptable carriers thereof and optionally any other therapeutic ingredients. The carrier(s) must be pharmaceutically acceptable in the sense of being compatible with the other ingredients of the formulation and not unduly deleterious to the recipient thereof.

The compositions include those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier or both, and then, if necessary, shaping the product into desired formulations.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parental administration conveniently comprise a sterile aqueous preparation of the active compound, which can be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the bi-functional surrogate antibody. The technology for forming liposomal suspensions is well known in the art. When the bi-functional surrogate antibody is an aqueous-soluble salt, using conventional liposome technology, the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone metabolite or salts thereof, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired surrogate antibody or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

In addition to the aforementioned ingredients, the compositions of the invention may further include one or more accessory ingredient(s) selected from the group consisting of diluents, buffers, flavoring agents, binders, disintegrants, surface active agents, thickeners, lubricants, preservatives (including antioxidants) and the like.

III. Kits

The disclosed bi-functional surrogate antibody molecules of the present invention can also be used as reagents in kits. The kit comprises a bi-functional surrogate antibody population having a attached thereto an agent capable of modulating an immune response and suitable buffers or carriers. In one example, the bi-functional surrogate antibody and the buffer can be present in the form of solutions, suspensions, or solids such as powders or lyophilisates. The reagents can be present together, separated from one another. The disclosed kit can also be used as a therapeutic agent.

Methods

The present invention provides bi-functional surrogate antibody molecules that interacts with a desired ligand of interest and further comprise an immunomodulatory agent that is capable of modulating an immune response. In this manner, interaction of the bi-functional surrogate antibody molecule with the target allows for a targeted immune response at the site of the ligand/surrogate antibody interaction.

A method of delivering an immunomodulatory agent to a ligand of interest is provided. This method comprises contacting the ligand with a bi-functional surrogate antibody. In some embodiments, the method comprises administering to a subject a composition comprising an isolated bi-functional surrogate antibody molecule comprising a specificity strand and a stabilization strand, wherein the specificity strand comprising a nucleic acid sequence having a specificity region flanked by a first constant region and a second constant region; the stabilization strand comprises a first stabilization domain that interacts with said first constant region and a second stabilization domain that interacts with said second constant region. The isolated bi-functional antibody further has attached thereto an immunomodulatory agent and the bi-functional surrogate antibody molecule is capable of interacting with the ligand of interest. In other embodiments, the stabilization strand and said specificity strand comprise distinct molecules.

Methods for assaying the interaction of the bi-functional surrogate antibody with the ligand of interest are known. For example, various methods of filtration and other routine techniques are known to measure binding which can be used to monitor ligand/surrogate antibody interactions. In addition, various techniques are known to allow one to determine an in-vivo interaction. For example, conjugation of the bi-surrogate antibody with a technetium-99m chelatin cage would enable in vivo imaging. See, for example, Hnatowich et al. (1998) Nucl. Med. 39:56-64. In addition, any functional moiety comprising a reporter molecules (i.e., radiolabel or fluorescent molecule) could be used to monitor the interaction.

The present invention further provides a method for modulating an immune response against a ligand in a subject. The method comprises administering to the subject an isolated bi-functional surrogate antibody molecule comprising a specificity strand and a stabilization strand, wherein the specificity strand comprising a nucleic acid sequence having a specificity region flanked by a first constant region and a second constant region; the stabilization strand comprises a first stabilization domain that interacts with the first constant region and a second stabilization domain that interacts with said second constant region. The bi-functional surrogate antibody further has attached thereto an immunomodulatory agent; and, the bi-functional surrogate antibody molecule is capable of interacting with the ligand of interest. Thus, the bi-functional surrogate antibodies find use as vaccine against a variety of disease, disorders and pathogens.

Such modulations of the immune response can be measured using standard bioassays including in vivo challenge assays, in vivo immunogenicity assays, in vitro cell receptor binding assays, and in vitro antigen contest assays. One of skill will recognize the appropriate assay for the intended application. For example, representative assays for the modulation of the complement response include assaying for the binding of the bi-functional surrogate antibody to C lq or assaying to determine if the bi-functional surrogate antibody has the ability to confer complement mediated cell lysis. See, for example, Duncan et al. (1988) Nature 332:738-40; U.S. Pat. No. 5,648,260; U.S. Pat. No. 5,624,821; Tao etal. (1993) J. Exp. Med. 178:661-667; Brekke etal. (1994) Eur. J. Immunol. 24:2542-47; Xu et al. (1993) J. Immunol. 150:152A; and, W094/29351; all of which are herein incorporated by reference.

Additional assays to measure the modulation of an immune response include an Elispot assay that measures vaccine-induced cellular immune responses. The assay measures the number of T-cells activated by a specific antigen. Briefly, a subject is challenged with the ligand of interest followed the administration of a bi-functional surrogate antibody. Responding cells are detected by staining for secreted (extracellular) cytokines. Other assays include intracellular cytokine assay (ICC). This assay measures the production of cytokines in response to a particular antigen. In this case, cytokines are detected inside the cells using fluorescent-labeled, cytokine-binding antibodies. Fluorescing cells are then counted using flow cytometry.

Additional assays include, assays to monitor binding to FcγR. See, Bredius et al. (1994) Immunology 83:624-630; Tax et al. (1984) J. Immunol. 133(3): 1185-1189; Nagarajan et al. (1995) J. Biol. Chem. 270(43):25762-25770; and Warmerdam et al. (1991) J. Immunol. 147(4):1338-1343, all of which are herein incorporated by reference.

Assays to monitor the half-life or clearance rate of the bi-functional surrogate antibody include assaying for direct interaction with FcRn or monitoring an increase or decrease in serum half-life, an increase in mean residence time in circulation (MRT), and/or a decrease in serum clearance rate over a surrogate antibody lacking the immune modulating agent. See, for example, U.S. Pat. No. 6,468,532, herein incorporated by reference. Assays for complement dependent cytotoxicity (CDC) can be preformed as described by Gazzano-Santoro et al. (1997) J. Immuno. Methods 202:163. See also, U.S. Pat. No. 6,194,551. Both of these references are herein incorporated by reference.

Further provided are methods for the treatment or prevention of various disorders. The method comprises administering to a subject in need thereof a composition comprising a therapeutically effective amount of an isolated bi-functional surrogate antibody molecule. The isolated bi-functional surrogate antibody comprises a specificity strand and a stabilization strand, wherein the specificity strand comprises a nucleic acid sequence having a specificity region flanked by a first constant region and a second constant region; the stabilization strand comprises a first stabilization domain that interacts with the first constant region and a second stabilization domain that interacts with said second constant region. The bi-functional surrogate antibody further has attached thereto an immunomodulatory agent; and, the bi-functional surrogate antibody molecule is capable of interacting with a ligand of interest.

By ” effective amount” is meant the concentration of a bi-functional surrogate antibody that is sufficient to elicit a modulation in the immune response (i.e., an increase or decrease in antibody-dependant cytotoxicity (ADCC), phagocytosis, complement-dependent cytotoxicity (CDC), and half-life/clearance rate). Thus, the effective amount of a bi-functional antibody will be sufficient to reduce or lessen the clinical systems of the disease, disorder, or conditions being treated or prevented.

The methods of the invention can be used alone, for example, to protect against or treat tumors, or can be used as adjuvant therapy following debulking of a tumor by conventional treatment such as surgery, radiotherapy and chemotherapy.

In other embodiments, the bi-functional surrogate antibody is delivered to mucosal surfaces of the subject. In this method, the bi-functional surrogate antibody has attached thereto a transporting agent.

In yet other embodiment, the present invention provides a method of inhibiting or preventing an infection prior to entry into the body. This method thereby offers a first line of defense prior to the entry of a particular pathogen into the subject. In one embodiment, a bi-functional surrogate antibody having a transport agent attached thereto can be used to produce a passive effect mechanism (e.g., blocking of viral receptors for host cells or inhibition of bacterial motions).

In other embodiments, the bi-functional surrogate antibody has attached thereto a transporting agent and at least one immunomodulaory agent and/or an anti-microbial agent and/or other therapeutic agent. Thus, an immunological response at the mucosal surface can be potentiated and thereby prevent the infective agent from entering the body. Such methods find use in the prevention of sexually transmitted diseases, maternal transmission of disease during birth, and prevention of other infections that enter though the mucosal surfaces such as the genitourinaery tract, mouth nasal passage, lungs, eyes, in man and domesticated and non-domesticated animals.

The concentration of a surrogate antibody in an administered dose unit in accordance with the present invention is effective to produce the desired effect. The effective amount will depend on many factors including, for example, the specific bi-functional surrogate antibody being used, the desired effect, the responsiveness of the subject, the weight of the subject along with other intrasubject variability, the method of administration, and the formulation used. Methods to determine efficacy, dosage, Ka, and route of administration are known to those skilled in the art.

An embodiment of the present invention provides for the administration of a bi-functional surrogate antibody in a dose of about 0.5 mg/kg, 1.0 mg/kg, 1.5 mg/kg, 2 mg/kg, 2.5 mg/kg, 3.0 mg/kg, 4.0 mg/kg, 5.0 mg/kg, 6.0 mg/kg, 15.0 mg/kg, 20 mg/kg. Alternatively, the surrogate antibody can be administered in a dose of about 0.2 mg/kg to 1.2 mg/kg, 1.2 mg/kg to 2.0 mg/kg, 2.0 mg/kg to 3.0 mg/kg, 3.0 mg/kg to 4 mg/kg, 4 mg/kg to 6 mg/kg, 6 mg/kg to 8 mg/kg, 8 mg/kg to 15 mg/kg, or 15 mg/kg to 20mg/kg.

In another embodiment of the invention, the pharmaceutical composition comprising the therapeutically effective dose of a bi-functional surrogate antibody is administered intermittently. By “intermittent administration” is intended administration of a therapeutically effective dose of a bi-functional surrogate antibody, followed by a time period of discontinuance, which is then followed by another administration of a therapeutically effective dose, and so forth. Administration of the therapeutically effective dose may be achieved in a continuous manner, as for example with a sustained-release formulation, or it may be achieved according to a desired daily dosage regimen, as for example with one, two, three, or more administrations per day. By “time period of discontinuance” is intended a discontinuing of the continuous sustained-released or daily administration of the regulatory agent. The time period of discontinuance may be longer or shorter than the period of continuous sustained-release or daily administration. During the time period of discontinuance, the bi-functional surrogate antibody level in the relevant tissue is substantially below the maximum level obtained during the treatment. The preferred length of the discontinuance period depends on the concentration of the effective dose and the form of bi-functional surrogate antibody used. The discontinuance period can be at least 2 days, at least 4 days, at least 1 week, or greater. When a sustained-release formulation is used, the discontinuance period must be extended to account for the greater residence time of regulatory agent at the site of injury. Alternatively, the frequency of administration of the effective dose of the sustained-release formulation can be decreased accordingly. An intermittent schedule of administration of bi-functional surrogate antibody can continue until the desired therapeutic effect, and ultimately treatment of the disease or disorder is achieved.

In yet another embodiment, intermittent administration of the therapeutically effective dose of regulatory agent is cyclic. By “cyclic” is intended intermittent administration accompanied by breaks in the administration, with cycles ranging from about 1 month to about 2, 3, 4, 5, or 6 months. For example, the administration schedule might be intermittent administration of the effective dose of bi-functional surrogate antibody, wherein a single short-term dose is given once per week for 4 weeks, followed by a break in intermittent administration for a period of 3 months, followed by intermittent administration by administration of a single short-term dose given once per week for 4 weeks, followed by a break in intermittent administration for a period of 3 months, and so forth. As another example, a single short-term dose may be given once per week for 2 weeks, followed by a break in intermittent administration for a period of 1 month, followed by a single short-term dose given once per week for 2 weeks, followed by a break in intermittent administration for a period of 1 month, and so forth. A cyclic intermittent schedule of administration of a regulatory agent to a subject may continue until the desired therapeutic effect, and ultimately treatment of the disorder or disease is achieved.

The present invention further provides a method for modulating the activity of the ligand of interest and modulating the immune response at the site of the ligand in a subject. The modulation of ligand could results from a direct interaction with the epitope binding domain of the bi-functional surrogate antibody. Alternatively, the bi-functional antibody can have attached thereto a functional moiety that is capable of modulating the activity of the target ligand or components in the vicinity of the target ligand.

Methods to assay for the modulation of ligand activity will vary depending on the ligand. One will further recognize the assay could directly measure ligand activity or alternatively, the phenotype of the cell, tissue or organ could be altered or the clinical outcome of the subject receiving the bi-functional surrogate antibody could be improved.

A functional agent capable of modulating the activity of the ligand can comprise a variety of therapeutic agents. Therapeutic agents of interest include, for example, those pharmaceutical compounds that are developed for use in the treatment of cancer, arthritis, septicemia, myocardial arrhythmia's and infarctions, viral and bacterial infections, autoimmune disease and prion diseases. In this manner, bi-functional surrogate antibodies can be used as a means to deliver a therapeutic agent and modulate a directed immune response in the region of the ligand.

When the therapeutic agent capable of modulating the activity of the ligand of interest is to be delivered to treat a particular disorder, the therapeutic agents can be selected for the particular disorder. For example, where the bi-surrogate antibodies are targeted to a unique ligand found on the surface of a tumor cell at a specific tumor site, the bi-functional surrogate antibodies can be conjugated to an anti-tumor agent for specific delivery to that site and to minimize or eliminate collateral pathology to normal tissue.

The therapeutic agents can be virtually any type of anti-tumor or anti-angiogenic compound (i.e., an agent that disrupts the vasculature supplying a tumor) that can be attached to the surrogate antibody, and can include, for purpose of example, synthetic or natural compounds such as cytotoxin, interleukins, chemotactic factors, radionucleotides, methotrexate, cis-platin, anastrozole/Arimidexg and tamoxifen.

Additional agents of interest include biological toxins such as ricin or diptheria toxin, fingal-derived calicheamicins, maytansinoids, momordin, pokeweek antiviral protein, Stapoloccoccal enterotoxin A, Pseudomanas exotoxins, ribosomes inactivating proteins and various cytotoxic drugs including neocarzinostatin, methotrexate, or callicheamicin. See, for example, Buschsbaum et al. (1999) Clin. Cancer Res 5: Grassband et al. (1992) Blood 79:576-83; Batra et al. (1991) Mol Cell Biol. 11:2200-5; Penichet et al. (2001) J ImmunolMeth 248:91-101; Hinman et al. (1993) Cancer Res 53:3336-3342; Tur et al. (2001) Intt JMol Med 8:579-584; Tazzari et al. (2001) J Immunol 167:4222-4229; Panousis et al. (1999) Drugs Aging 15:1-13, Trail et al. (1993) Science 261:212-5; Yamaguchi et al. (1993) Jpn J Cancer Res 84:1190-4.

Alternatively, the therapeutic agent could comprise a produg. After its localization to the specific target, a non-toxic molecule is injected that coverts the prodrug to a drug. See, for example, Senter et al. (1996) Advanced Drug Delivery 22:341-9.

In one embodiment, the functional moiety is a compound having anti-microbial activity. By “anti-microbial activity” is intended any ability to inhibit or decrease the growth of a microbe and/or the ability to decrease the number of microbes in a microbial population. By “microbe” in intended a bacterial, virus, fungi, or parasite and consequently, the functional moiety having anti-microbial activity possess anti-bacterial activity, anti-fungal activity, and/or anti-viral activity.

By “anti-bacterial activity” is intended any ability to inhibit or decrease the growth of a bacteria and/or the ability to decrease the number of viable bacterial cells in a bacterial population. The agent can be a Gram-positive anti-bacterial agent, a Gram-negative anti-bacterial agent, or a male specific anti-bacterial agent. By “anti-viral activity” is intended any ability to inhibit or decrease the growth of a virus or a virus infected cell and/or the ability to decrease the population of viable viral particles or virally infected cells in a population. The term “anti-fungal or mycotic activity” is intended the ability to inhibit or decrease the growth of fungi. Anti-microbial agents are known in the art and include various chemokines, cytokines, anti-microbial polypeptides (i.e., anti-bacterial, anti-viral, and anti-fungal polypeptides), antibiotics, LPS, complement activators, CpG sequence, and various other agents having anti-microbial activity. Exemplary anti-microbial agents are discussed in further detail below.

Accordingly, in one embodiment, the present invention provides a bi-functional surrogate antibody covalently attached to an anti-microbial agent. Using the various methods described herein, the bi-functional surrogate antibody can be designed to bind to a specific target ligand (i.e., an epitope of the target microbe). The bi-functional surrogate antibody/anti-microbial complex can then be used as a means to delivered the anti-microbial agent to the microbe, while the immunomodulatory agent will provide for a targeted immune response. Thus, the compositions find use as a therapeutic agent that, upon administration to a subject in need thereof, will inhibit or decrease the growth of a microbe contained within said subject and/or decrease the microbial population in the subject.

Examples of anti-microbial agents and their active variants and derivatives are known in the art and are disclosed in U.S. Application entitled “Surrogate Antibodies and Methods of Preparation and Uses Thereof” filed concurrently herewith and herein incorporated by reference.

In another embodiment, the bi-function surrogate antibodies potentiate an immune response in vitro. For example, in one embodiment, modulation of the immune response decreases the level of a microbe in a sample. In this embodiment, the ligand recognized by the surrogate antibody is a microbe (or a constituent on the surface of the microbe). The surrogate antibody is contacted with a population of cells and the bi-functional surrogate antibody interacts with the target microbe. The appropriate complement factors, neutrophiles, and/or lymphocytes are added to the sample. The appropriate complement factors, neutrophiles, or lymphocytes result in a targeted in vitro immune response and the microbes bound by the surrogate antibody are killed. Methods to assay for a decrease in microbe activity are known.

Generating a Surrogate Antibody

The bi-functional surrogate antibodies of the invention have attached thereto an immunomodulatory agent. Discussed below are methods for the production of a bi-functional surrogate antibody that interacts with a ligand having the desired specificity and affinity. It is recognized, that the immunomodulatory agent and/or transporting agent can be attached to the surrogate antibody at any of the selection steps discussed below. Therefore, while the below methods discuss “surrogate antibodies”, it is recognized that each population of surrogate antibody could also be (if one desired) a “bi-functional surrogate antibody” and therefore have attached thereto an immunomodulatory agent. The term “(bi-functional) surrogate antibody” is used in the methods described below to denote that either structure (a surrogate antibody or a bi-functional surrogate antibody) could be used.

I. (Bi-Functional) Surrogate Antibody Libraries

A surrogate antibody library or bi-functional surrogate antibody library can be screened to identify the (bi-functional) surrogate antibody or a population of (bi-functional) surrogate antibodies having the desired binding affinity and specificity to the ligand of interest. By “population” is intended a group or collection that comprises two or more (i.e., 10, 100, 1,000, 10,000, 1×10⁶, 1×10⁷, or 1×10⁸ or greater) (bi-functional) surrogate antibodies. Various “populations” of (bi-functional) surrogate antibodies exist and include, for example, a library of (bi-functional) surrogate antibodies, which as discussed in more detail below, comprises a population of (bi-functional) surrogate antibodies having a randomized specificity region. The various populations of (bi-functional) surrogate antibodies can be found in a mixture or in a substrate/array.

The binding diversity of (bi-functional) surrogate antibody molecules is not limited by the diversity of gene segments within the genome. Thus, a library of (bi-functional) surrogate antibody molecules can comprise molecules of diverse structure. For example, the size of the specificity domain can be varied in the population, thereby expanding the diversity of epitope dimensions that can be recognized. In addition, the diversity of the library is increased as function of the number of different nucleotide bases and functional moieties (i.e., nucleotide modifications). A library having a specificity region composed of 40 natural nucleotides potentially has 1.2×10²⁴ specificities. The production of (bi-functional) surrogate antibody molecules having multiple specificity regions increases this number. The selective use of modified bases in conjunction with natural bases again increases the diversity of the antibody repertoire.

The library of (bi-functional) surrogate antibodies progresses through a series of iterative in vitro selection techniques that allow for the identification/capture of the desired (bi-functional) surrogate antibody(ies). Each round of selection produces a selected population of (bi-functional) surrogate antibody molecules that have an increased binding affinity and/or specificity to the desired ligand as compared to the library. See, for example, U.S. Application entitled “Surrogate Antibodies and Methods of Preparation and Uses Thereof” filed concurrently herewith and herein incorporated by reference.

A library of (bi-functional) surrogate antibody molecules is a mixture of stable, preformed, (bi-functional) surrogate antibody molecules of differing sequences, from which (bi-functional) surrogate antibody molecules able to bind a desired ligand are captured. As used herein, a library of (bi-functional) surrogate antibody molecules comprises a population of molecules comprising a specificity strand and a stabilization strand. The specificity strand comprises a nucleic acid sequence having a specificity region flanked by a first constant region and a second constant region; and, the stabilization strand comprises a first stabilization domain that interacts with said first constant region and a second stabilization domain that interacts with said second constant region. In addition, each of the first constant regions of the specificity strands in the population are identical; each of the second constant regions of the specificity strands in the population are identical; each of the specificity region of the specificity strands in said population are randomized; and, each of the stabilization strands in said population are identical. It is recognized that a library of (bi-functional) surrogate antibody molecules having any of the diverse structures, described elsewhere herein, can be assembled.

As used herein, a library typically includes a population having between about 2 and about 1×10¹⁴ (bi-functional) surrogate antibodies. Alternatively, the (bi-functional) surrogate antibody library used for selection can include a mixture of between about 2 and about 10¹⁸, between about 10⁹ and about 10¹⁴, between about 2 and 10²⁷ or greater (bi-functional) surrogate antibodies having a contiguous randomized sequence of at least 10 nucleotides in length in each binding cavity (i.e., specificity domain). In yet other embodiments, the library will comprise at least 3, 10, 100, 1000, 10000, 1×10⁵, 1×10⁶, 1×10⁷, 1×10¹⁰, 1×10¹⁴, 1×10¹⁸, 1×10²², 1×10²⁵, 1×10²⁷ or greater (bi-functional) surrogate antibody molecules having a randomized or semi-random specificity domain. The molecules contained in the library can be found together in a mixture or in an array.

In certain other instances of usage herein, the term “population” may be used to refer to polyclonal or monoclonal surrogate antibody preparations of the invention having one or more selected characteristics.

A “population of polyclonal antibodies” comprises a population of individual clones of (bi-functional) surrogate antibodies assembled to produce polyclonal libraries with enhanced binding to a ligand of interest. Once a (bi-functional) surrogate antibody, or a plurality of separate (bi-functional) surrogate antibody clones, are found to meet target performance criteria they can be assembled into polyclonal reagents that provide multiple epitope recognition and greater sensitivity in detecting/interacting with the target ligand. It is recognized that a population of polyclonal surrogate antibodies can represent a pool of molecules obtained following the capture and amplification steps to a desired ligand. Alternatively, a population of polyclonal surrogate antibodies could be formed by mixing at least two individual monoclonal (bi-functional) surrogate antibody clones having the desired ligand binding characteristics.

A. Forming the Randomized Population of Specificity Regions

Methods of producing or forming a population of specificity strands having randomized specificity domains are known in the art. For example, the specificity region(s) can be prepared in a number of ways including, for example, the synthesis of randomized nucleic acid sequences and selection from randomly cleaved cellular nucleic acids. Alternatively, full or partial sequence randomization can be readily achieved by direct chemical synthesis of the nucleic acid (or portions thereof) or by synthesis of a template from which the nucleic acid (or portions thereof) can be prepared by using appropriate enzymes. See, for example, Breaker et al. (1997) Science 261:1411-1418; Jaeger et al. (1997) Methods Enzy 183:281-306; Gold et al. (1995) Annu Rev Biochem 64:763-797; Perspective Biosystems (1998) and Beaucage et al. (2000) Current Protocols in Nucleic Acid Chemistry John Wily & Sons, New York 3.3.1-3.3.20; all of which are herein incorporated by reference. Alternatively, the oligonucleotides can be cleaved from natural sources (genomic DNA or cellular RNA preparations) and ligated between constant regions.

Randomized is a term used to describe a segment of a nucleic acid having, in principle, any possible sequence of nucleotides containing natural or modified bases over a given length. As discussed above, the specificity region can be of various lengths. Therefore, the randomized sequences in the (bi-functional) surrogate antibody library can also be of various lengths, as desired, ranging from about ten to about 90 nucleotides or more. The chemical or enzymatic reactions by which random sequence segments are made may not yield mathematically random sequences due to unknown biases or nucleotide preferences that may exist. The term “randomized” or “random,” as used herein, reflects the possibility of such deviations from non-ideality. In the techniques presently known, for example sequential chemical synthesis, large deviations are not known to occur. For short segments of 20 nucleotides or less, any minor bias that might exist would have negligible consequences. The longer the sequences of a single synthesis, the greater the effect of any bias.

Sequence variability (i.e., library diversity) can be achieved using size-selected fragments of partially digested (or otherwise cleaved) preparations of large, natural nucleic acids, such as genomic DNA preparations or cellular RNA preparations. It is not necessary that the library includes all possible variant sequences. The library can include as large a number of possible sequence variants as is practical for selection, to insure that a maximum number of potential binding sequences are identified. For example, if the randomized sequence in the specificity region includes 30 nucleotides, it would contain approximately 1018 (i.e. 430) sequence permutations using the 4 naturally occurring bases.

A bias can be deliberately introduced into randomized sequence, for example, by altering the molar ratios of precursor nucleoside (or deoxynucleoside) triphosphates of the synthesis reaction. A deliberate bias may be desired, for example, to approximate the proportions of individual bases in a given organism or to affect secondary structure. See, Hermes et al. (1998) Gene 84:143-151 and Bartel et al. (1991) Cell 67:529-536, both of which are herein incorporated by reference. See also, Davis et al. (2002) Proc. Natl. Acad. Sci. 99:11616-11621, which generated a randomized population having a bias comprising a specified stem loop structure. Thus, as used herein, a randomized population of specificity domains may be generated to contain a desirable bias in the primary sequence and/or secondary structure of the domain.

In other embodiments, the length of the specificity region of individual members within the library can be substantially the same or different. Iterative libraries can be used, where the specificity domain varies in size in each library or are combined to form a library of mixed loop sizes, for the purpose of identifying the optimum loop size for a particular target ligand.

As discussed above, the specificity strand may contain various functional moieties. Methods of forming the randomized population of specificity strands will vary depending on the functional moieties that are to be contained on the strand. For example, in one embodiment, the functional moieties comprise modified adenosine residue. In this instance, the specificity strand could be designed to contain adenosine residues only in the specificity domain. The nucleotide mixture used upon amplification will contain the adenosine having the desired functional moieties (i.e., moieties that increase hydrophobic binding characteristics). In other instances, the functional moiety can be attached to the surrogate antibody following the synthesis reaction.

The agent capable of modulating an immune response can be attached to the antibody at anytime during the selection process alternatively, the agent can be attached following the identification of a surrogate antibody having the desired ligand binding characteristics.

B. Generating a (Bi-Functional) Surrogate Antibody Library

Once the population of specificity strands having a randomized assortment of specificity regions has been formed, the (bi-functional) surrogate antibodies are formed (as discussed elsewhere herein) by contacting the specificity strand with an appropriate stabilization strand under the desired conditions.

Generating a library of (bi-functional) surrogate antibody molecule comprises: a) providing a population of specificity strands wherein i) the population of specificity strands is characterized as a population of nucleic acid molecules; ii) each of the specificity strands in said population comprises a nucleic acid sequence having a specificity region flanked by a first constant region and a second constant region; iii) each of the first constant region of the specificity strands in the population are identical; iv) each of the second constant region of the specificity strands in said population are identical; and, v) each of the specificity regions of said specificity strands in said population are randomized. The population of specificity strands is contacted with a stabilization strand; wherein the stabilization strand comprises a first stabilization domain that interacts with said first constant region and a second stabilization domain that interacts with said second constant region, wherein said contacting occurs under conditions that allow for the first stabilization domain to interact with the first constant region and the second stabilization domain to interacts with the second constant region. In other embodiments surrogate antibodies that compose the library have a specificity strand and a stabilization strand contained on distinct strands.

As discussed above, it may be beneficial to produce a population of (bi-functional) surrogate antibodies having a randomized specificity domain that varies in length. In this manner, the library could be used in a “multi-fit” process of (bi-functional) surrogate antibody development that defines the optimal surrogate antibody cavity size to use for any given ligand. The process allows surrogate antibody binding to improve upon the binding characteristics of native antibody molecules where the size of the paratope (binding site) is finite for all ligands regardless of size. The “multi-fit” process identifies a cavity size with spatial characteristics that enhance the fit, specificity, and affinity of the surrogate antibody-ligand complex. The “multi-fit” process can identify as an ideal binding loop/cavity one that is not restricted in size or dimensionality by the precepts of evolution and genetics. As such, surrogate antibody molecules challenge the conventional paradigm regarding the size of an epitope or determinant as shaped by the dependency of science and research on the properties of native antibody molecules. Preliminary “multi-fit” ligand capture rounds are performed using a heterogeneous population of surrogate antibodies containing specificity domains of varying size and conformation. The optimal cavity size for surrogate library preparation is indicated by the sub-population having a cavity size that exhibits the highest degree of ligand binding after a limited number of capture and amplification cycles.

C. Methods of Screening a (Bi-Functional) Surrogate Antibody Library

The (bi-functional) surrogate antibody library or a selected population of (bi-functional) surrogate antibodies can be screened to identify or “capture” a (bi-functional) surrogate antibody or a population of (bi-functional) surrogate antibodies having the desired ligand-binding characteristics. In this manner, (bi-functional) surrogate antibody molecules are selected for subsequent cloning from a library of pre-synthesized multi-stranded molecules that contain a random specificity region and stabilization regions that stabilize the structure of the molecule in solution.

Generally, (bi-functional) surrogate antibodies that bind to a particular ligand are captured from a starting surrogate antibody library by contacting one or more ligand with the library, binding one or more (bi-functional) surrogate antibodies to the ligand(s), separating the (bi-functional) surrogate antibody bound ligand from unbound (bi-functional) surrogate antibody, and identifying the bound ligand and/or the bound (bi-functional) surrogate antibodies.

For example, a method for screening a (bi-functional) surrogate antibody library comprises:
a) contacting at least one ligand of interest with a library of (bi-functional) surrogate antibody molecules, said library comprising a population of (bi-functional) surrogate antibody molecules comprising a specificity strand and a stabilization strand; wherein,
   i) the specificity strand comprises a nucleic acid sequence having a specificity region flanked by a first constant region and a second constant region; and, the stabilization strand comprises a first stabilization domain that interacts with said first constant region and a second stabilization domain that interacts with said second constant region;
   ii) each of the first constant regions of the specificity strands in the population are identical; each of the second constant region of the specificity strands in the population are identical; each of the specificity domains of the specificity strands in said population are randomized; and, each of the stabilization strands in said population are identical;
b) partitioning the target ligand and the population of (bi-functional) surrogate antibody molecules from the population of ligand-bound (bi-functional) surrogate antibody complexes; and,
c) amplifying the specificity strand of the population of ligand-bound (bi-functional) surrogate antibody complexes.

In still other embodiments, the method of screening a (bi-functional) surrogate antibody library further comprises contacting the population of specificity strands of step (c) with a stabilization strand under conditions that allow for the first stabilization domain to interact with the first constant region and said second stabilization domain to interact with said second constant region.

In other embodiments, the stabilization strand and the specificity strand of the (bi-functional) surrogate antibody molecules are distinct.

As discussed previously, the methods allow for the selection or capturing of a (bi-functional) surrogate antibody molecule that interacts with the desired ligand of interest. The method thereby employs selection from a library of (bi-functional) surrogate antibody molecules followed by step-wise repetition of selection and amplification to allow for the identification of the (bi-functional) surrogate antibody molecule that have the desired binding affinity and/or selectivity for the ligand of interest. As used herein a “selected population of (bi-functional) surrogate antibody molecules” is intended a population of molecules that have undergone at least one round of ligand binding.

Accordingly, in another embodiment, the method of capturing a (bi-functional) surrogate antibody comprises contacting a selected population of (bi-functional) surrogate antibodies with the ligand of interest. In this embodiment, a library of molecules containing a randomized specificity domain need not be use, but rather a selected population of (bi-functional) surrogate antibody molecules generated, for example, following the second, third, fourth, fifth, sixth, seventh or higher round of selection/amplification could be contacted with the desired ligand. In this embodiment, a method for capturing a (bi-functional) surrogate antibody comprises:
a) contacting a ligand with a population of (bi-functional) surrogate antibody molecules under conditions that permit formation of a population of ligand-bound (bi-functional) surrogate antibody complexes, wherein said (bi-functional) surrogate antibody molecule of the (bi-functional) surrogate antibody population comprises a specificity strand and a stabilization strand,
   said specificity strand comprising a nucleic acid sequence having a specificity region flanked by a first constant region and a second constant region; and,
   said stabilization strand comprises a first stabilization domain that interacts with said first constant region and a second stabilization domain that interacts with said second constant region;
b) partitioning the ligand and the population of (bi-functional) surrogate antibody molecules from said population of ligand-bound (bi-functional) surrogate antibody complexes; and,
c) amplifying the specificity strand of said population of ligand-bound (bi-functional) surrogate antibody complexes.

In other embodiments, the method of capturing a surrogate antibody molecule further comprises contacting the population of specificity strands of step (c) with a stabilization strand under conditions that allow for the first stabilization domain to interact with the first constant region and the second stabilization domain to interact with said second constant region. In yet other embodiments, the stabilization strand and the specificity strand are distinct.

It is recognized that in the various methods described above, more than one target ligand can be used to simultaneously capture a plurality of (bi-functional) surrogate antibodies from a starting library or population or to enhance binding specificity of the population of antibodies.

i. Methods of Contacting:

By “contacting” is intended any method that allows a desired ligand of interest to interact with a (bi-functional) surrogate antibody molecule or a population thereof. One of skill in the art will recognize that a variety of conditions could be used for this interaction. For example, the experimental conditions used to select (bi-functional) surrogate antibodies that bind to various target ligands can be selected to mimic the environment that the target would be found in vivo or the anticipated in vitro application. Adjustable conditions that can be altered to more accurately reflect this binding environment include, but are not limited to, total ionic strength (osmolarity), pH, enzyme composition (e.g. nucleases), metalloproteins (e.g. hemoglobin, ceruloplasm), temperature, and the presence of irrelevant compounds. See, for example, Dang et al. (1996) J Mol Bio 264:268-278; O'Connell et al. (1996) Proc. Natl Acad Sci USA 93:5883-7; Bridonneu et al. (1999) Antisense Nucleic Acid Drug Dev 9:1-11; Hicke et al. (1996) J. Clin Investig 98:2688-92; and, Lin et al. (1997) J Mol Biol 271:446-8, all of which are herein incorporated by reference. Appropriate physiological conditions have been described in greater detail elsewhere herein.

Appropriate conditions to contact the ligand of interest and the surrogate antibody can be determined empirically based on the reaction chemistry. In general, the appropriate conditions will be sufficient to allow 1% to 5%, 5%-1 0%, 10% to 20%, 20% to 40%, 40% to 60%, 60% to 80%, 80% to 90%, or 90% to 100% of the antibody molecule population to interact with the ligand. One of skill will recognize the appropriate conditions based on the desired outcome (i.e., interaction with ligand, specificity enhancement, affinity enhancement, ect.).

ii. Methods of Partitioning:

By “partitioning” is intended any process whereby (bi-functional) surrogate antibody bound to target ligand, termed ligand-bound (bi-functional) surrogate antibody complexes, are separated from (bi-functional) surrogate antibodies not bound to target ligands. Partitioning can be accomplished by various methods known in the art. For example, surrogate antibodies bound to ligands of interest can be immobilized, or fail to pass through filters or molecular sieves, while unbound surrogate antibodies are not. Columns that specifically retain ligand-bound (bi-functional) surrogate antibody can be used for partitioning. Liquid-liquid partition can also be used as well as filtration gel retardation, and density gradient centrifugation. The choice of the partitioning method will depend on properties of the ligand and on the ligand-bound (bi-functional) surrogate antibody and can be made according to principles and properties known to those of ordinary skill in the art.

In one embodiment, partitioning comprises filtering a mixture comprising the ligand of interest, the population of (bi-functional) surrogate antibody molecules, and the population of ligand-bound (bi-functional) surrogate antibody complexes through a filtering system wherein said filtering system is characterized as allowing for the retention of the ligand-bound (bi-functional) surrogate antibody complex in the retentate and allowing the unbound (bi-functional) surrogate antibodies to pass into the filtrate. Such filtering systems are known in the art. For example, various filtration membranes can be used. The term “filtration membrane” includes devices that separate on the basis of size (e.g. Amicon Microcon®, Pall Nanosep®)), charge, hydrophobicity, chelation, and clathration.

The pore size used in the filtration process can be paired to the size of the target ligand and size of the (bi-functional) surrogate antibody molecule used in the initial population of (bi-functional) surrogate antibodies. For example, a cellular-ligand having a 7-10 micron diameter will be retained by a membrane that excludes 7 microns. (Bi-functional) surrogate antibody molecules having a 120 nucleotide bi-oligonucleotide structure when uncomplexed are easily eliminated as they pass through the membrane. Those bound to the ligand are captured in the retentate and used for assembly of the subsequent population. The preparation of a (bi-functional) surrogate antibody to a BSA-hapten conjugate must use a pore that excludes the surrogate antibody-conjugate complex. A membrane that excludes 50,000 or 100,000 daltons effectively fractionates this (bi-functional) surrogate antibody when bound to the conjugate from free (bi-functional) surrogate antibody. (Bi-functional) surrogate antibody prepared to a small protein, such as the enzyme Horseradish Peroxidase requires a membrane that would exclude molecules that are approximately 50,000 daltons or greater, while allowing the uncomplexed (bi-functional) surrogate antibody to penetrate the filter. The ligand of interest can be chemically conjugated to larger carrier molecules or polymerized to enhance their size and membrane exclusion characteristics.

Alternative protocols used to separate (bi-functional) surrogate antibodies bound to target ligands from unbound(bi-functional) surrogate antibody[ies] are available to the art. For example, the separation of ligand-bound and free (bi-functional) surrogate antibody molecules that exist in solution can be achieved using size exclusion column chromatography, reverse phase chromatography, size exclusion/molecular sieving filtering, affinity chromatography, electrophoretic methods, ion exchange chromatography, solubility modification (e.g. ammonium sulfate or methanol precipitation), immunoprecipitation, protein denaturation, FACS density gradient centrifugation. Ligand-bound and unbound (bi-functional) surrogate antibody molecules can be separated using analytical methods such as HPLC and fluorescent activated cell sorters.

Affinity chromatography procedures using selective immobilization to a solid phase can be used to separate (bi-functional) surrogate antibody bound to a target ligand from unbound (bi-functional) surrogate antibody molecules. Such methods could include immobilization of the target ligand onto absorbents composed of agarose, polyethylene, polystyrene, dextran, polyacrylamide, glass, nylon, cellulose acetate, polypropylene, or silicone chips.

Method of amplifying the specificity strand of the (bi-functional) surrogate antibody are described below, however, it is recognized that a surrogate antibody bound to the target ligand could be used in PCR amplification to produce oligonucleotide strand(s) having an integral specificity region(s) with or without separation from the affinity matrix. (Bi-functional) surrogate catalytic antibodies can be selected, based on binding affinity and the catalytic activity of the antibodies once bound. One way to select for catalytic antibodies is to search for surrogate antibodies that bind to transition state analogs of an enzyme catalyzed reaction.

A combination of solution and solid-phase separation could include binding a (bi-functional) surrogate antibody to ligand conjugated microspheres that could be isolated based upon a physicochemical effect created by the (bi-functional) surrogate antibody binding. Separate microsphere populations could individually be labeled with chromophores, fluorophores, magnetite conjugated to different target ligands or difference orientations of the same ligand. (Bi-functional) surrogate antibody molecules bound to each microsphere population could be isolated on the basis of microsphere reporter molecule characteristic(s), allowing for production of multiple surrogate populations to different ligands simultaneously.

The methods can be used to simultaneously produce (bi-functional) surrogate antibody molecules that bind to multiple, chemically distinct ligands. For example, the method can be used to select (bi-functional) surrogate antibodies for a mixed population of target ligand conjugates unable to penetrate the membrane. Sequential incubation of a surrogate antibody population with un-conjugated filterable ligand allows for separation of non-specific (bi-functional) surrogate antibody populations in the filtrate. Pre-incubation with filterable target ligands allows for rapid fractionation of (bi-functional) surrogate antibody populations in the retentate for subsequent amplification.

iii. Methods of Amplifying

Methods for amplifying the specificity strand of a (bi-functional) surrogate antibody molecule, amplifying the specificity strands a population of (bi-functional) surrogate antibodies, and/or amplifying the specificity strand(s) of a ligand-bound (bi-functional) surrogate antibody complex are provided. Amplifying or amplification means any process or combination of process steps that increases the amount or number of copies of a molecule or class of molecules. RNA molecules can be amplified by a sequence of three reactions: making cDNA copies of selected RNAs, using polymerase chain reaction to increase the copy number of each cDNA, and transcribing the cDNA copies to obtain RNA molecules having the same sequences as the selected RNAs. Any reaction or combination of reactions known in the art can be used as appropriate, including direct DNA replication, direct RNA amplification and the like, as will be recognized by those skilled in the art. The amplification method should result in the proportions of the amplified mixture being essentially representative of the proportions of different constituent sequences in the initial mixture. While the constant regions on either side of the specificity region in the (bi-functional) surrogate antibody molecule stabilize the structure of the specificity region, these regions can also be used to facilitate the amplification of the (bi-functional) surrogate antibodies.

In this manner, a population of specificity strands is generated. Thus, when the amplified specificity strands are contacted with the appropriate stabilization stand, a population of (bi-functional) surrogate antibodies having the desired ligand binding affinity and/or specificity can be formed. Methods to selectively enhance the specificity of the ligand interaction and methods for enhancing the binding affinity of the population are provided below.

Once a desired (bi-functional) surrogate antibody or set of surrogate antibodies is identified, it is often desirable to identify one or more of the monoclonal (bi-functional) surrogate antibody clones and generate large amount of either a monoclonal or assembled polyclonal (bi-functional) surrogate antibody reagent. Capturing a monoclonal (bi-functional) surrogate antibody comprises cloning at least one specificity strand from the population of amplified specificity strands. The cloned specificity strand can be amplified using routine methods and subsequently contacted with the appropriate stabilization strand under conditions that allow for said first stabilization domain to interact with said first constant region and said second stabilization domain to interact with said second constant region, and thereby producing a population of monoclonal (bi-functional) surrogate antibodies.

Methods of amplifying nucleic acid sequences (such as those of the specificity strand) are known. Polymerase chain reaction (PCR) is an exemplary method for amplifying nucleic acids. PCR methods are described, for example in Saiki et al. (1985) Science 230:1350-1354; Saiki et al. (1986) Nature 324:163-166; Scharf et al. (1986) Science 233:1076-1078; Innis et al. (1988) Proc. Natl. Acad. Sci. 85:9436-9440; U.S. Pat. No. 4,683,195; and, U.S. Pat. No. 4,683,202, the contents of each of which are incorporated herein in their entirety.

PCR amplification involves repeated cycles of replication of a desired single-stranded DNA (or cDNA copy of an RNA) employing specific oligonucleotide primers complementary to the 3′ and 5′ ends of the ssDNA, primer extension with a DNA polymerase, and DNA denaturation. Products generated by extension from one primer serve as templates for extension from the other primer. A related amplification method described in PCT published application WO 89/01050 requires the presence or introduction of a promoter sequence upstream of the sequence to be amplified, to give a double-stranded intermediate. Multiple RNA copies of the double-stranded promoter containing intermediate are then produced using RNA polymerase. The resultant RNA copies are treated with reverse transcriptase to produce additional double-stranded promoter containing intermediates that can then be subject to another round of amplification with RNA polymerase. Alternative methods of amplification include among others cloning of selected DNAs or cDNA copies of selected RNAs into an appropriate vector and introduction of that vector into a host organism where the vector and the cloned DNAs are replicated and thus amplified (Guatelli et al. (1990) Proc. Natl. Acad. Sci. 87:1874). In general, any means that will allow faithful, efficient amplification of selected nucleic acid sequences can be used. It is only necessary that the proportionate representation of sequences after amplification at least roughly reflect the relative proportions of sequences in the mixture before amplification. See, also, Crameri et al. (1 993) Nucleic Acid Research 21: 4110, herein incorporated by reference.

The method can optionally include appropriate nucleic acid purification steps.

(Bi-functional) surrogate antibody strands that contain specificity region nucleotides will generally be capable of being amplified. Generally, any conserved regions used in this strand also will not include molecules that interfere with amplification. However, functional moieties can be introduced, e.g. via selective chemistry, to the stabilization strand that may interfere with amplification of this strand by methods such as PCR. Such surrogate antibodies can be produced by any necessary biological and/or chemical steps in accordance with the methods of the invention.

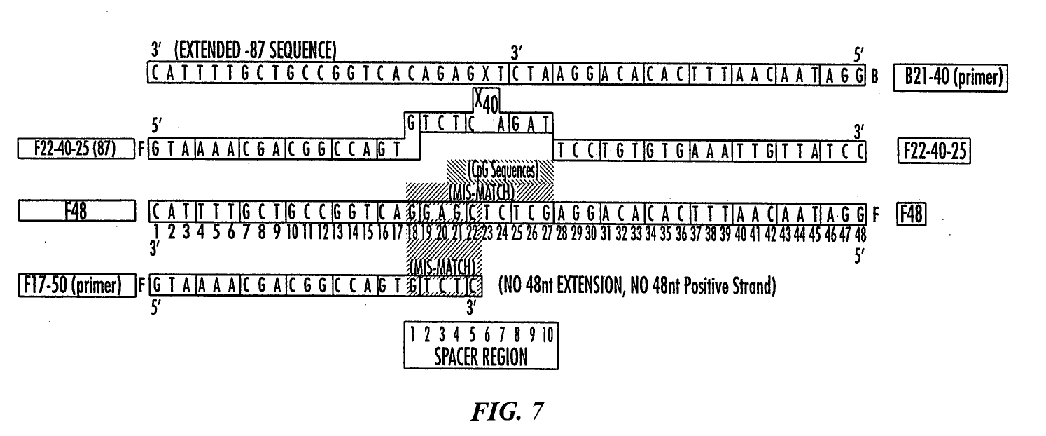

In other embodiments, the stabilization strand and the specificity strand contain a region of non-homology that can be used, in combination with the appropriate primers, to prevent the amplification of the stabilization strand. A non-limiting example of this embodiment appears in FIG. 7 and in Example 1 of the Experimental section. Briefly, in this non-limiting example, the stabilization strand and specificity strand lack homology in about 2, 3, 4, 5, 6, 8 or more nucleotides positioned 5′ to the specificity domain. See, shaded box in FIG. 7. The primer used to amplify the positive strand of the specificity strand is complementary to the sequences of the specificity strand. However, due to the mis-match design, this primer lacks homology at its 3′ end to the sequence of the stabilization strand. This lack of homology prevents amplification of the full-length negative stabilization strand. This method therefore allows for the preferential amplification of the specificity strand.

iv. Staging

The process of iterative selection of (bi-functional) surrogate antibody elements that specifically bind to a selected ligand of interest with high affinity is herein designated “staging.” Staging is a term that implies the “capture and amplification” of (bi-functional) surrogate antibody molecules that bind a target ligand that can be macromolecular or the size of an immunological hapten. The staging process can be modified in various ways to allow for this identification of the desired (bi-functional) surrogate antibody. For instance, steps can be taken to allow for “specificity enhancement” and thereby eliminate or reduce the number of irrelevant or undesirable (bi-functional) surrogate antibody molecules from the captured population. In addition, “affinity enhancement” can be performed and thereby allow for the selection of high affinity (bi-functional) surrogate antibody molecules to the target ligand. The staging process is particularly useful in the rapid isolation and amplification of (bi-functional) surrogate antibodies that have high affinity and specificity for the target ligand of interest. See, for example, Crameri et al. (1993) Nucleic Acid Research 21:4410.

Specific binding is a term that is defined on a case-by-case basis. In the context of a given interaction between a given (bi-functional) surrogate antibody molecule and a given ligand, enhanced binding specificity results when the preferential binding interaction of a (bi-functional) surrogate antibody with the target is greater than the interaction observed between the (bi-functional) surrogate antibody and irrelevant and/or undesirable targets. The (bi-functional) surrogate antibody molecules can be selected to be as specific as required using the “staging” process to capture, isolate, and amplify specific molecules.

Accordingly, a method of enhancing the binding specificity of a (bi-functional) surrogate antibody comprises:
a) contacting a population of (bi-functional) surrogate antibody molecules, said population of (bi-functional) surrogate antibody molecules capable of binding a ligand of interest, with a non-specific moiety under conditions that permit formation of a population of non-specific moiety-bound (bi-functional) surrogate antibody complexes,
wherein said surrogate antibody molecule of the surrogate antibody population comprises a specificity strand and a stabilization strand, said specificity strand comprising a nucleic acid sequence having a specificity region flanked by a first constant region and a second constant region; and, said stabilization strand comprises a first stabilization domain that interacts with said first constant region and a second stabilization domain that interacts with said second constant region;
b) partitioning said non-specific moiety and said population of non-specific moiety-bound (bi-functional) surrogate antibody complexes from said population of unbound (bi-functional) surrogate antibodies molecules; and,
c) amplifying the specificity strand of the population of unbound (bi-functional) surrogate antibody molecules.

The method of enhancing the binding affinity can further comprises contacting the population of specificity strands of step (c) above with a stabilization strand under conditions that allow for said first stabilization domain to interact with said first constant region and said second stabilization domain to interact with said second constant region.

In further embodiments, the population of (bi-functional) surrogate antibodies comprises a library of (bi-functional) surrogate antibodies and/or a population of selected (bi-functional) surrogate antibodies.

The binding specificity of the (bi-functional) surrogate antibody population is enhanced by contacting the population of (bi-functional) surrogate antibodies with a non-specific moiety under conditions that permit formation of a population of non-specific moiety-bound (bi-functional) surrogate antibody complexes. In this manner, (bi-functional) surrogate antibodies that interact with both the target ligand and a variety of non-specific moieties can partitioned from the population of (bi-functional) surrogate antibodies having a higher level of specificity to the desired ligand.

By “non-specific moiety” is intended any molecule, chemical compound, cell, organism, virus, nucleotide, or polypeptide that is not the desired target ligand. Depending on the desired surrogate antibody population being produced, one of skill in the art will recognize the most appropriate non-specific moiety to be used. For example, if the desired target is protein X which has 95% sequence identity to protein Y, the binding specificity of the (bi-functional) surrogate antibody population to protein X could be enhanced by using protein Y as a non-specific moiety. In this way, a (bi-functional) surrogate antibody population with enhanced interaction to protein X could be produce. See, for example, Giver et al. (1993) Nucleic Acid Research 23: 5509-5516 and Jellinek etal. (1993) Proc. Natl. Acad. Sci90:11227-11231.

Binding affinity is a term that describes the strength of the binding interaction between the (bi-functional) surrogate antibody and a ligand. An enhancement in binding affinity results in the increased binding interaction between the target ligand and the (bi-functional) surrogate antibody. The binding affinity of the (bi-functional) surrogate antibody and target ligand interaction directly correlates to the sensitivity of detection that the (bi-functional) surrogate antibody will be able to achieve. In order to assess the binding affinity under practical applications, the conditions of the binding reactions must be comparable to the conditions of the intended use. For the most accurate comparisons, measurements will be made that reflect the interaction between the (bi-functional) surrogate antibody and target ligand in solutions and under conditions of their intended application.

Accordingly, the present invention provides method of enhancing the binding affinity of a (bi-functional) surrogate antibody comprising:
a) contacting a ligand with a population of (bi-functional) surrogate antibody molecules under stringent conditions that permit formation of a population of ligand-bound (bi-functional) surrogate antibody complexes,
wherein said (bi-functional) surrogate antibody molecule of the (bi-functional) surrogate antibody population comprises a specificity strand and a stabilization strand,
said specificity strand comprising a nucleic acid sequence having a specificity region flanked by a first constant region and a second constant region; and,
said stabilization strand comprises a first stabilization domain that interacts with said first constant region and a second stabilization domain that interacts with said second constant region;
b) partitioning the ligand, said population of (bi-functional) surrogate antibody molecules from said population of ligand-bound (bi-functional) surrogate antibody complexes; and,
c) amplifying the specificity strand of said population of ligand-bound (bi-functional) surrogate antibody complexes.

In a further embodiment, the method of enhancing binding affinity further comprises contacting said population of specificity strands of step (c) above with a stabilization strand under conditions that allow for said first stabilization domain to interact with said first constant region and said second stabilization domain to interact with said second constant region.

In further embodiments, the population of (bi-functional) surrogate antibodies comprises a library of (bi-functional) surrogate antibodies and/or a population of selected (bi-functional) surrogate antibodies.

In this embodiment, contacting the desired ligand with a population of (bi-functional) surrogate antibody molecules under stringent conditions that permit formation of a population of ligand-bound (bi-functional) surrogate antibody complexes, allows for the selection of (bi-functional) surrogate antibodies that have increased binding affinity to the desired ligand. By “stringent conditions” is intended any condition that will stress the interaction of the desired ligand with the (bi-functional) surrogate antibodies in the population. Such conditions will vary depending on the ligand of interest and the preferred conditions under which the (bi-functional) surrogate antibody and ligand will interact. It is recognized that the stringent condition selected will continue to allow for the formation of the surrogate antibody structure. Examples of such stringent conditions include changes in osmolarity, pH, solvent (organic or inorganic), temperature, or any combination thereof. Additional components could produce stringent conditions include components that compromise hydrophobic, hydrogen bonding, electrostatic, and Van der Waals interactions. For example, 10% methanol or ethanol compromise hydrophobic boning and are water soluble.

The stringency of conditions can also be manipulated by the (bi-functional) surrogate antibody to ligand ratio. For example, following a few rounds of selection using equal (bi-functional) surrogate antibody: ligand ratio, the ratio can be increased to 1:10 or 1:100. This increase can occur by an increase in (bi-functional) surrogate antibody or by a decrease in target ligand. See, for example Irvine et al. (1991) J Mol Biol 222:739-761. Additional alterations to increase the stringency of binding conditions include, alterations in salt concentration, binding equilibrium time, dilution of binding buffer and amount and composition of wash. The stringency of conditions will be sufficient to decrease % antibody bound by 1% to 10%, 10% to 20%, 20% to 30%, 30% to 40%, 40% to 50%, 60% to 70%, 70% to 80%, 80% to 90%, 95% to 99% of the total population.

In yet other embodiments, following the identification and isolation of a monoclonal (bi-functional) surrogate antibody that has desirable ligand binding specificity, one of skill could further enhance the affinity of the molecule for the desired purpose by mutagenesizing the specificity region and screening for the tighter binding mutants. See, for example, Colas et al. (2000) Proc. Natl. Aca. Science 97:13720-13725.

The present invention will be better understood with reference to the following nonlimiting examples.

### Experimental

### EXAMPLE 1

### Process for Making a Ligand-Binding Surrogate Antibody Reagent Using a Non-Amplifiable Stabilization Strand

Surrogate Antibody (SAb) molecules were produced using self-assembling oligonucleotide strands (87nt+48nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind a BSA-Adipoyl-BZ101 conjugate and the unconjugated BZ101 (2,2′,4,5,5′ pentachlorobiphenyl) hapten.

Methods

A. Forming a Library of Surrogate Antibodies:

A library of 87 nt ssDNA oligonucleotides containing a random 40nt sequence, and FITC (F) and biotinylated (B) primers, were purchased from IDT. The 87nt ssDNA was designated #22-40-25 (87g2) to reflect the numbers of nucleotides in the constant sequence regions flanking the variable region. The is the specificity strand of the surrogate antibody molecule and the sequence of the 87mer is shown below (top strand; SEQ ID NO: 18), while the 48 nt oligonucleotide (stabilization strand) shown is below (bottom strand; SEQ ID NO: 19).

5′- GTA AAA CGA CGG CCA GTG TCT C - (40nt) - A GAT TCC TGT GTG AAA TTG TTA TCC -3′
||| ||| ||| ||| ||| || ||| ||| ||| ||| ||| |||
3′ - CAT TTT GCT GCC GGT CA ggagctctcg AGG ACA CAC TTT AAC AAT AGG- F5′

The two constant region nucleotide sequences on either side of the variable sequence are complementary to the nucleotide sequences of a juxtaposed 48nt. stabilization oligonucleotide. The stabilization strand is FITC-labeled 5′- and referenced as oligonucleotide (#F21-10-17) (bases in bold are non-complimentary to bases on the 87nt specificity strand):

Oligos were reconstituted in DI water to 0.1 mM (100 pm/μl) and stored as stock solutions in 2ml screw top vials at −20° C. (manufacturer claim for reconstituted stability is >6 months). Working aliquots of 20 μl each were dispensed into PCR reaction tubes and stored at −20° C.

B. Selection; Cycle 1

4 μl of 0.1 mM ssDNA oligonucleotide A22-40-25 (i.e. “+87”) library (2.4×10¹⁴ molecules) were mixed with 4 μl of 0.1 mM F21-10-17 (i.e. “−40”) that is FITC-labeled at 5′ end and 2 μl of 5×TNKMg5 (i.e. TNK buffer containing 5 mM MgSO4) buffer. TNK Buffer is a Tris Buffered Saline, pH 8.0. The 5× stock comprise 250 mM Tris HCl, 690 mM NaCl, 13.5 mM KCl and a working (1×) buffer comprises 50 mM Tris HCl, 138mM NaCl, and 2.7 mM KCl. TNK5 Mg is TNK above with 5 mM MgSO₄ (1:200 dilution of 1M MgSO₄ stock) and 5XTNK5Mg is 5XTNK with 25 mM MgSO4 (1:40 dilution of 1M MgSO₄).

Annealing of SAb molecules was performed using the HYBAID PCR EXPRESS thermal cycler. The oligo mixture was heated to 96° C. for 5′, the temperature was reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min. Total annealing time of 1.5 hours.

To assay for the formation of the surrogate antibody electrophoresis was employed. On each preparative gel, a FAM-87 and F-48 was loaded to demonstrate the location of the corresponding bands and SAb. On a parallel gel (or the other half of the preparative gel), a 10 bp ladder, 48ss, 87ss and the retentate PCR product next to an aliquot (0.5 μl) of each annealed SAb. 10 μl of reaction mixture from above was mixed with 7 μl, 60% w/v sucrose. Mixture was loaded onto a 20% acrylamide gel. The 48nt (F21-10-17) and dsSAb appeared as green fluorescent bands. The 48 band runs at approximately 50 base pairs and the dsSAb runs about 304. After extracting the Sab, the gel is stained with EtBr (1 μl of 10 mg/ml into 10 ml buffer). The 87 band will appear at approximately 157 bp, using the standard molecular weight function.

The gel fragment containing the SAB 87/48 band was excised and place in a 1.5 ml eppendorf tube. The gel fraction was macerated using a sterile pipette tip and 400 μl TNKMg5 buffer containing 0.05% v/v Tween 20 is added and the sample is then shaken on a rotating platform at the lowest speed for 2 hours/RT. The gel slurry was aspirated and added to a Pall Filter 300K and spun in Eppendorf 5417R at 1-5000×g (7000 rpm) for 3′. 40 μl TNKMg5 buffer containing 0.05% Tween was added to a volume ≦440 μl and centrifuge 3′.

The volume of filtrate is measured. RFU (relative fluorescence units) of the formed Sab was measured using a 10 μl aliquot of the filtrate and 90 μl buffer, and the Wallac VICTOR2, mdl 1420 (Program name “Fluorescein (485nm/535nm, 1”). A blank of buffer only was also measured. Total fluorescence was calculated by subtracting the background and multiplying by the appropriate dilution factor and volume.

1/10 volume (40 μl) MeOH was added to the filtrate along with 20 μl BSA-aa-BZ101 conjugate (1 μg/μl conjugate concentration in TNKMg5 Tw0.05 containing 10% MeOH v/v) to filtrate. The BSA-AA-BZ101 conjugate, synthesis, characterization was performed as outlined in Example 5. The sample was incubated for 1 hour/RT.

The reaction mixture was aspirated and added to a new Nanosep 100K Centrifugal Device and centrifuge at 1000g/3′. (The Nanosep 100K and 300K Centrifugal Devices were pruchaced form PALL-Gelman Cat #OD100C33 and are centrifugal filters with Omega low protein and DNA binding, modified polyethersulfone on polyethylene substrate.) The filters were used to fractionate SAb bound to BSA-AD-BZ101 from unbound Sab. SAb bound to the conjugate was recovered in the retentate while unbound SAb continued into the filtrate. The filtrate was aspirated and added to new 1.5 ml Eppendorf tube. 100 μl of mixture was removed and the RFU's was quantified in a microwell plate using Wallac Victor II. The retentate was washed only one time for cycle 1 (two times for cycle 2 and 3 times for cycles 3-6) at 1000g/3-8′ using 400 μl aliquots of TNKMg5 buffer (without Tween and MeOH). Spin times vary from filter to filter (generally 3-8 minutes). Retentate was saved for SAb, keep filtrate and pool to measure fluorescence×volume to coincide with retentate RFU. Filtrate was discarded.

SAb (when SAb is bound to conjugate, MW >1OOKD) in the retentate was recovered by adding a 100 μl aliquot of DI H₂O, swirling, and aspirating. The Total RFU's was calculated for the recovered material. Percent recovery was calculated by calculating total recovered vs. total in starting amount of SAb incubated with conjugate.

C. PCR Amplification

The DNA recovered from the retentate was amplified using a 40 cycle PCR amplification program and 2 μM of primer F22-5 and 2 uM of primer Bio21-4. Bio21-4 adds biotin to 5′ end of −87 oligonucleotide.

PCR Primers. The primers were designed to amplify only the 87 strand (the specificity strand) and not the −48 strand (the stabilization strand). This was accomplished by having 4-5 bases on the 3′ end that compliment the 87 strand but not the 48 strand. See FIG. 7. Four to five bases of non-complimentarity was sufficient to inhibit elongation.

The primer sequences used for PCR amplification were as follows. Primer F22-5—amplifies off of the −87 strand to make a new +87 and comprise the sequence: 5′ FAM—GTA AAA CGA CGG CCA GTG TCT C 3′(SEQ ID NO: 20). Primer Bio-21-4 -amplifies off of the +87 to make a biotin-labeled −87 that in some embodiments can be used to extract −87 strands that do not anneal to the −48. The sequence for Bio-21-4 is 5′ bio—GGA TAA CAA TTT CAC ACA GGA ATC T 3′ (SEQ ID NO: 21).

Primers were reconstituted in 10 mM Tris (EB) to 0.1 mM (100 pm/μl) and stored in 2ml screw top vial at −20° C. as a stock solution (claim for reconstituted stability is >6 months). Working aliquots of 20 μl were dispensed into PCR reaction tubes and stored frozen at −20° C.

PCR reaction: 10 μl of the retentate was added to a 0.2ml PCR tube. 5μl of Thermopol 10× buffer, 1 μl NTP stock solution (PCR dNTP, nucleotide triphosphates 10 mM (Invitrogen 18427.013) which contains a mixture of 10 mM of each of four nucleotides (A, G, C, T), 12 μL of 5M Betaine (Sigma B-0300) and 10 μl of 10 pmole/μl of each primer was added. QS to 49.5 μl with DI H₂O. The program was run with the following parameters: 3 min, 940-65°-720 30 sec each x 35, 10° hold. When PCR machine is at 96° 5 μl of Taq DNA Polymerase ((NEBiolabs cat# M0267S) 5 U/μL) is added the reaction is mixed and placed in PCR machine.

Following the PCR reaction, 5 μL of PCR product were run on a 3% Agarose 1000 gel or 4% E-gel with controls of 10 bp ladder and ss oligos to verify amplification and size of bands. The remaining amplified DNA is purified by salt precipitation using 100% ethanol. Specifically, 1/3 volume (100 μl) of 8M Ammonium Acetate is added to 200 μl of the amplified DNA. 2.6 times the combined (DNA+Ammonium Acetate) volume (˜780-800 ul) of cold absolute ethanol (˜20° C.) is added to the tube. The tube is swirled and stored on ice for 1 hr. The sample is centrifuged for 15′/14,000 g 4° C. in a refrigerated centrifuge. The supernatant liquid is removed without touching or destroying the pellet. 0.5 ml of 70% (V/V) ethanol is added. The sample is mixed gently and centrifuged for 5′/14,000g. The supernatant is removed without disturbing the pellet and evaporate to dryness by exposing to air at RT.

When amplifying selected DNA from retentate, the following controls are also run: no DNA, 87 alone, and 48 alone. This will assure that the bands from the retentate are the right size and are not due to primer dimers. It will also show that the 48 strand is not amplifying in the SAb tube. By itself, the −48 will amplify and can be detected in the −48 control tube. This will identify the position of the ds 48 in the SAb tube if it was amplified.

Reannealing: The pellet was reconstituted by adding 8 μl of a solution containing 4 μl of sterile DI H₂O+4 μl of 0.1 mM −48nt oligonucleotide (F21-10-17). The sample was transferred to a 0.2 ml PCR tube and 2 μl of 5×TNKMg5 buffer was added. (Note; the addition of excess F21-10-17 (-48nt) primer drives the formation of the desired +87/−48 SAb molecules).

D. Cycle 2-6: Annealing SAb

The dsSAb was annealed by heating the reconstituted material in a 0.2 ml PCR tube using the temperature program previously specified for annealing. After the first cycle, multiple bands appear. Thus a parallel SAb aliquot was run with its corresponding PCR starting strands to verify that the band being cut out is in fact the new SAb. To verify that the SAb band was ds 87/48, an aliquot was removed and run on a denaturing gel (16%, boiling in 2× urea sample buffer) to verify that the band from the preparative gel contains both 87 and 48 strands.

Electrophoresis was performed at 120v for 40 min. 7 μl of 60% w/v sucrose was mixed with 10 μl of DNA and the sample is loaded. Any DNA component with FITC at 5′ end (i.e. SAb 87/48, ds 48 and ss48) will appear on the gel as a green fluorescent band under long wavelength. Run 5 pMol of F21-10-17 (−48nt primer) in an available lane as a size marker. SAb will be observed to co-migrate with 250-300 nt dsDNA in 20% acrylamide native gel. The SAb-gel section was excised and macerated in 250 μl of TNKMg5 Tw0.05 buffer. The sample was incubated for 2 hrs/RT while agitating on rotating platform at the lowest speed.

The gel suspension was transferred to a Pall 300K Centrifugal Device and centrifuge at 1-5000 g/3′ to remove the polyacrylamide. The retentate was washed by adding a 50 μl aliquot of buffer, centrifuge at 1000 g/3′. The SAb is recovered from the filtrate for use in subsequent selection cycle.

The RFU's of SAb and buffer blank was measured as describe above using a 100 ul aliquot of the filtrate on the Wallac Victor2.

E. Selection Cycles 2-7

1/10 volume of MeOH was added and 20 μl BZ110-aa-BSA (1 μg/μl) as in cycle 1. The sample was incubated for 1 hr and selected using Pall 100K filter. RFU measurement of the retentate after 2 washes for cycle 2 and 3 washes for cycle 3-6 were taken. Subtraction of the background RFU allow the determination of the % recovery.

Negative Selection. In this example, negative selection using BSA was not performed in Cycle #1-6.

When negative selection was desired, 250 μL of SAb 87/48 filtrate (2-20 pMol by FITC) was mixed with 20 μl of a 1 μg/μl (20 μg) BSA solution. The sample is Incubated for 30′/RT. The RFU's was measured in 100 ul aliquot using Wallac VICTOR II Program.

250 ul of the above reaction mix (20 μl is saved for 16% non-denaturing PAGE and 8% denaturing PAGE with 8M urea) was added to Nanosep 100K Centrifugal concentrator. The filter was centrifuged at 1000 g/15′/RT. Total volume in filtrate was −240 μl. Aspirate filtrate and place in new 1.5 ml Eppendorf tube. RFU's of 100 μl aliquot were checked.

The filter was washed by adding 200 μl TNKMg5 buffer, centrifuge (1000 g/10′/RT), add additional 200 μl of same buffer after centrifugation, re-centrifuge, add 100 μl of same buffer and centrifuge again. 100 μl DI H₂O was added, filtered, swirled and aspirate retentate. RFU's were determined on Wallac VICTOR II of SAb bound to BSA by aspirating retentate and % recovery was determined. 200 μl of negatively selected filtrate was mixed with 20 μl (1 μg/μl) of the BSA-aa-BZ10 conjugate suspended in TNKMg5 buffer. The mixture was incubated for 1 hour/RT with a total volume of 220 μl. The reaction solution was added to a new Nanosep 100 K centrifugal device and centrifuged at 1000 g/3′. A wash was performed 3 times using a TNKMg5 buffer. Measure RFU's of a 100 μl aliquot of the filtrate to determine % of unbound (free) SAb.

100 μl of DI H₂O was added to filter, swirled, and the retentate was aspirated. The entire sample was placed in a microtiter plate well. RFU's of sample were measured and background and calculate % Recovery.

Additional Steps. 1-20% of the bound SAb recovered in the 100 μl aliquot was used for PCR amplification with primer. This will again generate dsDNA in 4 tubes each containing 50 μl, as described previously. Cycles of negative and positive selection were repeated until no further enrichment in % recovery was observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate is amplified. The process of Polyclonal SAb production proceeds through 1. Binding, 2. Specificity Enhancement, 3. Affinity Enhancement, prior to production of monoclonal SAb clones.

Calculations. The total amount of RFU's in the recovered conjugate-binding aliquot vs. the total amount of RFU's that were present when incubated with the conjugate was determined. For negative selection; the amount of RFU's in the recovered BSA-binding aliquot vs. the total amount of RFUs present when incubated with BSA was determined. RFUs quantified from filtrate provides supportive data and information indicating unbound SAb and loss on filter device.

Notes: The DNA/conjugate and DNA/BSA ratios in cycles #2-5 was 10-100 nM DNA/2,000 nM protein, or 1 molecule of SAb to 20-200 molecules of the conjugate or BSA. This calculation assumes that the conjugate has the reported 20 moles of BZ101 per mole of protein). The molecular weight of the (SAb 87/48--BSA-aa-BZ101) complex=(A22-40-25=27.4 Kd)+(FM21-10-17=15.4 Kd)+(BSA=67Kd)+(20 BZ101=7 Kd). Total=˜116.8 Kd; 2SAb: 1 Conjugate=159.6 Kd.

### EXAMPLE 2

### Monoclonal SAb Preparation

The polyclonal SAb population is amplified by PCR to produce double stranded 78nt and double stranded 40nt molecules using specific primers. Amplification artifacts and PCR-errors are minimized by using polymerase with high fidelity and low number PCR cycles 1(25 cycles). PCR products are electrophoresed in 3½ high resolution agarose gel and 78 nucleotide fragments are recovered and purified by Qiagen Gel extraction kid. The purified 78nt double strand DNA are cloned into PCR cloning vector (such as pGEM-T-Easy) to produce plasmid containing individual copies of the ds 78nt fragment. The E. coli bacteria (e.g. strain JM 109, Promega) are transformed with the plasmids by electroporation.

The transformed bacteria are cultured on LB/agar plates containing 100 μg/ml Ampicillin. Bacteria containing the 78nt fragment produce white colonies and bacteria that do not contain the 78nt fragment expresses 13gal and form blue colonies. Individual white colonies are transferred into liquid growth media in microwells (e.g. SOC media, Promega) and incubated overnight at 37° C.

The contents of the wells are amplified after transferring an aliquot from each well into a PCR microplate. The need to purify the PCR product is avoided by using appropriate primer and PCR conditions. SAb molecules are assembled in microplates using the previously cited process of adding 40nt-fragments and hybridization in a thermalcycler using a defined heating and cooling cycle.

### EXAMPLE 3

### Analysis and Database Construction

Reactive panel profiling of monoclonal SAb clones is used to compare binding characteristics used in selecting reagent(s) for commercial application. Characteristics that are analyzed can include:
1) recognition of target ligand;
2) relative titer and affinity;
3) sensitivity;
4) specificity;
5) matrix effects;
6) temperature effects;
7) stability; and
8) other variables of commercial significance (e.g., lysis, effector function).

Standard test protocols are used and data collected from each clone is entered into a relational database.

Characterization assays transfer aliquots of assembled monoclonal SAb reagents to specific characterization plates for analysis. Affinity and titration assays compare relative affinity (Ka) and concentration of each reagent. Sensitivity assays compare the ability to detect low concentrations of the target ligand and provide an estimate of Least Detectable Dose. Specificity assays compare SAb recognition of irrelevant/undesirable ligands. Matrix interference studies evaluate the effect of anticipated matrix constituents on the binding of SAb. Temperature effects evaluate the relationship to binding. Stability identifies the most stable clones and problems requiring further evaluation. Other characteristics relevant to the anticipated application can also be evaluated using known means.

### EXAMPLE 4

### Preparation of Surrogate Antibody 78/48 to PCB Congener BZ101

Surrogate Antibody (SAb) molecules were produced using self-assembling oligonucleotide strands (78nt+48nt) to form a dimeric surrogate antibody molecule having a 40 nt random sequence binding loop with adjacent constant nucleotide sequences. Cycles of ligand binding, PCR amplification, bound/free separation, and reassembly/reannealing were used to enrich the SAb population with molecules that would bind a BSA-Adipoyl-BZ11 conjugate and the unconjugated BZ101 (2,2′,4,5,5′ pentachlorobiphenyl) hapten.

A. Selection; Cycle 1

Forming the surrogate antibody: The library of surrogate antibodies used in the following experiment was formed as follows. A library of 78 nt ssDNA oligonucleotides containing a random 40nt sequence, and FITC (F) and biotinylated (B) primers, were purchased from Gibco-Invitrogen life technologies. The 78nt ssDNA was designated #17-40-21 to reflect the numbers of nucleotides in the constant sequence regions flanking the variable region. The sequence of the 78mer (i.e., the specificity strand; SEQ ID NO: 22) is shown below along with the 48 nt oligonucleotide (i.e., the stabilization strand; SEQ ID NO: 23).

(78nt oligonucleotide. shown as top strand)
5′ GTA AAA CGA CGG CCA GT - (40nt) - TCC TGT GTG AAA TTG TTA TCC 3′
||| ||| ||| ||| ||| || ||| ||| ||| ||| ||| |||
3′ CAT TTT GCT GCC GGT CA ggagctctcg AGG ACA CAC TTT AAC AAT AGGF5′
(48 nt oligonucleotide shown as bottom strand)

The two constant region nucleotide sequences on either side of the variable sequence are complementary to the nucleotide sequences of a juxtaposed 48nt stabilization oligonucleotide. The bases in bold of the FITC-labeled 5′-oligonucleotide (#F21-10-17) are non-complimentary to bases on the 78nt strand. Oligos were reconstituted in DI water to 0.1 mM (100 pm/μl) and stored as stock solutions in 2ml screw top vials at −20° C. 4 μl of 0.1 mM ssDNA oligonucleotide A17-40-21 (i.e. “+78”) library (2.4×10¹⁴ molecules) (i.e., specificity strand) was mixed with 4 μl of 0.1 mM F21-10-17 (i.e. “−40”) (stabilization strand) that is FITC-labeled at 5′ end and 2 μl of 5×TNKMg5 (i.e. TNK buffer containing 5mM MgSO4) buffer. TNK Buffer is Tris Buffered Saline, pH 8.0 (a 1× stock comprises 50 mM Tris HCl 138mM NaCl and 2.7 mM KCl). The TNKMg5 buffer comprises the TNK buffer plus 5 mM MgSO₄.

SAb molecules were annealed using the HYBAID PCR EXPRESS thermal cycler (program name: “Primer”). The oligo mixture is heated to 96° C. for 5′, the temperature is reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min.

10 μl of reaction mixture from above was mixed with 7 μl, 60% w/v sucrose and loaded onto a 1 mm 16% acrylamide gel (19:1 ratio Acrylamide:Methylene Bisacylamide). The gel was examined using long wave UV-366 mn BLAK-RAY LAMP model UVL-56. The 40nt (F21-10-17) and dsSAb appear as green fluorescent bands.

The “SAb 78/48” band was excised from the gel and the gel fraction was mascerated in 400 μl TNKMg5 buffer containing 0.05% v/v Tween 20. The gel slice was then shook on a vortex at the lowest speed for 2 hours/RT.

The gel slurry was aspirated and the gel suspension is added to an Amicon (Microcon) Centrifugal Device and spin at 1000 g/10′. 40 μl TNKMg5 buffer containing 0.05% Tween was added and the sample was centrifuge at 1000 g/10′. Total volume ≦440 μl.

40 μl MeOH was added to the filtrate. To quantify the amount of antibody, RFU (relative fluorescence units) was measured using a 100 μl aliquot of the filtrate and the Wallac VICTOR2, mdl 1420 (Program name “Fluorocein (485 nm/535 nm, 1″).

All of the SAb filtrate was added to the Nanosep 100K Centrifugal Device (Pall-Gelman) and it was Centrifuge at 1000 g/15′. RFU was quantified using a 100 μl aliquot of the filtrate as above.

B. Selection of Surrogate Antibody

The filtrate from above is added to a 0.2 ml PCR tube containing 20 μl BSA-aa-BZ101 conjugate (1 μg/μl conjugate concentration) in TNKMg5 Tw 0.05 containing 10% MeOH v/v). BSA-AA-BZ11 conjugate was synthesized as described below. Methanol added to 10%v/v final concentration. Tween 20 was added to 0.05% w/v final concentration. The sample was incubated for 1 hour/RT.

The reaction mixture was aspirated and added to new Nanosep 100K Centrifugal Device and centrifuge at 1000 g/10′. The Nanosep 100K Centrifugal Devices (Cat #OD100C33 PALL-Gelman, centrifugal filter with Omega low protein and DNA binding, modified polyethersulfone on polyethylene substrate) used was able to fractionate SAb bound to BSA-AD-BZ11 from unbound SAb. SAb bound to the conjugate was recovered in the retentate while unbound SAb continued into the filtrate. The filtrate was aspirated and added to new 1.5 ml Eppindorf tube. 100 μl was taken and the RFU's were quantified in a microwell plate using Wallac Victor II. The retentate was washed 3 times at 1000 g/10′ using 200 μl aliquots of TNKMg5 buffer (sans tween and MeOH). The filtrate was discarded.

SAb (when SAb is bound to conjugate, MW>100 KD) in the retentate was recovered by adding a 100 μl aliquot of DI H₂O, swirling, and apirating. The Total RFU's was calculated for the recovered material. % recovery was determined by calculating total recovered vs. total in starting amount of SAb incubated with conjugate.

C. PCR Amplification

The DNA recovered from the retentate was amplified using a 40 cycle PCR amplification program and 2 μM of primer FM13-20 and 2 uM of primer BioM13R48. BioM13R48 adds biotin to the 5′ end of +78 oligonucleotide. The PCR reaction amplifies +78nt, −48nt, −78nt and +48nt strands thereby reducing the theoretical yield of SAb

The primer sequences used for the PCR amplification are as follows: Primer #FM13-20 (SEQ ID NO: 24) has the sequence 5° FITC-GTA AAA CGA CGG CCA GT 3′ were FITC is fluorocein isothiocyanate and Primer #BioM13R48 (SEQ ID NO: 25) has the sequence 5′ Bio-GGA TAA CAA TTT CAC ACA GGA 3′ where Bio is biotin. The primers were reconstituted in DI water to 0.1 mM (100 pm/μl) and stored in 2 ml screw top vial at −20° C. as a stock solution.

100 μl of the retentate was added to a 0.2 ml PCR tube. 20 μl of Thermopol 10× buffer, 4 μl NTP stock solution, and 4 μl of 100 pmole/μl of each primer was added. The final volume was brought to 200 μl with DI H₂O. The samples were mixed and placed in PCR machine. When the temperature reaches 96° C. the program was pauses and 2 μl Deep Vent (exonuclease negative) DNA Polymerase stock solution (2 units/μl) (New England BioLabs cat #MO 259S) was added with 10× ThermoPol Reaction Buffer. 10× ThermoPol buffer comprises 10 mM KCL, 10 mM (NH4)₂SO₄, 20 mM Tris-HCL (pH8.8, 2° C.), 2 mM MgSO4, and 0.1% Triton X-100. The reaction mixture was aliquoted into empty 50 μl PCR tubes preheated in the machine to 96° C. The total amplification time was about 2.5-3 hours.

The amplified DNA was purified by extraction with an equal volume of a phenol-chloroform-isoamyl Alcohol solution (25:24:1 v/v). 200 μl of the amplified DNA was transferred to a 1.5 ml Eppindorf tube. 200 μl of the extraction solution was added to the tube. The tube was swirled and then centrifuged for 5′/12,000 g. The supernatant (buffer layer) was aspirated and transferred to a new 1.5 ml Eppindorf tube.

The aspirated DNA solution undergoes salt precipitation using 100% ethanol. 100 μl of 8M Ammonium Acetate was added to −200 μl of the aspirated DNA. 2.6 times the combined (DNA+Ammonium Acetate) volume (˜780-800 μl) of cold absolute ethanol (−20° C.) was added to the tube. The tube was mixed and store in ice water for 30′. The sample was centrifuged for 15′/12,000 g. The supernatant was aspirated and discarded. 0.5 ml of 70% (V/V) ethanol was added and the sample was centrifuged for 5′/12,000g. The supernatant was removed without disturbing the pellet and evaporate to dryness by exposing to air at RT. The pellet was reconstituted by adding 8 μl of a solution containing 4 μl of sterile DI H₂0+4 μl of 0.1 mM primer (F21-10-17). The sample is transferred to a 0.2ml PCR tube and 2 μl of 5× TNKMg5 buffer is added. The surrogate antibody was reformed by the addition of excess F21-10-17 (−48nt) primer favors the formation of the desired +78/−48 SAb molecules.

D. Annealing the SAb

The dsSAb was annealed by heating the reconstituted material in a 0.2ml PCR tube using the temperature program previously specified for annealing. 7 μl of 60% w/v sucrose with 10 μl of DNA and load sample onto a 16% acrylamide gel. Any DNA component with FITC at 5′ end (i.e. SAb 78/48, ds 48 and ss48) will appear on the gel as a green fluorescent band under long wavelength (UV-366 nm BLAK-RAY LAMP model UVL-56). The 5 pMol of F21-10-17 (-48nt primer) was also run on the gel as a size marker. The SAb 78/48 will be observed to co-migrate with 500-600nt dsDNA. The SAb-gel section was excised and mascerated and 250 μl of TNKMg5 Tw 0.05 buffer was added to the sample. The sample was then incubated for 2 hrs/RT while agitating on vortex at the lowest speed.

The gel suspension was transferred to an Amicon PCR Centrifugal Device and centrifuge at 1000 g/10′ to remove the polyacrylamide. The retentate was washed by adding a 50 μl aliquot of buffer, centrifuge at 1000 g/10′. The recovered SAb from the filtrate for use in subsequent selection cycle. The Sab was quantified by FU's using a 100 μl aliquot of the filtrate on the Wallac Victor2.

E. Selection Cycles 2-7

Negative selection using BSA was not performed in Cycle #1. The negative selection mixture comprises 250 μl of SAb 78/48 filtrate (2-20 pMol by FITC) with 20 μl of a 1 μg/μl (20 μg) BSA solution. The sample was incubate for 30′/RT and the RFU's of 100 μl aliquot using Wallac VICTOR II was measured. 250 μl of the above reaction mix (20 μl is saved for 16% non-denaturing PAGE and 8% denaturing PAGE with 8M urea) is added to Nanosep 100K Centrifugal concentrator. The filter was centrifuged at 1000 g/15′/RT. The total volume in filtrate was ˜240 μl.The filtrate is aspriated and place in a new 1.5 ml Eppindorf tube. The RFU's of a 100 μl aliquot was determined.

The filter was washed by adding 200 μl TNKMg5 buffer, centrifuge (1000 g/10′/RT), and an additional 200 μl of same buffer was added after centrifugation. The sample was re-centifuged and 100 μl of same buffer was added. The sample was centrifuged again. 100 μl DI H₂O was added to filter and swirled and the retentate is aspirated. The RFU's was determined on Wallac VICTOR II of SAb bound to BSA by aspirating retentate and determining % recovery.

200 μl of negatively selected filtrate was mixed with 20 μl (1 μg/μl) of the BSA-aa-BZ10 conjugate suspended in TNKMg5 buffer. The sample was ncubated for Ihour/RT. Total volume of the reaction is 220 μl.

The reaction solution was added to a new Nanosep 100K centrifugal device and centrifuged at 1000 g/15′. The filter was wash 3 time using TNKMg5 buffer. RFU's of a 100 μl aliquot of the filtrate was determined along with the % of unbound (free) SAb. 100 μl of DI H₂O was added to the filter, swirled, and the retentate aspirated. The entire sample was placed in a microtiter plate well and the RFU's and % recovery was measured.

From 1-20% of the bound SAb recovered in the 100 μl aliquot for PCR amplification was used with primer #BioM13R48 (100 pMol) and FM13-20 (100 pMol). This will again generate dsDNA in 4 tubes each containing 50 μl as described previously. Cycles of negative and positive selection are repeated until no further enrichment in % recovery is observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate was amplified. The process of Polyclonal SAb production proceeds through 1) binding, 2) specificity enhancement, and 3) affinity enhancement prior to production of monoclonal SAb clones.

F. Calculations

The total amount of RFU's in the recovered conjugate-binding aliquot vs. the total amount of RFU's that were present when incubated with the conjugate represents the % of the surrogate antibody bound.

For negative selection, the amount of RFU's in the recovered BSA-binding aliquot vs. the total amount of RFUs present when incubated with BSA is determined.

Additional calculations include RFUs quantified from the filtrate that provides supportive data and information indicating unbound SAb and loss on filter device.

Further note that the DNA/conjugate and DNA/BSA ratios in cycles #2-5 was 10-100 nM DNA/2,000 nM protein, or 1 molecule of SAb 78/48 to 20-200 molecules of the conjugate or BSA. This calculation assumes that the conjugate has the reported 20 moles of BZ101 per mole of protein. In addition, the molecular weight of the (SAb 78/48-BSA-aa-BZ101) complex is about 113.4Kd (A17-40-21=24 Kd)+(FM21-10-17=15.4 Kd)+(BSA=67 Kd)+(20 BZ101=7 Kd). The molecular weight of 2SAb:1 conjugate is ˜152.8Kd and the molecular weight of 1 SAb:2 conjugate ˜189.4 Kd.

Results

The production of surrogate antibody show in FIG. 1 was initiated to provide a more versatile core molecule than an aptamer having a stem-loop structure. The design incorporates constant region domains that bracket binding specificity domain. The multi-oligonucleotide structure allows for the simple attachment of multiple labels (e.g. FITC, biotin) that may, or may not be the same. Multiple, self-directed and self-forming, binding cavities can be readily incorporated. A stabilizing strand that is separate from the binding strand offers a convenient site for chemical modifications when required.

The surrogate antibodies are formed by annealing a “specificity-strand” to a “stabilizing-strand” prior to incubation with the target. Molecules that bind are amplified using asymmetric PCR that preferentially enriches the “specificity-strand”. The constant sequence “stabilizing-strand” is added, and surrogate molecules are annealed for another selection cycle.

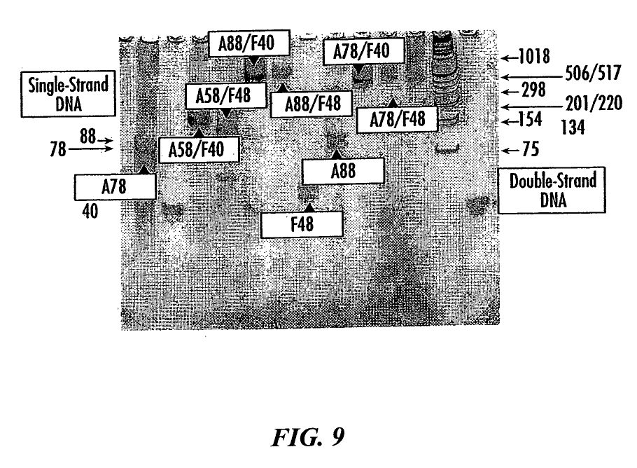

Surrogate antibodies can be assembled using “binding strands” that vary in the number of nucleotides in the binding loop. Each of these molecules will have a different binding cavity size and unique binding configurations. FIG. 8 illustrates the electrophoretic mobility of the surrogate antibodies that were assembled using different combinations of “specificity” and “stabilizing” primers. Fluorocein-labeled “stabilizing strands” (prefix “F”) and un-labeled “specificity strands” (prefix “A”) were used in the production of these molecules. This combination illustrates a significant shift in the electrophoretic mobility of the fluorocein-labeled “Stabilization” strand and the annealed molecule (FIG. 9). The lanes in FIG. 9 are as follows: Lane 1 primer A78, Lane 2 primer F40, Lane 3 Synthetide™ “A58/F40”, Lane 4 Synthetide™ “A58/F48” Lane 5 Synthetide™ “A88/F40”, Lane 6 Synthetide™ “A88/F48”, Lane 7 primer F48, Lane 8 primer A88, Lane 9 Synthetide™ “A78/F40”, Lane 10 Synthetide™ “A78/F48”, Lane 11 Synthetide™ “A78/F40, Lane 12 dsDNA markers (number of nucleotides in each strand indicated to right), Lane 13 primer F40.

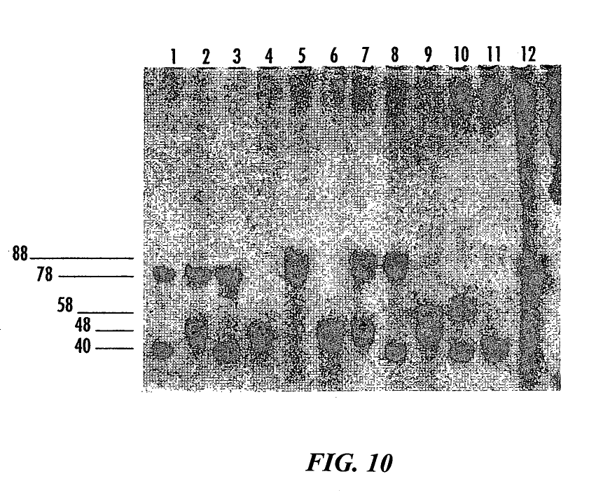

The surrogate antibodies that were characterized using non-denaturing acrylamide gel electrophoresis were re-characterized using a denaturing gel (8% acrylamide, 8M urea) to verify the duplex nature of the molecule and approximate 1:1 stoichiometry of the “specificity” and “stabilization” strands (FIG. 10). The lanes in FIG. 10 are as follows: Lane 1 A78/F40, Lane 2 A78/F48, Lane 3 A78/F40, Lane 4 Primer F48, L A88, Lane 6 F48, Lane 7 A88/F48, Lane 8 A88/F40, Lane 9 A58/48, Lane 10 Lane 11 F40, Lane 12 A78.

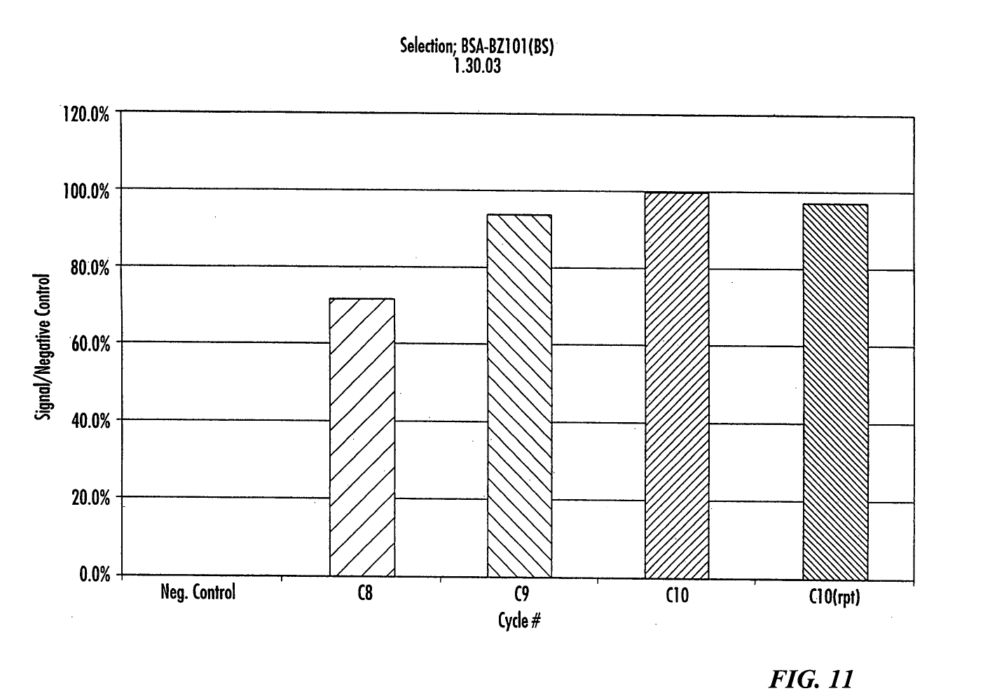

FIG. 11 illustrates the selection and enrichment of the surrogate antibodies to BSA-PCB (BZ101 congener) conjugates. Signal/Negative control represents as a percent the amount of surrogate antibody bound to the target verses the amount of surrogate antibody recovered when the target is absent (negative control).

### EXAMPLE 5

### Methods for Making a Ligand-Binding Surrogate Antibody Reagent that Recognizes IgG

As outlined in Example 1, surrogate antibody (SAb) molecules were produced using self-assembling oligonucleotide strands (87nt+48nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind an IgG polypeptide. Methods for the selection are discussed in detail in Example 1.

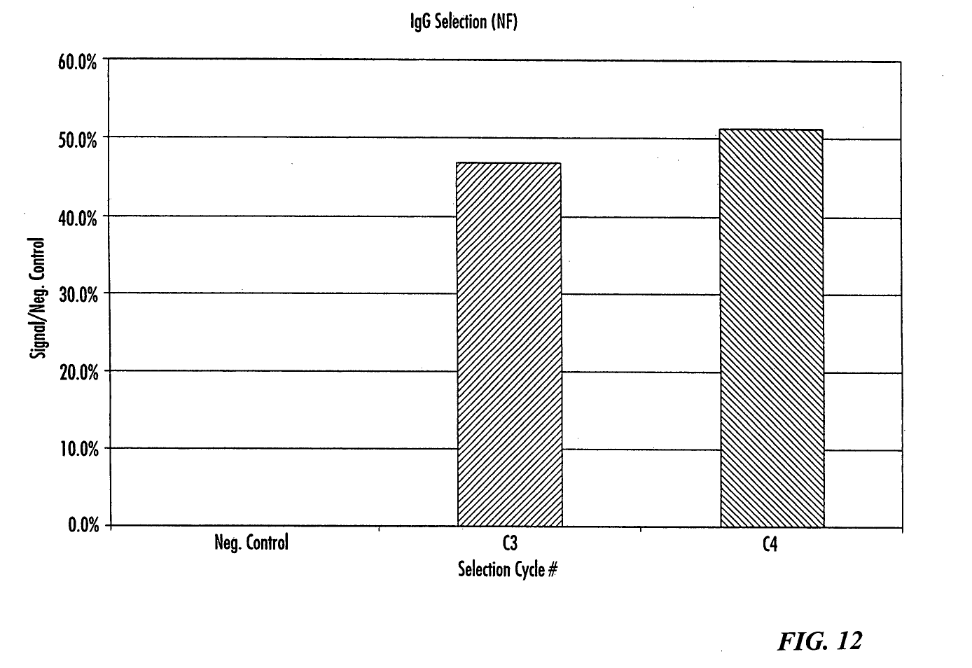

FIG. 12 illustrates the selection and enrichment of the surrogate antibodies to IgG. Signal/Negative control represents as a percent the amount of surrogate antibody bound to the target verses the amount of surrogate antibody recovered when the target is absent (negative control).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

1. An isolated bi-functional surrogate antibody molecule comprising a specificity strand and a stabilization strand,
said specificity strand comprising a nucleic acid sequence having a specificity region flanked by a first constant region and a second constant region;
said stabilization strand comprises a first stabilization domain that interacts with said first constant region and a second stabilization domain that interacts with said second constant region;
said bi-functional surrogate antibody further having attached thereto an immunomodulatory agent and,
said bi-functional surrogate antibody molecule is capable of interacting with a ligand of interest.

2. The isolated bi-functional surrogate antibody molecule of claim 1, wherein said stabilization strand and said specificity strand comprise distinct molecules.

3. The isolated bi-functional surrogate antibody molecule of claim 1, wherein said stabilization strand further comprises a first spacer domain between said first stabilization domain and said second stabilization domain.

4. The isolated bi-functional surrogate antibody molecule of claim 1, wherein said stabilization strand comprises an amino acid sequence.

5. The isolated bi-functional surrogate antibody molecule of claim 1, wherein said stabilization strand comprises a second nucleic acid sequence.

6. The isolated bi-functional surrogate antibody molecule of claim 5, wherein said immunomodulatory agent is attached to at least one of the stabilization strand, the first constant region, or the second constant region.

7. The isolated bi-functional surrogate antibody molecule of claim 5, wherein said immunomodulatory agent comprise an immunoglobulin constant region, an active fragment of the immunoglobulin constant region, or an active variant of the immunoglobulin constant region.

8. The isolated bi-functional surrogate antibody molecule of claim 7, wherein said immunomoglobulin constant region comprises an IgG immunoglobulin constant region, an active fragment of the IgG immunoglobulin constant region, or an active variant of the IgG immunoglobulin constant region.

9. The isolated bi-functional surrogate antibody molecule of claim 5, wherein said immunomodulatory agent comprises a cytokine, an active variant of the cytokine, an active fragment of the cytokine, a chemokine, an active variant of the chemokine, or an active fragment of the chemokine.

10. The isolated bi-functional surrogate antibody molecule of claim 5, wherein said immunomodulatory agent comprises a nucleic acid sequence comprising a CpG motif.

11. The isolated bi-functional surrogate antibody molecule of claim 10, wherein said CpG motif is immunostimulatory.

12. The isolated bi-functional surrogate antibody molecule of claim 5, wherein said immunomodulatory agent comprises a lipopolysaccharide or an active derivative of a lipopolysaccharide.

13. The isolated bi-functional surrogate antibody molecule of claim 5, wherein said immunomodulatory agent comprises a second specificity region, wherein said second specificity region is capable of interacting with an immune response regulator.

14. The isolated bi-functional surrogate antibody molecule of claim 13, wherein said immune response regulator comprises an FγR receptor.

15. The isolated bi-functional surrogate antibody molecule of claim 5, wherein said ligand of interest is selected from the group consisting of a polypeptide, a cell, a microbe, an organic molecule, or an inorganic molecule.

16. The isolated bi-functional surrogate antibody molecule of claim 15, wherein said microbe is a virus or a bacterium.

17. The isolated bi-functional surrogate antibody molecule of claim 15, wherein said cell is a cancer cell.

18. The isolated bi-functional surrogate antibody molecule of claim 5 further comprising a modified nucleotide having a modification at the 2′ position of a nucleotide sugar.

19. The isolated bi-functional surrogate antibody molecule of claim 5 further comprising a functional moiety that increases resistance to nuclease degradation.

20. The isolated molecule of claim 5 further comprising a functional moiety comprising a non-amplifiable moiety that increases resistance to polymerase activity in a PCR reaction.

21. A composition comprising the bi-functional surrogate antibody of claim 1.

22. A method of delivering an immunomodulatory agent to a ligand of interest comprising
a) administering to a subject a composition comprising an isolated bi-functional surrogate antibody molecule comprising a specificity strand and a stabilization strand,
said specificity strand comprising a nucleic acid sequence having a specificity region flanked by a first constant region and a second constant region;
said stabilization strand comprises a first stabilization domain that interacts with said first constant region and a second stabilization domain that interacts with said second constant region;
said immunomodulatory agent is attached to said bi-functional surrogate antibody molecule; and,
said bi-functional surrogate antibody molecule is capable of interacting with said ligand of interest.

23. The method of claim 22, wherein said stabilization strand and said specificity strand comprise distinct molecules.

24. The method of claim 22, wherein said stabilization strand comprises a second nucleic acid sequence.

25. The method of claim 22, wherein said immunomodulatory agent is attached to the stabilization strand, the first constant region, or the second constant region.

26. The method of claim 24, wherein said immunomodulatory agent comprises an immunoglobulin constant region, an active fragment of the immunoglobulin constant region, or an active variant of the immunoglobulin constant region.

27. The method of claim 26, wherein said immunoglobulin constant region comprises an IgG immunoglobulin constant region, an active fragment of the IgG immunoglobulin constant region, or an active variant of the IgG immunoglobulin constant region.

28. The method of claim 24, wherein said immunomodulatory agent comprises a cytokine, a active variant of the cytokine, an active fragment of the cytokine, a chemokine, an active variant of the chemokine, or an active fragment of the chemokine.

29. The method of claim 24, wherein said immunomodulatory agent comprises a nucleic acid sequence comprising a CpG motif.

30. The method of claim 29, wherein said CpG motif is immunostimulatory.

31. The method of claim 24, wherein said immunomodulatory agent comprises a lipopolysaccharide or an active derivative of the lipopolysaccharide.

32. The method of claim 24, wherein said immunomodulatory agent comprises a second specificity region capable of interacting with an immune response regulator.

33. The method of claim 32, wherein said immune response regulator comprises an FγR receptor.

34. The method of claim 24, wherein said ligand of interest is selected from the group consisting of a polypeptide, a cell, and a microbe.

35. The method of claim 34, wherein said microbe is a virus or a bacterium.

36. The method of claim 34, wherein said cell is a cancer cell.

37. A method for modulating an immune response against a ligand of interest in a mammalian subject comprising
administering to the mammalian subject an isolated bi-functional surrogate antibody molecule comprising a specificity strand and a stabilization strand,
said specificity strand comprising a nucleic acid sequence having a specificity region flanked by a first constant region and a second constant region;
said stabilization strand comprises a first stabilization domain that interacts with said first constant region and a second stabilization domain that interacts with said second constant region; and,
said bi-functional surrogate antibody having attached thereto an immunomodulatory agent; and,
said bi-functional surrogate antibody molecule is capable of interacting with said ligand of interest.

38. The method of claim 37, wherein said immune response is stimulated.


SEQUENCE LISTING (25 sequences)

SEQ ID NO: 1 - DNA, 87 bp, Artificial Sequence
  Oligonucleotide comprising “F48” stabilization strand of a synthetic antibody.
gtaaaacgacggccagtgtctcnnnnnnnnnnnnnnnnnnnnnnnnnnnnnnnnnnnnnnnnagattcctgtgtgaaattgttatcc

SEQ ID NO: 2 - DNA, 48 bp, Artificial Sequence
  Oligonucleotide comprising the “F22-40-25” specificity strand of a synthetic antibody.
ggataacaatttcacacaggagctctcgaggactggccgtcgttttac

SEQ ID NO: 3 - DNA, 24 bp, Artificial Sequence
  Primer B21-40
ggataacaatttcacacaggaatc

SEQ ID NO: 4 - DNA, 22 bp, Artificial Sequence
  Primer F17-50
gtaaaacgacggccagtgtctc

SEQ ID NO: 5 - DNA, 4 bp, Artificial Sequence
  Immunomodulatory nucleic acid motif.
ncgn

SEQ ID NO: 6 - DNA, 6 bp, Artificial Sequence
  Immunomodulatory nucleic acid motif.
gacgtt

SEQ ID NO: 7 - DNA, 6 bp, Artificial Sequence
  Immunomodulatory nucleic acid motif.
agcgtt

SEQ ID NO: 8 - DNA, 6 bp, Artificial Sequence
  Immunomodulatory nucleic acid motif.
aacgct

SEQ ID NO: 9 - DNA, 6 bp, Artificial Sequence
  Immunomodulatory nucleic acid motif.
gtcgtt

SEQ ID NO: 10 - DNA, 6 bp, Artificial Sequence
  Immunomodulatory nucleic acid motif.
aacgat

SEQ ID NO: 11 - DNA, 8 bp, Artificial Sequence
  Immunomodulatory nucleic acid motif.
tcaacgtt

SEQ ID NO: 12 - DNA, 6 bp, Artificial Sequence
  Immunomodulatory nucleic acid motif.
gtcgyt

SEQ ID NO: 13 - DNA, 7 bp, Artificial Sequence
  Immunomodulatory nucleic acid motif.
tgacgtt

SEQ ID NO: 14 - DNA, 7 bp, Artificial Sequence
  Immunomodulatory nucleic acid motif.
tgtcgyt

SEQ ID NO: 15 - DNA, 20 bp, Artificial Sequence
  Immunomodulatory nucleic acid motif.
tccatgtcgttcctgtcgtt

SEQ ID NO: 16 - DNA, 19 bp, Artificial Sequence
  Immunomodulatory nucleic acid motif.
tcctgacgttcctgacgtt

SEQ ID NO: 17 - DNA, 24 bp, Artificial Sequence
  Immunomodulatory nucleic acid motif.
tcgtcgttttgtcgttttgtcgtt

SEQ ID NO: 18 - DNA, 87 bp, Artificial Sequence; identical to SEQ ID NO: 1
  Oligonucleotide comprising a specificity strand of a synthetic antibody.

SEQ ID NO: 19 - DNA, 48 bp, Artificial Sequence; identical to SEQ ID NO: 2
  Oligonucleotide comprising a stabilization strand of a synthetic antibody.

SEQ ID NO: 20 - DNA, 22 bp, Artificial Sequence
  Primer F22-5
gtaaaacgacggccagtgtctc

SEQ ID NO: 21 - DNA, 25 bp, Artificial Sequence
  Primer Bio-21-4
ggataacaatttcacacaggaatct

SEQ ID NO: 22 - DNA, 78 bp, Artificial Sequence
  Oligonucleotide comprising the specificity strand of a synthetic antibody.
gtaaaacgacggccagtnnnnnnnnnnnnnnnnnnnnnnnnnnnnnnnnnnnnnnnntcctgtgtgaaattgttatcc

SEQ ID NO: 23 - DNA, 48 bp, Artificial Sequence
  Oligonucleotide comprising the stabilization strand of a synthetic antibody.
cattttgctgccggtcaggagctctcgaggacacactttaacaatagg

SEQ ID NO: 24 - DNA, 17 bp, Artificial Sequence
  Primer FM13-20
gtaaaacgacggccagt

SEQ ID NO: 25 - DNA, 21 bp, Artificial Sequence
  Primer BioM13R48
ggataacaatttcacacagga